(19) United States
(12) Patent Application Publication
(10) Pub. No.: US 2014/0154762 A1
(43) Pub. Date: Jun. 5, 2014

(54) Genetically Enhanced Cyanobacteria Lacking Functional Genes Conferring Biocide Resistance for the Production of Chemical Compounds

(71) Applicants: Ulf DUEHRING, Fredersdorf (DE); James LEE, Cockeysville, MD (US); Kerstin BAIER, Kleinmachnow (DE)

(72) Inventors: Ulf DUEHRING, Fredersdorf (DE); James LEE, Cockeysville, MD (US); Kerstin BAIER, Kleinmachnow (DE)

(73) Assignee: ALGENOL BIOFUELS INC., Fort Myers, FL (US)

(21) Appl. No.: 14/132,012

(22) Filed: Dec. 18, 2013

Related U.S. Application Data
(63) Continuation of application No. PCT/EP2012/062272, filed on Jun. 25, 2012.
(60) Provisional application No. 61/571,295, filed on Jun. 24, 2011.

(51) Int. Cl. C12P 7/06; C12N 15/82

(52) U.S. Cl. CPC C12P 7/065; C12N 15/8243
USPC 435/161; 435/257.2; 435/471

(57) ABSTRACT

One embodiment of the invention provides a genetically enhanced cyanobacterium producing a first chemical compound comprising:
a genetically enhanced genome with a first gene inactivation in a first essential or conditionally essential gene of the cyanobacterium, and
a first extrachromosomal plasmid harboring the first essential or conditionally essential gene and at least one first production gene for production of the first chemical compound,
wherein the cyanobacterium lacks a functional gene conferring biocide resistance.

These cyanobacteria can produce a first valuable chemical compound in long-term cultures without the need to employ genes conferring biocide resistance.

### CROSS-REFERENCE TO RELATED APPLICATIONS

This application is a continuation of International Application PCT/EP2012/062272, filed on Jun. 25, 2012, which claims priority to U.S. Provisional Application No. 61/571,295, filed Jun. 24, 2011.

### STATEMENT REGARDING FEDERALLY SPONSORED RESEARCH

Not Applicable.

### REFERENCE TO SEQUENCE LISTING

This application contains a sequence listing satisfying the requirements of 37 C.F.R. §§1.821-1.825. The Sequence Listing is named “ABR_SEQ_LIST_P011_{—}05_US_{—}12_{—}17_{—}13_ST25” and is 315 KB in size.

### FIELD OF THE INVENTION

This invention is related to the field of production of valuable chemical compounds by using genetically enhanced cyanobacterial cells.

### DEFINITIONS AND GENERAL EXPLANATIONS

The term “biocide” refers to a chemical substance which is able to inhibit the growth of the cyanobacterial cells or even kill cyanobacterial cells, which are not resistant to this biocide. The term “biocide” can include herbicides, algaecides and antibiotics, which can inhibit the growth of the cyanobacteria. Non-limiting examples of the most commonly used antibiotics are kanamycin, ampicillin, neomycin and erythromycin.

Database entry numbers given in the following are for the CyanoBase, the genome database for cyanobacteria (http://bacteria.kazusa.or.jp/cyanobase/index.html); Yazukazu et al. “CyanoBase, the genome database for Synechocystis sp. Strain PCC6803: status for the year 2000”, Nucleic Acid Research, 2000, Vol. 18, page 72.

The EC numbers cited throughout this patent application are enzyme commission numbers which is a numerical classification scheme for enzymes based on the chemical reactions which are catalyzed by the enzymes.

As used herein, the term “genetically enhanced” refers to any change in the endogenous genome of a wild type cyanobacterial cell or to the addition of endogenous and non-endogenous, exogenous genetic code to a wild type cyanobacterial cell, for example the introduction of a heterologous gene. More specifically, such changes are made by the hand of man through the use of recombinant DNA technology or mutagenesis. The changes can involve protein coding sequences or non-protein coding sequences in the genome such regulatory sequences as non-coding RNA, antisense RNA, promoters or enhancers. Aspects of the invention utilize techniques and methods common to the fields of molecular biology, microbiology and cell culture. Useful laboratory references for these types of methodologies are readily available to those skilled in the art. See, for example, Molecular Cloning: A Laboratory Manual (Third Edition), Sambrook, J., et al. (2001) Cold Spring Harbor Laboratory Press; Current Protocols in Microbiology (2007) Edited by Coico, R, et al., John Wiley and Sons, Inc.; The Molecular Biology of Cyanobacteria (1994) Donald Bryant (Ed.), Springer Netherlands; Handbook Of Microalgal Culture: Biotechnology And Applied Phycology (2003) Richmond, A.; (ed.), Blackwell Publishing; and “The cyanobacteria, molecular Biology, Genomics and Evolution”, Edited by Antonia Herrero and Enrique Flores, Caister Academic Press, Norfolk, UK, 2008.

It is well known to a person of ordinary skill in the art that large plasmids can be produced using techniques such as the ones described in the US patents U.S. Pat. No. 6,472,184 B1 titled “method for producing nucleic acid polymers” and U.S. Pat. No. 5,750,380 titled “DNA polymerase mediated synthesis of double stranded nucleic acid molecules”, which are hereby incorporated in their entirety.

Denominations of genes are in the following presented in a three letter lower case name followed by a capitalized letter if more than one related gene exists, for example pyrF or leuB. The respective protein encoded by that gene is denominated by the same name with the first letter capitalized, such as PyrF or LeuB.

Denominations for promoter sequences, which control the transcription of a certain gene in their natural environment are given by a capitalized letter “P” followed by the gene name according to the above described nomenclature, for example “PpetJ” for the promoter controlling the transcription of the petJ gene.

Denominations for enzyme names can be given in a two or three letter code indicating the origin of the enzyme, followed by the above mentioned three letter code for the enzyme itself, such as SynAdh (Zn²⁺ dependent Alcohol dehydrogenase from Synechocystis PCC6803), ZmPdc (pyruvate decarboxylase from Zymomonas mobilis)

The term “nucleic acid” is intended to include nucleic acid molecules, such as polynucleotides which include an open reading frame encoding a polypeptide, and can further include non-coding regulatory sequences of genes, such as promoters and enhancers as well as non-coding RNAs. In addition, the terms are intended to include one or more genes that are part of a functional operon. In addition the terms are intended to include a specific gene for a selected purpose. The gene can be endogenous to the host cell or can be recombinantly introduced into the host cell.

In a further aspect, the invention also provides nucleic acids, which are at least 60%, 70%, 80%, 90% or 95% identical to the promoter nucleic acids or to the nucleic acids encoding either the proteins for the first essential or conditionally essential genes or the first production genes for the production of the first chemical compound disclosed therein. With regard to the promoters, truncated versions of the promoters including only a small portion of the native promoters upstream of the transcription start point, such as the region ranging from −35 to the transcription start can often be used. The invention also provides amino acid sequences for enzymes for the production of first chemical compounds, which are at least 60%, 70%, 80%, 90% or 95% identical to the amino acid sequences disclosed therein.

The percentage of identity of two nucleic acid sequences or two amino acid sequences can be determined using the algorithm of Thompson et al. (Clustal W, 1994 Nucleic Acid Research 22: pages 4,673 to 4,680). A nucleotide sequence or an amino acid sequence can also be used as a so-called “query sequence” to perform a nucleic acid or amino acid sequence search against public nucleic acid or protein sequence databases in order to, for example identify further unknown homologous promoters, or homologous protein sequences and nucleic acid sequences which can also be used in embodiments of this invention. In addition, any nucleic acid sequences or protein sequences disclosed in this patent application can also be used as a “query sequence” in order to identify yet unknown sequences in public databases, which can encode for example new enzymes which could be useful in this invention. Such searches can be performed using the algorithm of Karlin and Altschul (1999 Proceedings of the National Academy of Sciences USA 87: pages 2264 to 2268), modified as in Karlin and Altschul (1993 Proceedings of the National Academy of Sciences USA, 90: pages 5873 to 5877). Such an algorithm is incorporated in the Nblast and Xblast programs of Altschul et al. (1999 Journal of Molecular Biology 215, pages 403 to 410) Suitable parameters for these database searches with these programs are, for example, a score of 100 and a word length of 12 for blast nucleotide searches as performed with the Nblast program. Blast protein searches are performed with the Xblast program with a score of 50 and a word length of 3. Where gaps exist between two sequences, gapped blast is utilized as described in Altschul et al. (1997 Nucleic Acid Research, 25: pages 3389 to 3402).

The term “genome” refers to the genome of the cyanobacterium without the first gene inactivation and excluding the recombinant first extrachromosomal plasmid, which is introduced into the genetically enhanced cyanobacterium via recombinant DNA technology. The term “genome” therefore refers to the chromosomal genome as well as to extrachromosomal plasmids which are normally present in the wild type cyanobacterium. For example, cyanobacteria such as Synechococcus PCC7002 can include up to 6 extrachromosomal plasmids in their wild type form.

The term “first essential gene” refers to a gene which under all circumstances and growth conditions is essential for the growth and also for the culturing of the genetically enhanced cyanobacterium. In contrast to that, the term “conditionally essential gene” refers to a gene which is only essential for the growth and culturing of the genetically enhanced cyanobacterium under certain growth conditions, but not under other conditions which are different. Examples for essential genes are the pryF gene encoding the orotidine-5′-monophosphate decarboxylase, an essential enzyme for the uracil biosynthesis pathway. Another example for an essential gene is the leuB gene, encoding the 3-isopropylmalate dehydrogenase, which is an essential enzyme for the leucine biosynthesis pathway. A gene inactivation in both of these essential genes therefore leads to an auxotrophy of the genetically enhanced cyanobacterium for either uracil or leucine. Additionally, the hisB gene, which encodes the imidazol glycerol-phosphate dehydratase, an enzyme of the histidine biosynthesis pathway, is also an essential gene, whose gene inactivation results in a histidine auxotrophy of the genetically enhanced cyanobacteria.

An example for a conditionally essential gene is the narB gene, which encodes the nitrate reductase conferring the ability to use nitrate as a sole nitrogen source. A first gene inactivation in the narB gene therefore results in the loss of ability to use nitrate as a sole nitrogen source. Supplementation of the cyanobacterial growth medium (BG11 medium) with ammonia or nitrite nevertheless allows growth of cyanobacteria harboring a first gene inactivation in the narB gene. Therefore, the narB gene is only essential in a growth medium which lacks both ammonia and nitrite.

Another example of a conditionally essential gene would be a first gene inactivation in the gene petJ which encodes the iron-containing electron carrier cytochrome C553 (also called cytochrom 6). A first gene inactivation in the petJ gene is not lethal to the genetically enhanced cyanobacteria under standard culturing conditions, if large amounts of copper are present in the growth medium. However, if a copper-free growth medium is used no functional petE gene product is produced which is a plastocyanine, an analogous copper-containing electron carrier. Thus the presence of copper is essential for the survival of a genetically enhanced cyanobacterium harboring a first gene inactivation in the petJ gene. Additional examples of conditionally essential genes, which can be inactivated via the first gene inactivation, are genes conferring resistance to metal or semi-metal ions, such as nrsRS (sll0797/sll0798) controlling the Ni²⁻-dependent induction of the nrsBACD operon, involved in Ni²⁻ sensing, ziaA (slr0798) and corT (sll0794/slr0797) encoding a cobalt-dependent transcriptional regulator/cobalt-transporting P-type ATPase from Synechocystis sp. PCC 6803. Inactivation of the genes nrsA (slr0794) or corT (slr0797 result in a Ni²⁻ or Co²⁻ sensitive phenotype [García-Domínguez M, Lopez-Maury L, Florencio F J, Reyes J C. A gene cluster involved in metal homeostasis in the cyanobacterium Synechocystis sp. strain PCC 6803. J Bacteriol. 2000 March; 182(6):1507-14.]. A first gene inactivation in ziaA confers sensitivity to Zn²⁺. A first gene inactivation in arsBHC (slr0944/slr0945/slr0946) encoding an arsenate-efflux transporter, arsenical resistance protein ArsH, arsenate reductase confers sensitivity to arsenate.

Further examples for conditionally essential genes are genes such as smtA from Synechococcus 7942, ahpC (alr4404) and pcs (alr0975) from Anabaena 7120. SmtA codes for a metallothionein from Synechococcus 7942 conferring resistance against Zn²⁺ and Cd²⁺. AhpC encodes a hydroperoxide reductase, conferring resistance against various peroxide species and the gene pcs also increases the stress tolerance of the cyanobacteria [Mishra Y, Chaurasia N, Rai L C. AhpC (alkyl hydroperoxide reductase) from Anabaena sp. PCC 7120 protects Escherichia coli from multiple abiotic stresses. Biochem Biophys Res Commun. 2009 Apr. 17; 381(4):606-11. Epub 2009 Feb. 25] and [Chaurasia N, Mishra Y, Rai L C. Cloning expression and analysis of phytochelatin synthase (pcs) gene from Anabaena sp. PCC 7120 offering multiple stress tolerance in E. coli. Biochem Biophys Res Commun. 2008 Nov. 7; 376(1):225-30. Epub 2008 Sep. 4.].

### BACKGROUND OF THE INVENTION

Various valuable products, such as biofuels like fatty acid esters or alcohols, functional foods, vitamins, pharma-ceuticals such as lactams, peptides and polyketides or terpenes and terpenoids and also biopolymers such as polyhydroxyalkanoates can be produced via genetically enhanced cyanobacteria. Usually, the cyanobacteria can be genetically enhanced by introducing exogenous nucleic acids into the cyanobacteria, which harbor genes for the production of the valuable compounds as well as a biocide conferring resistance genes such as antibiotic resistance genes (ABR genes). These ABR genes are required in order to provide a positive selection pressure for the genetically enhanced cyanobacteria in comparison to the wild type cyanobacteria or other contaminants. The antibiotic resistant cyanobacteria have to be cultivated in growth media containing the antibiotic, in order to ensure that the exogenous nucleic acid also harboring the gene for production of the valuable compounds is maintained during cultivation. Antibiotic resistance genes often confer resistance to widely used antibiotics such as tetracycline, neomycin, ampicillin and kanamycin. The presence of these antibiotic resistance genes in bacteria and cyanobacteria poses a couple of serious regulatory and health issues. Large-scale outdoor cultures of the genetically enhanced cyanobacteria should not contain any antibiotic resistance conferring genes. Furthermore, the antibiotic resistance genes also could be transferred from these cultures to pathogenic organisms thereby causing antibiotic resistant infections of mammals. Therefore, there is a need to develop novel genetically enhanced cyanobacterial strains which can overcome the above-mentioned disadvantages.

This task is solved by providing a genetically enhanced cyanobacterium according to the base claim 1. Further claims are directed to advantageous embodiments of the genetically enhanced cyanobacteria, to a method of producing valuable chemical compounds by culturing the genetically enhanced cyanobacteria and also to a method for producing the genetically enhanced cyanobacteria.

### SUMMARY OF INVENTION

The invention provides a genetically enhanced cyanobacterium producing a first chemical compound comprising:
a genetically enhanced genome with a first gene inactivation in a first essential or conditionally essential gene of the cyanobacterium, and
a first extrachromosomal plasmid harboring the first essential or conditionally essential gene and at least one first production gene for production of the first chemical compound,
wherein the cyanobacterium lacks a functional gene conferring biocide resistance.

Accordingly, in an aspect of the invention, a genetically enhanced cyanobacterium producing ethanol is provided, having a genetically enhanced genome with a first gene inactivation in a first essential or conditionally essential gene of the cyanobacterium selected from the group consisting of smtAB, leuB, ziaRA, corRT, narB and pyrF; and a first extrachromosomal plasmid harboring the first essential or conditionally essential gene and at least one first production gene for the production of ethanol, wherein the at least one first production gene for the production of ethanol encodes an enzyme selected from the group consisting of Adh, Pdc, AdhE and combinations thereof; where the cyanobacterium lacks a functional gene conferring biocide resistance, the genome harbors more than one copy of the first essential or conditionally essential gene, all copies of the first essential gene carry at least one gene inactivation, and the genetically enhanced cyanobacterium produces ethanol.

In another aspect of the invention, a method for producing ethanol is provided, by: a) obtaining a genetically enhanced cyanobacterium producing ethanol which has i) a genetically enhanced genome with a first gene inactivation in a first essential or conditionally essential gene of the cyanobacterium selected from the group consisting of smtAB, leuB, ziaRA, corRT, narB and pyrF; and ii) a first extrachromosomal plasmid harboring the first essential or conditionally essential gene and at least one first production gene for the production of ethanol, where the at least one first production gene for the production of ethanol encodes an enzyme selected from the group consisting of Adh, Pdc, AdhE and combinations thereof; the cyanobacterium lacks a functional gene conferring biocide resistance, the genome harbors more than one copy of the first essential or conditionally essential gene with all copies of the first essential gene carry at least one gene inactivation; and b) culturing said genetically enhanced cyanobacterium in the absence of a biocide, the cyanobacterium producing ethanol; and c) recovering the ethanol.

### DESCRIPTION OF THE INVENTION

One embodiment of the invention provides a genetically enhanced cyanobacterium producing a first chemical compound comprising:
a genetically enhanced genome with a first gene inactivation in a first essential or conditionally essential gene of the cyanobacterium, and
a first extrachromosomal plasmid harboring the first essential or conditionally essential gene and at least one first production gene for production of the first chemical compound,
wherein the cyanobacterium lacks a functional gene conferring biocide resistance.

The genetically enhanced cyanobacterium is auxotrophic for a first essential factor, which is either produced involving a first essential biocatalyst encoded by the first essential gene or the first essential gene itself encodes this first essential factor. The first extrachromosomal plasmid, which harbors the first essential or conditionally essential gene and the at least one first production gene complements for the first gene inactivation in the genome of the genetically enhanced cyanobacterium. The first extrachromosomal plasmid is therefore required by the genetically enhanced cyanobacterium in order to restore the prototrophy for the first essential factor so that the first extrachromosomal plasmid is stably maintained within the genetically enhanced cyanobacteria even under long-term culturing conditions. There is no need for the genetically enhanced cyanobacterium to further contain a functional gene conferring biocide resistance, owing to this complementation strategy.

The first extrachromosomal plasmid can either harbor the exact same first essential or conditionally essential gene, which was inactivated by the first gene inactivation, or more preferred especially in the case that the inactivation of the first essential gene is realized by partial deletion can also contain homologous genes, having a high degree of sequence identity to the first essential or conditionally essential genes, which were inactivated via the first gene inactivation. If the first extrachromosomal plasmid includes a first essential gene, which is homologous to the first essential gene inactivated in the genome of the cyanobacterium, the possibility of a reconstitution of the first essential gene in the genome of the cyanobacterium via homologous recombination can be reduced. The lower the homology is, the lower the chance of homologous recombination. It is also possible to include the same first essential gene inactivated in the genome of the cyanobacterium in the extrachromosomal plasmid if the complete first essential gene was deleted, since a complete deletion of the first essential gene in the genome abolishes the possibility of a homologous recombination. Typical examples for the substitution of first essential genes by homologous essential genes are the replacement of leuB6803 (slr1517 from Synechocystis sp. PCC6803) with leuB7120 (alr1313 from Anabaena sp. PCC7120) having a sequence identity of 79.9% in comparison to the nucleic acid sequence of slr1517. Another example would be the replacement of pyrF6803 (sll0838 from Synechocystis sp. PCC6803) with pyrF7120 (alr2983 from Anabaena sp. PCC7120) having a sequence identity of 58.5% in comparison to the nucleotide sequence of sll0838. Furthermore the first essential or conditionally essential gene can also be an analogous gene encoding an analogous protein, which harbors a similar enzymatic function as the first biocatalyst encoded by the first essential or conditionally essential gene, but which only shares a low amino acid sequence identity, such as 50% to the first biocatalyst. In general, analogous proteins share related protein folds, but have unrelated sequences and developed independently the same protein fold during evolution. In particular, analogous first essential or conditionally essential genes on the first extrachromosomal plasmid encode for analogous proteins in the sense of the present invention if these genes are able to promote a complete genetic segregation with regard to the first gene inactivation in the cyanobacterium. Proteins, which are analogous to a first essential or conditionally essential biocatalyst, can be identified via a protein BLAST search via the National Center For Biotechnology Information (NCBI) by using the search parameters Word size: 3, Expect value: 10, Hitlist size: 100, Gapcosts: 11, 1, Matrix: BLOSUM62, Filter string: F, Genetic Code: 1, Window Size: 40, Threshold: 11, Composition-based stats: 2. Using this program, an analogous protein, a heavy metal translocating P-type ATPase from Bacillus cereus AH1273 sharing only 55% sequence identity with ziaA from Synechocystis sp. PCC 6803 (slr0798) can be identified.

In a further variant of the genetically enhanced cyanobacteria of the invention, the first valuable chemical compound is selected from various alcohols, such as ethanol, propanol or butanol, alkanes and alkenes, resp. such as ethylene or propylene, biopolymers such as polyhdyroxy-alkanoates like polyhydroxybutyrate, fatty acids, fatty acid esters, carboxylic acids such a amino acids, terpenes and terpenoids. Furthermore, the first valuable chemical compound can be selected from peptides, polyketides, alkaloids, lactams and ethers such as tetrahydrofuran or any combinations of the above-mentioned chemical compounds.

Depending on the first valuable chemical compound to be produced, the respective first production genes encoding enzymes for the production of these first chemical compounds have to be introduced into the genetically enhanced cyanobacteria on the first extrachromosomal plasmid. For example, if the first chemical compound is ethanol, the first production gene encoding enzymes for ethanol production can be Pdc enzyme (pyruvate decarboxylase), Adh enzyme (alcohol dehydrogenase), or a AdhE enzyme (alcohol dehydrogenase E) which directly converts acetyl coenzyme A to ethanol. Pdc enzyme catalyzes the conversion of pyruvate to acetaldehyde, whereas the alcohol dehydrogenase, Adh enzyme, catalyzes the further conversion of acetaldehyde to the final first chemical compound ethanol. The Adh enzyme can, for example, be a Zn²⁺-dependent dehydrogenase such as AdhI from Zymomonas mobilis (ZmAdh) or the Adh enzyme from Synechocystis PCC6803 (SynAdh). Alternatively or in addition, the enzyme can also be an iron-dependent alcohol dehydrogenase (e.g. AdhII from Zymomonas mobilis). The Zn²⁺-dependent alcohol dehydrogenase can, for example, be an alcohol dehydrogenase enzyme having at least 60%, 70%, preferably 80% and most preferred 90% or even more than 90% sequence identity to the amino acid sequence of Zn²⁺ dependent Synechocystis Adh. Experiments have shown that in particular Synechocystis Alcohol dehydrogenase (slr1192) is able to ensure a high ethanol production in genetically enhanced cyanobacteria due to the fact that the forward reaction, the reduction of acetaldehyde to ethanol is much more preferred for Synechocystis Alcohol dehydrogenase enzyme than the unwanted back reaction from ethanol to acetaldehyde.

The AdhE is an iron-dependent, bifunctional enzyme containing a CoA-depending aldehyde dehydrogenase and an alcohol dehydrogenase activity. One characteristic of iron-dependent alcohol dehydrogenases (e.g. AdhE and AdhII) is the sensitivity to oxygen. In the case of the AdhE from E. coli a mutant was described that shows in contrast to the wild type also Adh activity under aerobic conditions. The site of the mutation was determined in the coding region at the codon position 568. The G to A nucleotide transition in this codon results in an amino acid exchange from glutamate to lysine (E568K). The E568K derivate of the E. coli AdhE is active both aerobically and anaerobically. [Holland-Staley et al., Aerobic activity of Escherichia coli alcohol dehydrogenase is determined by a single amino acid, J Bacteriol. 2000 November; 182(21):6049-54].

AdhE enzymes directly converting acetyl coenzyme A to ethanol can preferably be from a thermophilic source thereby conferring an enhanced degree of stability. The AdhE can be from Thermosynechococcus elongatus BP-1 or also can be a non-thermophilic AdhE enzyme from E. coli.

The pyruvate decarboxylase can for example be from Zymomonas mobilis, Zymobacter palmae or the yeast Saccharomyces cerevisiae. Regarding the nucleic acid sequences, protein sequences and properties of these ethanologenic enzymes, reference is made to the PCT patent application WO 2009/098089 A2, which is incorporated for this purpose.

Two other alcohols which are relatively widespread are propanol and butanol. Similar to ethanol, they can be produced by fermentation processes. The following enzymes are involved in isopropanol fermentation and can be encoded by first production genes located on the first extrachromosomal plasmid: acetyl-CoA acetyltransferase (EC:2.3.1.9), acetyl-CoA:acetoacetyl-CoA transferase (EC:2.8.3.8), acetoacetate decarboxylase (EC:4.1.1.4) and isopropanol dehydrogenase (EC:1.1.1.80).

The following enzymes are involved in isobutanol fermentation: acetolactate synthase (EC:2.2.1.6), acetolactate reductoisomerase (EC:1.1.1.86), 2,3-dihydroxy-3-methyl-butanoate dehydratase (EC:4.2.1.9), α-ketoisovalerate decarboxylase (EC:4.1.1.74), and alcohol dehydrogenase (EC:1.1.1.1).

In the case that ethylene is to be produced as a first valuable chemical compound, the at least one first production gene encodes an enzyme for ethylene formation, in particular the ethylene-forming enzyme 1-aminocyclopropane-1-carboxylate oxidase (EC 1.14.17.4), which catalyzes the last step of ethylene formation, the oxidation of 1-aminocyclopropane-1-carboxylic acid to ethylene. The substrate for the ethylene-forming enzyme is synthesized by the enzyme 1-aminocyclopropane-1-carboxylic acid synthase (EC 4.4.1.14) from the amino acid methionine.

If the first valuable chemical compound is an isoprenoid such as isoprene, the at least one first production gene encodes an enzyme such as isoprene synthase. Isoprene synthase (EC 4.2.3.27) catalyzes the chemical reaction from dimethylallyl diphosphate to isoprene and diphosphate.

Terpenes are a large and very diverse class of organic compounds, produced primarily by a wide variety of plants, particularly conifers. Terpenes are derived biosynthetically from units of isoprene and are major biosynthetic building blocks in nearly every living organism. For example, steroids are derivatives of the triterpene squalene. When terpenes are modified chemically, such as by oxidation or rearrangement of the carbon skeleton, the resulting compounds are generally referred to as terpenoids. Terpenes and terpenoids are the primary constituents of the essential oils for many types of plants and flowers. Examples of biosynthetic enzymes are farnesyl pyrophosphate synthase (EC 2.5.1.1), which catalyzes the reaction of dimethylallylpyrophosphate and isopentenyl pryrophosphate yielding farnesyl pyrophosphate. Another example is geranylgeranyl pyrophosphate synthase (EC 2.5.1.29), which catalyzes the reaction between transfarnesyl diphosphate and isopentenyl diphosphate yielding diphosphate and geranylgeranyl diphosphate.

Another example of first valuable chemical compounds are the so-called non-ribosomal peptides (NRP) and the polyketides (PK). These compounds are synthesized by plants, fungi and only a few bacteria such as actinomycetes, myxobacteria and cyanobacteria. They are a group of structurally diverse secondary metabolites and often possess bioactivities of high pharmacological relevance. Hybrids of non-ribosomal peptides and polyketides also exist, exhibiting both a peptide and a polyketide part. First production genes for the production of non-ribosomal peptides as the first chemical compounds are for example gene clusters encoding for non-ribosomal peptide synthesases (NRPS). NRPS are characteristic modular multidomain enzyme complexes encoded by modular non-ribosomal peptide synthases gene clusters. Examples for non-ribosomal peptide synthesases are Actinomycin Synthetase and Gramicidin Synthetase.

In general there are two distinct groups of polyketides (PK), the reduced polyketides of type I, the so-called macrolides and the aromatic polyketides of type II. Type I polyketides are synthesized by modular polyketide synthases (PKS), which are characteristic modular multidomain enzyme complexes encoded by modular PKS gene clusters. Examples for first production genes for the production of type I polyketides are the Rapamycin Synthase gene cluster and the Oleandomycin Synthase gene cluster. One example for a first production gene for type II polyketides is the Actinorhodin polyketide synthase gene cluster.

Examples for first production genes for the production of hydrids of polyketides and non-ribosomal peptides are the Microcystin Synthetase gene cluster, Microginin Synthetase gene cluster, Myxothiazole Synthetase gene cluster.

Further examples of first valuable chemical compounds are the alkaloids. Alkaloids are a compound group which is synthesized by plants. Alkaloids have highly complex chemical structures and pronounced pharmacological activities. Examples for biosynthetic enzymes for alkaloids which can be encoded by first production genes for the production of the chemical compound are strictosidine synthase, which catalyzes the stereoselective Pictet-Spengler reaction of tryptamine and secologanin to form 3a(S)-strictosidine. The primary importance of strictosidine is not only its precursor role for the biosynthetic pathway of ajmaline but also because it initiates all pathways leading to the entire monoterpene indol alkaloid family. Another example of an enzyme encoded by a first production gene is strictosidine glucosidase from the ajmaline biosynthetic pathway. This enzyme is able to activate strictosidine by deglycosylation thus generating an aglycon. This aglycon of strictosidine is the precursor for more than 2,000 monoterpenoid indol alkaloids.

Further examples of enzymes encoded by first production genes are:
(R,S)-3′-hydroxy-N-methylcoclaurine 4′-O-methyltransferase (4′OMT) central to the biosynthesis of most tetrahydrobenzylisoquinolin-derived alkaloids;
Berberine bridge enzyme (BBE) specific to the sanguinarine pathway;
(R,S)-reticuline 7-O-methyltransferase (7OMT) specific to laudanosine formation;
Salutaridinol 7-O-acetyltransferase (SalAT) and codeinone reductase that lead to morphine.

Vitamins, as further examples of first chemical compounds, are organic compounds that are essential nutrients for certain organisms and act mainly as cofactors in enzymatic reactions but can also have further importance, e.g. as anti oxidants in case of vitamin C. Vitamin C can be synthesized via the L-Ascorbic acid (L-AA) biosynthetic pathway from D-glucose in plants. The following enzymes are involved in vitamin C synthesis and can be encoded by first production genes:

Hexokinase, Glucose-6-phosphate isomerase, Mannose-6-phosphate isomerase, Phosphomannomutase, Mannose-1-phosphate guanylyltransferase, GDP-mannose-3,5-epimerase, GDP-L-galactose phosphorylase, L-Galactose 1-phosphate phosphatase, L-galactose dehydrogenase, L-galactono-1,4-lactone dehydrogenase.

Lactams are cyclic amides whereas the prefixes indicate how many carbon atoms (apart from the carbonyl moiety) are present in the ring: β-lactam (2 carbon atoms outside the carbonyl, 4 ring atoms in total), γ-lactam (3 and 5), δ-lactam (4 and 6). One example for a γ-lactam is Pyrrolidone, a colorless liquid which is used in industrial settings as a high-boiling, non-corrosive, polar solvent for a wide variety of applications. It is also an intermediate in the manufacture of polymers such as polyvinylpyrrolidone and polypyrrolidone.

Ethers are a class of organic compounds that contain an ether group—an oxygen atom connected to two alkyl or aryl groups—of general formula R—O—R. A well-known example is Tetrahydrofuran (THF), a colorless, water-miscible organic liquid. This heterocyclic compound is one of the most polar ethers with a wide liquid range, it is a useful solvent. Its main use, however, is as a precursor to polymers.

One example for the natural occurring ethers are the divinyl ether oxylipins. The main enzymes involved in their biosynthesis are the lipoxygenase and especially the divinyl ether synthase.

Alkanes (also known as saturated hydrocarbons) are chemical compounds that consist only of the elements carbon (C) and hydrogen (H) (i.e., hydrocarbons), wherein these atoms are linked together exclusively by single bonds (i.e., they are saturated compounds). Each carbon atom must have 4 bonds (either C—H or C—C bonds), and each hydrogen atom must be joined to a carbon atom (H—C bonds). The simplest possible alkane is methane, CH₄. There is no limit to the number of carbon atoms that can be linked together. Alkanes, observed throughout nature, are produced directly from fatty acid metabolites. A two-gene pathway widespread in cyanobacteria is responsible for alkane biosynthesis and can be included in the first extrachromosomal plasmid encoded by the first production genes. An acyl-ACP reductase (EC: 1.3.1.9) converts a fatty acyl-ACP into a fatty aldehyde that is subsequently converted into an alkane/alkene by an aldehyde decarbonylase (EC:4.1.99.5.).

Biopolymers such as polyhydroxyalkanoates or PHAs are linear polyesters produced in nature by bacterial fermentation of sugar or lipids. They are produced by the bacteria to store carbon and energy. The simplest and most commonly occurring form of PHA is the fermentative production of poly-3-hydroxybutyrate (P3HB) but many other polymers of this class are produced by a variety of organisms: these include poly-4-hydroxybutyrate (P4HB), polyhydroxyvalerate (PHV), polyhydroxyhexanoate (PHH), polyhydroxyoctanoate (PHO) and their copolymers. The main enzymes involved in PHA synthesis are as follows: For P3HB synthesis two molecules of acetyl-CoA were condensed by a β-ketothiolase (EC:2.3.1.9) to synthesize acetoacetyl-CoA, which is converted to (R)-3-hydroxybutyryl-CoA (3HBCoA) by NADPH-dependent acetoacetyl-CoA reductase (EC:1.1.1.36). The 3HBCoA is subsequently polymerized by poly(3-hydroxyalkanoate) synthase (EC:2.3.1.-) and converted to (P3HB).

About 100,000 metric tons of the natural fatty acids are consumed in the preparation of various fatty acid esters. The simple esters with lower chain alcohols (methyl-, ethyl-, n-propyl-, isopropyl- and butyl esters) are used as emollients in cosmetics and other personal care products and as lubricants. Esters of fatty acids with more complex alcohols, such as sorbitol, ethylene glycol, diethylene glycol and polyethylene glycol are consumed in foods, personal care, paper, water treatment, metal working fluids, rolling oils and synthetic lubricants. Fatty acids are typically present in the raw materials used for the production of biodiesel. A fatty acid ester (FAE) can be created by a transesterification reaction between fats or fatty acids and alcohols. The molecules in biodiesel are primarily fatty acid methyl esters FAMEs, usually obtained from vegetable oils by transesterification with methanol. The esterification of the ethanol with the acyl moieties of coenzyme A thioesters of fatty acids can be realized enzymatically by an unspecific long-chain-alcohol O-fatty-acyltransferase (EC 2.3.1.75) from Acinetobacter baylyi strain ADP1.

The genetically enhanced cyanobacterium can be selected from a group consisting of: Synechocystis, Synechococcus, Anabaena, Chroococcidiopsis, Chloreogloepsis, Cyanothece, Lyngbya, Phormidium, Nostoc, Spirulina, Arthrospira, Cyanobacterium, Trichodesmium, Leptolyngbya, Plectonema, Myxosarcina, Pleurocapsa, Oscillatoria, and Pseudanabaena.

According to another embodiment of the invention, the first gene essential or conditionally essential for the cyanobacterium is selected from genes encoding enzymes for the amino acid metabolism, nucleic acid metabolism, carbon metabolism, nitrogen, sulfur or phosphorus metabolism or from genes encoding essential RNA molecules such as ribosomal RNA and transfer RNA, resistance conferring genes or combinations thereof. Resistance conferring genes encode for proteins that are involved in the response of cells to stress conditions that would harm the cell vitality and lead to cell death, resp. Examples for such abiotic stress conditions are high and low temperature, high salinity, dryness, presence of toxic ions (e.g. metal ions) or compounds (e.g. biocides), high and low light intensity, nutrient limitations, radiation (e.g. UV or nuclear radiation). Examples of biotic stress conditions are phage or bacterial infections or predators. The resistance conferring genes are often up-regulated by the stressors itself. Their expression causes a stress-specific cell response allowing for the survival and fitness of the cell under the stress conditions. In contrast to that a cell that does not possess the resistance conferring gene for example due to a first gene inactivation will lose its ability to survive under these stress conditions. One non-limiting example of resistance conferring genes are the semi-metal or metal resistance conferring genes mentioned above. In particular, the following enzymes can be deactivated by a first gene inactivation and therefore can serve as marker genes:
amino acid metabolism:
argH (argininosuccinate lyase EC:4.3.2.1)
argG (argininosuccinate synthase EC:6.3.4.5)
hisB (imidazoleglycerol-phosphate dehydratase EC:4.2.1.19)
hisD (histidinol dehydrogenase EC:1.1.1.23)
aspC (aspartate aminotransferase EC:2.6.1.1)
lysA (diaminopimelate decarboxylase EC:4.1.1.20)
thrC (threonine synthase EC:4.2.3.1)
thrB (homoserine kinase EC:2.7.1.39)
trpB (tryptophan synthase EC:4.2.1.20)
proC (pyrroline-5-carboxylate reductase EC:1.5.1.2)
nucleic acid metabolism:
apt (adenine phosphoribosyltransferase EC:2.4.2.7)
adk (adenylate kinase EC:2.7.4.3)
gmk (guanylate kinase EC:2.7.4.8)
ndkR (nucleoside-diphosphate kinase EC:2.7.4.6)
nrdA/nrdB (ribonucleoside-diphosphate reductase alpha/beta EC:1.17.4.1)
purA (adenylosuccinate synthetase EC:6.3.4.4)
purB (adenylosuccinate lyase EC:4.3.2.2)
guaA (GMP synthase EC:6.3.5.2)
guaB (inosine 5-monophosphate dehydrogenase EC:1.1.1.205)
nitrogen metabolism:
narB (ferredoxin-nitrate reductase EC:1.7.7.2)
nirA (ferredoxin-nitrite reductase EC:1.7.7.1)
nrtABCD (nitrate ABC transporter)
narM (assembly factor of ferredoxin-nitrate reductase)
ureABC (urease alpha, beta and gamma subunit EC:3.5.1.5)
urtABCDE (urea ABC transporter)
ureDEFG (assembly factors of urease)
glnN/glnA (glutamine synthetase EC:6.3.1.2)
sulfur metabolism:
sat (sulfate adenylyltransferase EC:2.7.7.4)
cysC (adenylylsulfate kinase EC:2.7.1.25)
cysH (phosphoadenosine phosphosulfate reductase EC:1.8.4.8)
sir (ferredoxin-sulfite reductase EC:1.8.7.1)
cysM (cysteine synthase EC:2.5.1.47)
phosphorus metabolism:
sphS (phosphate sensor, two-component sensor histidine kinase)
sphR (response regulator of SphS)
pstS (phosphate-binding protein)
pstB (phosphate transport ATP-binding protein)
pstA/C (phosphate transport system permease protein)
sphX (periplasmic phosphate-binding protein of ABC transporter)
metabolism of cofactors and vitamins:
thiE (thiamine-phosphate pyrophosphorylase EC:2.5.1.3)
ribH (riboflavin synthase beta chain EC:2.5.1.-)
ribC (riboflavin synthase subunit alpha EC:2.5.1.9)
ribF (riboflavin kinase/FMN adenylyltransferase EC:2.7.1.26/2.7.7.2)
nadA (quinolinate synthase EC:2.5.1.72)
nadC (nicotinate-nucleotide pyrophosphorylase EC:2.4.2.19)
nadE (NAD synthetase EC:6.3.1.5)
panC (pantoate ligase/cytidylate kinase EC:6.3.2.1/2.7.4.14)
dfp (phosphopantothenoylcysteine decarboxylase/phosphopantothenate synthase (EC:4.1.1.36/6.3.2.5)
bioD (dithiobiotin synthetase EC:6.3.3.3)
bioB (biotin synthetase EC:2.8.1.6)
carbon metabolism:
gltA (citrate synthase EC:2.3.3.5)
icd (isocitrate dehydrogenase EC:1.1.1.41)
sucC (succinyl-CoA synthetase EC:6.2.1.5)
zwf (glucose-6-phosphate 1-dehydrogenase EC:1.1.1.49)
gnd (6-phosphogluconate dehydrogenase EC:1.1.1.44)
tktA (transketolase EC:2.2.1.1)
prk (phosphoribulokinase EC:2.7.1.19)
rbcLS (ribulose bisophosphate carboxylase EC:4.1.1.39)
pgk (phosphoglycerate kinase EC:2.7.2.3)
gap2 (glyceraldehyde-3-phosphate dehydrogenase EC:1.2.1.12)
genes encoding essential RNA molecules:
rrn16Sa (16S ribosomal RNA)
rrn5Sa (5S ribosomal RNA)
tRNA-Glu (transfer RNA for glutamate)
tRNA-Gln (transfer RNA for glutamine)
tRNA Ala (transfer RNA for alanine)
tRNA-Asp (transfer RNA for aspartate)
tRNA-Asn (transfer RNA for asparagine)
tRNA-Leu (transfer RNA for leucine)
tRNA-Val (transfer RNA for valine)
rnpB (RNA subunit of ribonuclease P)

In particular, the essential genes, which can be inactivated by a first gene inactivation can be selected from a group of genes, which were identified as being essential in a computational reconstruction of the primary metabolic network of Synechocystis sp. PCC 6803 (Knoop et al.: “The Metabolic Network of Synechocystis sp. PCC 6803: Systemic Properties of Autotrophic Growth”, Plant Physiology, September 2010, Vol. 154, pp. 410-422). The genes which were found to produce nonviable Synechocystis strains both in the in silico prediction and via in vivo studies are listed in Supplemental File S4 and Supplemental File S5.

In a further variant of the invention, the first essential or conditionally essential gene is preferably selected from a group consisting of pyrF, rbcLXS, leuB, narB, ziaRA, corRT and smtAB. A first gene inactivation in the rbcLXS operon for the large and small subunits and the chaperonin of the RubisCO (RubisCO: EC 4.1.1.39) is another example for a first gene inactivation in an essential gene for cyanobacteria. A first gene inactivation in the ziaRA gene is another example of a conditionally essential gene. The protein product of this gene (slr0798) confers resistance to zinc so that this gene becomes conditionally essential in the case that Zn²⁺ is supplemented into the growth medium in a concentration range of at least 5 μM, more preferred 5 to 30 μM.

In a further aspect of the invention, the genome of the genetically enhanced cyanobacterium harbors more than one copy of the first essential or conditionally essential gene and all copies of the first essential or conditionally essential gene carry at least one gene inactivation. This is particularly important in order to ensure that the first extrachromosomal plasmid harboring a copy of the first essential or conditionally essential gene is vital for the survival of the cyanobacteria in the growth medium. In the case that in the genome of the cyanobacterium some copies of the wild type first essential or conditionally essential gene remain, there is the risk that the first extrachromosomal plasmid is removed from the cyanobacterium because it is no longer vital for the survival of the cyanobacterium.

Cyanobacteria are known to be polyploid, harboring a large number of copies of the genome. For example one of the model organisms used in the patent application, Synechocystis sp. PCC 6803 has around 60 genome copies [Griese et al. “Ploidy in cyanobacteria”, FEMS Microbiology Letters, Volume 323, Issue 2, pages 124-131], October 2011. In general, cyanobacteria are known to contain approximately 10 genome copies per cell under laboratory growth conditions. In one preferred example of the invention the polyploid cyanobacterium is Synechocystis, in particular Synechocystis sp. PCC 6803.

As explained below, in particular for some embodiments of the present invention, the inventors surprisingly found out that it is very difficult or impossible to completely genetically segregate cyanobacteria harboring certain first gene inactivations in essential first genes, even if the essential factor in whose production the first essential gene is involved is present in the growth medium of the cyanobacteria. Incompletely segregated polyploid cyanobacteria include functional wild type copies of the first essential gene as well as further genome copies of the first essential gene inactivated by the first gene inactivation. Only genetically enhanced cyanobacteria, which are completely segregated and therefore only contain first essential genes inactivated by the first gene inactivation can be used in conjunction with the first extrachromosomal plasmid for a biocide-free expression system of the first chemical compound.

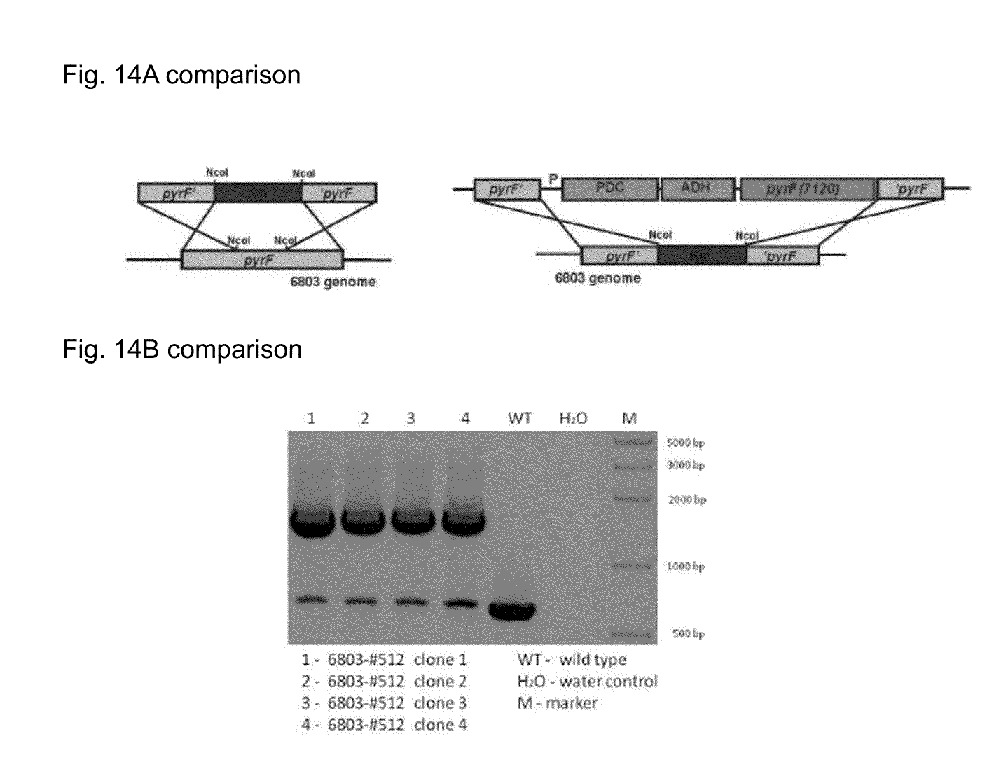

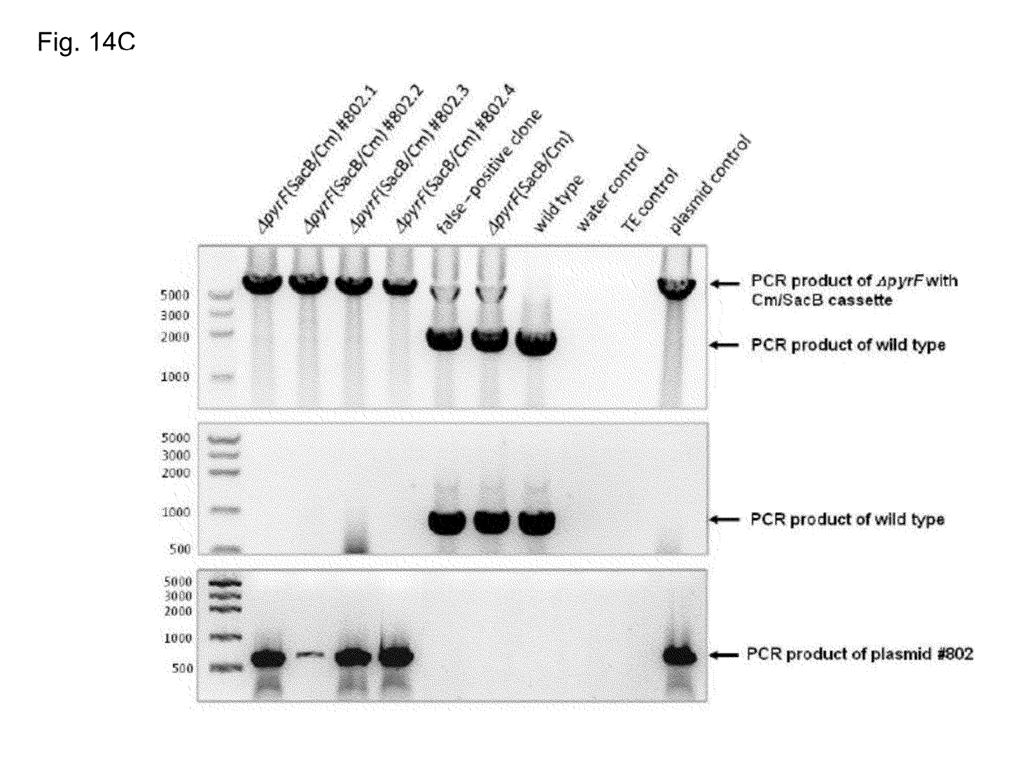

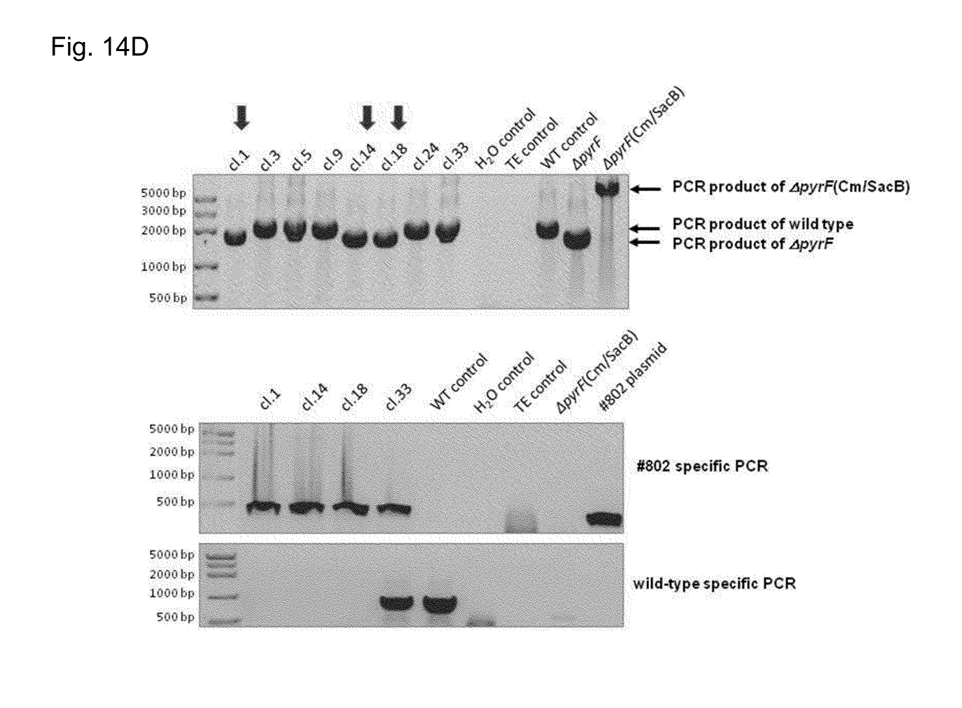

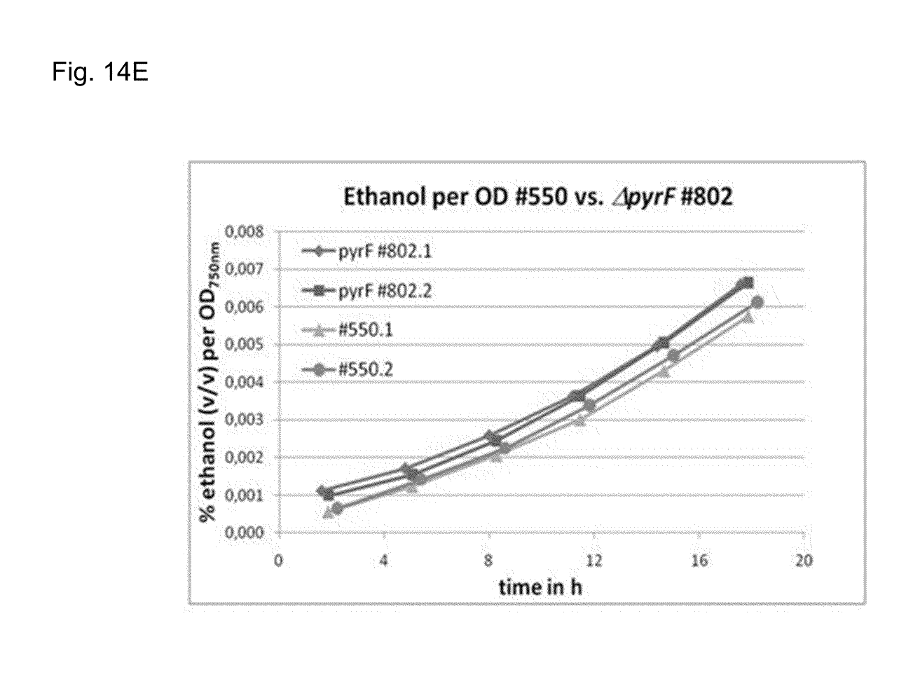

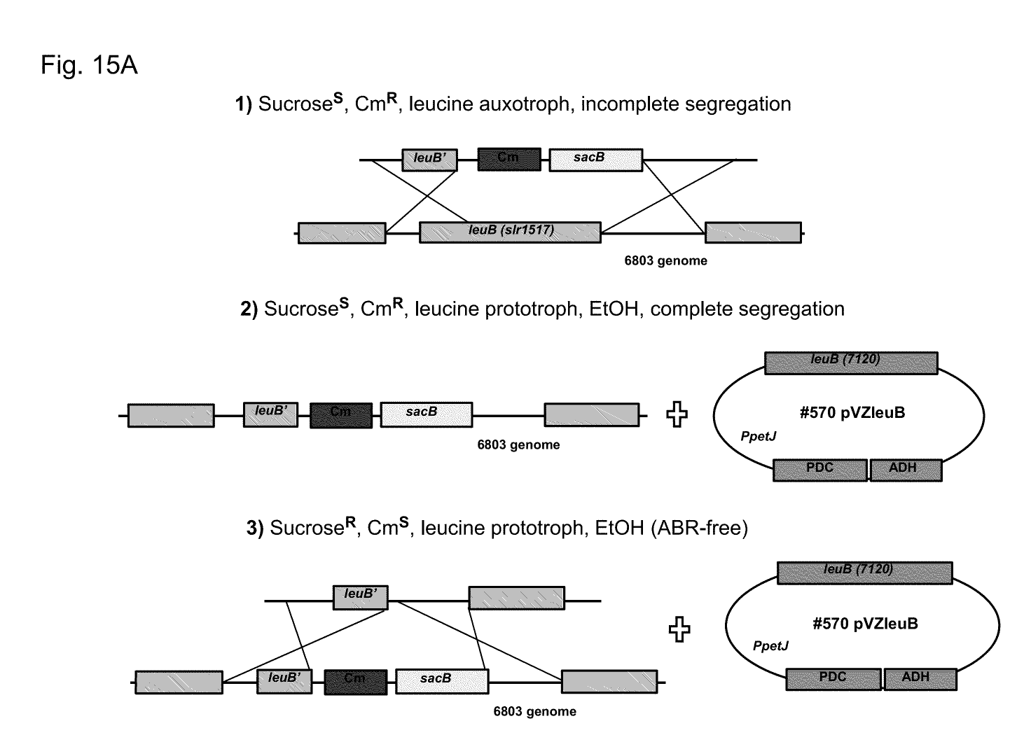

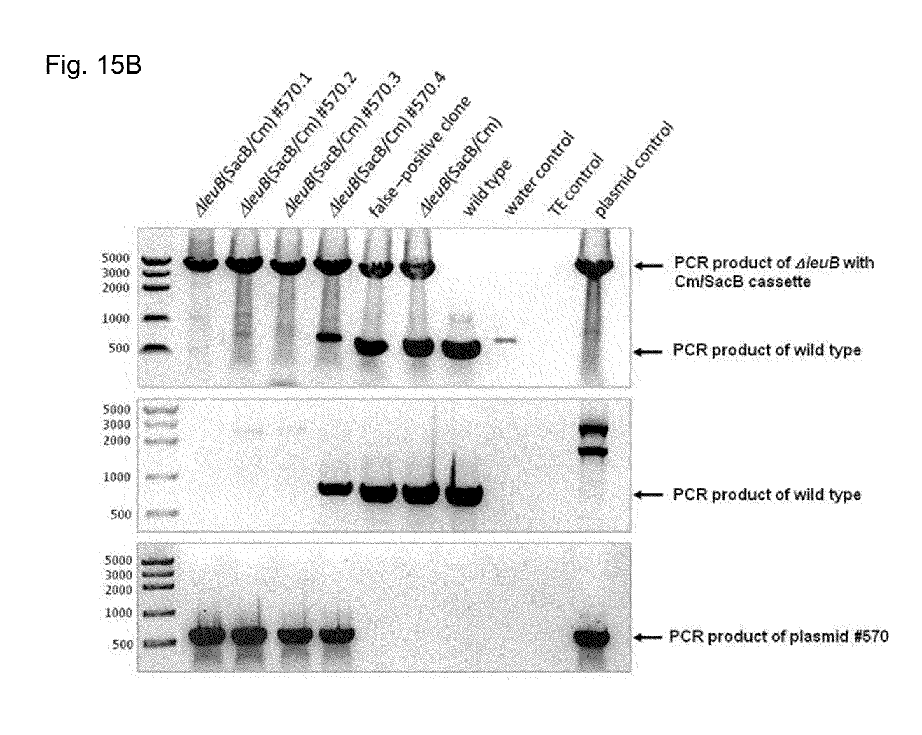

The inventors were for example not able to fully segregate cyanobacteria with first gene inactivations in the pyrF and leuB genes without first complementing these gene inactivations by the introduction of the first extrachromosomal plasmids (see for example FIGS. 14A to 14B). It was even not possible to obtain completely segregated genetically enhanced cyanobacteria with regard to the first gene inactivation lacking the first extrachromosomal plasmid, when the products in whose production pyrF and leuB are involved, uracil or leucine were supplemented into the growth medium.

According to another preferred embodiment of the invention, the first essential gene encodes either a first essential biocatalyst, such as an enzyme or ribozyme which is involved in the production of a first essential factor, which cannot promote complete genetic segregation of the cyanobacterium if present in the growth medium of the cyanobacterium, in the case that the cyanobacterium lacks a functional first essential gene. Alternatively the first essential gene encodes directly the first essential factor, which again cannot promote complete genetic segregation of the cyanobacterium if present in the growth medium of the cyanobacteria, in the case that the cyanobacterium lacks a functional first essential gene. Often, complete genetic segregation of recombinant host cells harboring a gene inactivation can be achieved by supplementing the factor, in whose production the inactivated gene is involved into the growth medium of the recombinant host cells. In these cases, the factor in the growth medium can be taken up by the recombinant host cells complementing for the autotrophy of the first factor.

The inventors surprisingly found out that in the case of a first gene inactivation in, for example, either the pyrF, or leuB gene, the first essential factor, either uracil or leucine cannot promote the complete genetic segregation of the cyanobacterial cells if present in the growth medium in the absence of the first chromosomal plasmid complementing for the auxotrophy of pyrF or leuB. The inventors were only able to identify incompletely segregated cyanobacteria, which both harbor pyrF and leuB genes with a first gene inactivation and also wild type fully functional pyrF and leuB genes. In this case, the inventors first had to introduce the first extrachromosomal plasmid into these incompletely segregated cyanobacteria before a full segregation of the cyanobacteria resulting in the genetically enhanced cyanobacteria harboring first gene inactivations in all genome copies of either pyrF or leuB was achieved.

Therefore, according to a further embodiment of the invention the first essential gene is involved in the production of a first essential factor, which is not able to promote genetic segregation of the cyanobacterium if present in the growth medium. This is particularly important for large-scale and long-term cultures of the cyanobacterial cells. In these long-term large-scale cultures cyanobacterial cells are prone to lysis, which releases all the intracellular components of the lysed cell into the growth medium. In the case that the first gene inactivation would affect a first essential factor which easily can be taken up by the cyanobacterial cell from the growth medium, there would be the risk in long-term cultures that the selection pressure to maintain the first extrachromosomal plasmid would be reduced due to the presence of the first essential factor in the growth medium. In the case that the first essential factor present in the growth medium cannot adequately promote the growth of the cyanobacterial cells, the selection pressure for the genetically enhanced cyanobacteria to maintain the first extrachromosomal plasmid is sustained even in large-scale long-term cultures. In general, the first essential or conditionally essential biocatalyst involved in the production of the first essential factor, which cannot promote complete genetic segregation of the cyanobacteria if present in the growth medium, can be selected from a group consisting of enzymes of the nucleic acid metabolism and enzymes of the amino acid metabolism.

Apart from pyrF and leuB, other first essential genes, encoding first essential biocatalysts (e. g. enzymes) such as the gene rbcLXS encoding RubisCO and a chaperone, can be used as selection marker proteins using the above-mentioned approach. RubisCO, if present in the growth medium cannot be taken up by the living cyanobacterial cells still present in the growth medium. Additional examples for first essential genes coding for essential proteins are petB/petD(slr0342/slr0343), which encode essential subunits of cytochrome b6f complex, psaC (ss10563) encoding an essential iron-sulfur cluster containing subunit of photosystem I and atpB/atpE (slr1329/slr1330) encoding an essential ATP synthase beta subunit and epsilon chain of CF(1).

In a further aspect of the invention, the first essential factor which cannot promote genetic segregation of the cyanobacterial cells with regard to the first gene inactivation if present in the growth medium cannot be taken up by the cyanobacterial cells. In general the uptake of compounds by a bacterial cell can either be passive, which requires no energy or can be an active transport into the bacterial cell, which requires energy, usually in the form of adenosine triphosphate (ATP). The passive transport of compounds into cells is either a diffusion such as the diffusion of oxygen and carbon dioxide or lipophilic compounds and the osmosis of water or is a facilitated diffusion, for example of glucose and other hydrophilic compounds. The facilitated diffusion is a carrier assisted transport which is mediated by specific transport proteins that are integrated into the cell membrane and are often highly selective for certain compounds. Both diffusion and facilitated diffusion are driven by the potential energy differences of a concentration gradient.

In contrast to that, the active transport of proteins often has to proceed against a concentration gradient. One example is the sodium potassium pump (see for example “Life: The Science of Biology, 4th edition by Sinauer Associates and W.H. Freeman). If the first essential factor cannot be taken up by the cyanobacterial cell, no passive or active transport of the first factor into the cyanobacterial cell will take place. One major example for a first essential factor which cannot be taken up by a cyanobacterial is RubisCO. RubisCO is known to be involved in the Calvin cycle for carbon fixation, which provides the building blocks for larger molecules such as glucose. However, supplementing the growth medium with glucose cannot complement for an inactivation in the genes encoding RubisCO.

The uptake of a first essential factor can be monitored in an axenic culture of cyanobacteria by including radioactive labels in the first essential factor and detecting the uptake of the radioactivity into the cells. First essential factors can for example be ¹⁴C-labelled such as ¹⁴C-labelled amino acids. In general, the procedure for monitoring the uptake of radiolabeled compounds into cyanobacterial cells is described in the publication Labarre et al.: “Genetic Analysis of Amino Acid Transport in the Facultatively Heterotrophic Cyanobacterium Synechocystis sp. Strain 6803”. J. Bacteriol., 169, p. 4668-4673 (1987), which is hereby incorporated by reference in its entirety.

The at least one first production gene for production of the first chemical compound can be under the transcriptional control of an inducible or constitutive promoter. In particular, an inducible promoter can be inducible by nutrient starvation and, for example light exposure. If the promoter is inducible by nutrient starvation a long-term large-scale culture of genetically enhanced cyanobacterial cells according to the invention can be automatically induced once the cyanobacterial culture has reached a certain density, which can lead to nutrient starvation, thereby simplifying the way the culture can be induced. In the uninduced state the cyanobacterial culture can first reach a high cell density before being automatically induced and growing into a state of nutrient starvation.

The inducible or constitutive promoter can be selected from a group consisting of PntcA, PnblA, PisiA, PpetJ, PpetE, PggpS, PpsbA2, PpsaA, PsigB, PlrtA, PhtpG, PnirA, PhspA, PclpB1, PhliB, Prbc, and PcrhC. Regarding the nucleic acid sequence and properties of these promoters, reference is made to the PCT patent application WO 2009/098089 A2, which is incorporated for this purpose.

Preferably, the inducible or constitutive promoters are selected from a group consisting of PnirA, PpetJ, PpetE, Prbc and combinations thereof. In particular, the inducible promoters of petJ, which is inducible by copper deprivation and the promoter of rbcLXS operon, which is a constitutive promoter, can be used as promoters controlling the transcription of first production genes. For example PpetJ can control the transcription of Pdc enzyme and Prbc can control the transcription of Adh enzyme in the case that the first production genes are for the production of ethanol. In these cases, high ethanol production rates can be reached which are comparable to conventional genetically enhanced cyanobacteria, which harbor extrachromosomal plasmids with antibiotic resistance conferring genes.

For example, an average ethanol production rate of 0.0164% (v/v) per day over a time period of 70 days (in day/night cycles) was reached in the case that the genetically enhanced cyanobacteria harbor a first gene inactivation in the leuB gene and also include a first extrachromosomal plasmid including a leuB gene under the transcriptional control of the petJ promoter and an additional Pdc and Synechocystis Adh encoding gene also under the transcriptional control of the petJ promoter. In general, the genetically enhanced cyanobacteria show an ethanol production rate of 0.01 to 0.05% (v/v) Ethanol per day/OD₇₅₀.

According to a further variant of the invention, the at least one production gene for the production of the first chemical compound comprises at least two first and second production genes coding for separate first and second production enzymes, which produce the first chemical compound. The first enzyme can for example be an enzyme producing an intermediate, which is then further converted by the second production enzyme into the first chemical compound. In the case of ethanol as a first chemical compound, the first and second production enzymes can be Pdc enzyme and Adh enzyme, wherein Pdc enzyme produces acetaldehyde from pyruvate and Adh enzyme further converts the acetaldehyde to ethanol.

Further, the first and second production genes can be under the transcriptional control of different promoters, which can be inducible or constitutive. In particular, the first production gene can be under the transcriptional control of an inducible promoter such as PpetJ, and the second production gene can be under the control of a constitutive promoter such as Prbc or Prbc*. In this case the first chemical compound such as ethanol can only be produced if the first production gene is induced.

Further, the inducible promoter for the at least one production gene, in particular for the gene directing the carbon flux away from the endogenous metabolism of the cyanobacterial host cell, encoding an enzyme with a catalytic activity not present in the wild type cyanobacterium, such as Pdc enzyme catalyzing the conversion of pyruvate to acetaldehyde, allows for the adjustment of the carbon flow towards the first chemical compound for example ethanol and biomass depending on the degree of induction. This might be necessary to optimize ethanol production depending on the overall carbon fixation (at lower carbon fixation per cell the induction level per cell can/should be lower, at higher carbon fixation per cell it is the opposite).

According to a further embodiment of the invention, the second production gene is located downstream of the first production gene with regard to the direction of transcription. In the case that the first and second production genes are transcriptionally controlled by different promoters, a transcription terminator sequence can be located downstream of the first production gene, upstream of the promoter controlling the second production gene in order to reduce the possibility that due to a read through event, transcription of the first production gene leads to a concomitant transcription of the second production gene. These terminator sequences can be oop from the lambda phage or the dsrA terminator sequence derived from the small non-coding RNA DsrA from E. coli [Lesnik E A, Sampath R, Levene H B, Henderson T J, McNeil J A, Ecker D J, “Prediction of rho-independent transcriptional terminators in Escherichia coli”, Nucleic Acids Res. 2001 Sep. 1; 29(17):3583-94].

According to another variant of the present invention, the first essential gene and the at least one production gene are grouped together in such a way on the first extrachromosomal plasmid that they are controlled by the same transcriptional regulators, so that a polycistronic mRNA is formed during transcription of such a gene operon, including both the first essential gene and the production gene. Furthermore both genes can be fused together in such a way that during translation a fusion protein harboring both the first biocatalyst encoded by the first essential gene and the enzyme encoded by the production gene is formed.

For such a functional coupling of transcription and translation of the first essential gene and the at least one production gene one has to assure that the at least one production gene is located upstream of the first essential gene. A protein fusion ensures a coupling of the translation of the first essential biocatalyst and the production enzymes for the first chemical compound.

According to yet another embodiment of the invention, the transcription of the first essential or conditionally essential gene and the at least one first production gene are separate. This means that the first essential or conditionally essential gene is controlled by a different promoter than the at least one first production gene. These promoters can be inducible promoters, which are inducible under different conditions or one promoter is an inducible promoter and the other promoter is a constitutive promoter. For example, the first essential or conditionally essential gene can be under the control of an inducible promoter, such as the heat shock promoter PhspA (Fang et al.:“Expression of the heat shock gene hsp16.6 and promoter analysis in the cyanobacterium, Synechocystis sp. PCC 6803”, Curr Microbiol. 2004 September; 49(3):192-8), which was found by the inventors to provide a moderate and relatively constant transcription level under the cultivation conditions used. The first production gene can be under the transcriptional control of another inducible promoter such as PpetJ. In this case, the cyanobacterial cells can be cultivated under conditions of induction of the first essential or conditionally essential gene but without inducing the first production gene. Only when the required cell density of the cyanobacterial cells is reached, production of the chemical compound can be induced for example by copper deprivation if PpetJ is used for controlling transcription of the first production gene.

In the case that the production genes for producing the first chemical compound comprise more than one gene, for example a Pdc enzyme encoding gene and an Adh enzyme encoding gene for ethanol production, only the gene coding for the enzyme, which directs the carbon flux away from the natural metabolic pathway of the cyanobacterium, i. e. the Pdc enzyme encoding gene can be put under the control of an inducible promoter such as PpetJ, whereas the second gene can be placed under the transcriptional control of a constitutive promoter such as Prbc*.

In a further aspect of the invention, the genetically enhanced cyanobacteria comprise a highly targeted first gene inactivation in the first essential or conditionally essential gene. This means that by the virtue of recombinant DNA technology a specific gene inactivation was introduced in the first essential or conditionally essential gene, which is in contrast to naturally occurring mutations or mutations introduced via random mutagenesis. Uncontrolled random mutations or naturally occurring mutations are often point mutations. In the case that a first extrachromosomal plasmid harboring the functional first essential or conditionally essential gene is introduced into such a cyanobacterium, there is the risk that a functional first essential gene can be reintroduced into the genome of the cyanobacterium again for example, via homologous recombination or a spontaneous reversion of the initial point mutation. In contrast to that, certain embodiments of the present invention provide genetically enhanced cyanobacteria wherein a large part of the wild type first essential or conditionally essential gene, such as 40%, 60% or more preferred up to 100% are deleted. Certain embodiments of this invention provide deletions of up to 40% in for example the pyrF and leuB genes of Synechocystis sp. PCC 6803. Further variants provide deletions of 98.6% of the narB gene and a complete deletion of the ziaRA, corRT and smtAB genes respectively, which reduces the risk of an unwanted homologous recombination leading to a reconstitution of a fully functional first essential gene.

In a more preferred embodiment of the invention, the first gene inactivation comprises not just a partial deletion of the first essential gene, but rather a complete deletion of the first essential gene. In this case, a reintroduction of a functional first essential gene into the genome of the genetically enhanced cyanobacterium via homologous recombination is no longer possible, because homologous recombination would require that at least a part of the genomic sequence of the first essential gene still would be present in the genome of the genetically enhanced cyanobacterium.

Beside the disadvantage of uncontrolled random mutations or naturally occurring mutations in regard to the risk of an unwanted homologous recombination leading to a reconstitution of a fully functional first essential gene there are more disadvantages in connection with the undirected way of the mutation.

Due to the untargeted nature of random mutations, it is for instance very unlikely that mutations caused by random mutations or naturally occurring mutations only inactivate the first essential or conditionally essential gene but do not affect other genes, promoters or regulatory elements in parallel. Therefore one can't assure that the cell is not negatively affected by further random mutations or naturally occurring mutations whereas for a targeted gene inactivation, the inactivation is restricted to the first essential or conditionally essential gene.

Another disadvantage of the undirected way of random mutations or naturally occurring mutations is that compared to directed mutagenesis it will be much more difficult to obtain a cyanobacterial strain with several inactivations in essential or conditionally essential genes of one organism and again it will be even more likely to affect also other genes, promoters or regulatory elements in parallel which negatively affect the cell.

Another disadvantage of the undirected way of random mutations or naturally occurring mutations is that in contrast to directed mutagenesis it can't be reproduced in exactly the same way. That means once the cell line is with an inactivation in one or more essential gene is selected it won't be possible to create another cell line with exactly the same genotype.

Another aspect of the invention is directed to a genetically enhanced cyanobacterium, which further comprises:
a second gene inactivation in a second essential or conditionally essential gene in the genome of the cyanobacterium, wherein the second essential or conditionally essential gene is different from the first essential or conditionally essential gene, and
at least one second production gene different from the first production gene, wherein
the second essential or conditionally essential gene and the second production gene are included on either the first extrachromosomal plasmid or on a second extrachromosomal plasmid.

The second extrachromosomal plasmid can be different from the first extrachromosomal plasmid. Alternatively, the second essential or conditionally essential gene can also be harbored on the first extrachromosomal plasmid. This would further increase the need to maintain the first extra-chromosomal plasmid and therefore would enhance the plasmid stability if two essential genes are located on the first extrachromosomal plasmid. In addition, the second essential or conditionally essential gene and the second production gene can also be located on the first extrachromosomal plasmid, if only one type of extrachromosomal plasmid can be introduced into the cyanobacterium or in the case that only one self-replicating plasmid can be replicated by the cyanobacterium. For the same reason of increasing plasmid stability, the at least one second production gene different from the first production gene can also be harbored on the first extrachromosomal plasmid.

Similarly to the first production gene the second production gene can also encode an enzyme for the production of a second chemical compound, which can be selected from a group consisting of alcohols, alkanes and alkenes, polyhydroxyalkanoates, e.g. PHB, fatty acids, fatty acid esters, carboxylic acids (such as amino acids), terpenes and terpenoids, peptides, polyketides, alkaloids, lactams, such as pyrrolidone, and ethers, such as THF or any combinations thereof. By introducing a second production gene different from the first production gene into the genetically enhanced cyanobacterium, the cyanobacterium will be able to produce a larger variety of first and second valuable chemical compounds.

In addition to or alternatively, the second production gene can also encode an endogenous enzyme of the cyanobacteria, wherein the expression of the endogenous enzyme results in an increased rate of production of the first chemical compound compared to the respective cyanobacterium harboring the first production gene, but lacking the second production gene.

The endogenous enzyme of the cyanobacterium which is encoded by the second production gene can, for example, be a gene which directs the metabolic flux of carbon which, in particular, is produced by photosynthesis in the photoautotrophic cyanobacteria towards the enzyme encoded by the first production gene, so that a higher production rate of the first chemical compound can be observed in comparison to the genetically enhanced cyanobacteria lacking the second production gene. In particular, the second production gene can code for an endogenous enzyme involved in the production of a substrate used by the enzyme encoded by the first production gene. Alternatively the enzyme encoded by the second production gene can also be involved in the synthesis of a precursor molecule for the substrate used by the enzyme encoded by the first production gene.

The endogenous enzyme can be an enzyme which is also present in the wild type cyanobacterium or it can be a homologous exogenous enzyme, which exhibits a high degree of sequence identity to the endogenous enzyme of the cyanobacterium, and shows the same enzymatic activity as the endogenous enzyme. For example, it is possible to overexpress enzymes of the glycolytic pathway such as pyruvate kinase, enolase and phosphoglycerate mutase from different sources such as E. coli and Zymomonas mobilis in Synechocystis cyanobacteria.

The endogenous enzymes encoded by the second production gene can, for example, be selected from a group consisting of: phosphoglycerate mutase, enolase, pyruvate kinase, ribulose-1,5-bisphosphate carboxylase/oxygenase (RubisCO), malic enzyme, phosphoenolpyruvate (PEP) carboxylase, malic enzyme, fbpI (slr2094) fructose-1,6-/sedoheptulose-1,7-bisphosphatase, tktA (sll1070) transketolase and malate dehydrogenase. Concerning the further properties, nucleic acid and protein sequences of these enzymes, reference is made to the PCT patent application WO 2009/098089 A2. The enzymes ribulose-1,5-bisphosphate carboxylase/oxygenase (RubisCO), fructose-1,6-/sedoheptulose-1,7-bisphosphatase and transketolase can lead to a higher CO₂ fixation rate and therefore to a higher flux of the carbon fixed via photosynthesis towards the production of the first chemical compound.

Furthermore, the genetically enhanced cyanobacterium can comprise first and, if present, also second extrachromosomal plasmids which are replication competent. This means that the first and second extrachromosomal plasmid contain an origin of replication and are able to replicate autonomously within the genetically enhanced cyanobacterium. The replication competent first extrachromosomal plasmids can be so-called high copy or low copy number plasmids which are either present in a high number or a low number within the genetically enhanced cyanobacterium. The cyanobacterium Synechococcus PCC 7002 is known to contain six endogenous plasmids having different numbers of copies in the cyanobacterial cell (Xu et al.: “Expression of genes in cyanobacteria: Adaption of Endogenous Plasmids as platforms for High-Level gene Expression in Synechococcus PCC 7002”, Photosynthesis Research Protocols, Methods in Molecular Biology, 684, pages 273 to 293 (2011)). The endogenous plasmid pAQ1 is present in a number of 50 copies per cell (high-copy), the plasmid pAQ3 with 27 copies, the plasmid pAQ4 with 15 copies and the plasmid pAQ5 with 10 copies per cell (low-copy). These endogenous plasmids can in principle also be used as integration platform for the essential or conditionally essential genes as well as for the production genes. These genes can for example be integrated into the endogenous cyanobacterial plasmids via homologous recombination. It is also possible to create so called shuttle vectors using the backbones of such plasmids and combine those with backbones of self-replicating E.coli vectors. The great advantage of incorporating the first production genes for producing the first chemicals compounds and the essential or conditionally essential genes into endogenous extrachromosomal plasmids of the cyanobacterium is that by the choice of the endogenous plasmid used for integration the number of copies of these genes in the cyanobacterium can easily be controlled, depending on the copy number of the specific endogenous plasmid that is used for that purpose in the cyanobacterium. For example, a higher number of copies of these genes can be achieved via integration of these genes into the plasmid pAQ3 in comparison to integration into the plasmid pAQ4 with a lower number of copies in the cell. The number of gene copies on the exogenous extrachromosomal plasmids in the genetically enhanced cyanobacteria, for example pVZ (RSF1010 based) plasmids cannot be controlled in such a way as for the endogenous plasmids.

The invention also encompasses a method for producing a first chemical compound comprising the method steps of:
A) Culturing a genetically enhanced cyanobacterium as described above in the absence of a biocide, the cyanobacterium producing the first chemical compound, and
B) Recovering the first chemical compound.

Due to the above-mentioned complementation strategy wherein the first extrachromosomal plasmid harbors the first essential or conditionally essential gene, the genetically enhanced cyanobacteria can be cultured for a long time without the need to use any biocides. Furthermore, the first valuable chemical compound can either be produced and accumulated within the cyanobacterial cells or can be secreted into the growth medium so that the first chemical compound can either be recovered from the growth medium or via processing, such as opening the cyanobacterial cells in order to recover the first chemical compound inside the cells.

Owing to the fact that cyanobacteria are phototrophic organisms during step A) the genetically enhanced cyanobacteria can preferably be subjected to light, such as sunlight and to CO₂ in order to increase the photosynthesis rate of the cyanobacterial cells. In this case the photosynthetic capacity of the cyanobacterial cells is used in order to produce the first chemical compound.

In the case that the first gene inactivation affects a first conditionally essential gene, the cyanobacteria are cultured in method step A) under a condition rendering the first conditionally essential gene an essential gene. For example in the case that the first gene inactivation affects the narB gene, the growth medium should not contain ammonia or nitrite as alternative nitrogen sources, so that genetically enhanced cyanobacteria harboring the first gene inactivation, but lacking the first extrachromosomal plasmid complementing for this first gene inactivation are not able grow in this medium. In this case only genetically enhanced cyanobacterium harboring both the first gene inactivation as well as the first extrachromosomal plasmid are able to grow in such a growth medium.

In a preferred variant of the method of the invention, the method is for the production of ethanol and the cyanobacteria are cultured harboring a first production gene encoding at least one enzyme for ethanol production, such as the already above-mentioned Adh, Pdc and AdhE enzymes or combinations thereof. In particular, ethanol producing genetically enhanced cyanobacteria can comprise a combination of Pdc enzyme and Adh enzyme or can only comprise AdhE enzyme directly converting acetyl coenzyme A into ethanol or also can just include Pdc enzyme, which converts pyruvate to acetaldehyde. In this case the endogenous Adh enzyme of the genetically enhanced cyanobacterium, for example, Synechocystis, can be sufficient in order to ensure a high ethanol production rate.

The genetically enhanced cyanobacteria according to some embodiments of this invention can be produced by a method comprising the method steps of:
i) transforming the cyanobacterium by introducing a first gene inactivation into a first essential or conditionally essential gene of the cyanobacterium, and
ii) introducing the first extrachromosomal plasmid harboring the first essential or conditionally essential gene and the at least one first production gene into the genetically enhanced cyanobacterium.

In principle, the first gene inactivation can comprise any suitable method in order to inactivate or even reduce the activity of the first essential or conditionally essential gene. For example, the promoter sequence controlling the transcription of the first essential or conditionally essential gene can be changed in order to reduce or completely eliminate the production rate of RNA molecules, such as messenger RNA molecules encoding the first essential or conditionally essential biocatalyst. Other methods of performing the first gene inactivation include deleting at least a part, preferably the whole genomic sequence of the first essential or conditionally essential gene. In particular, at least a part or the complete first essential or conditionally essential gene can be replaced with a recombinant nucleic acid sequence which does not contain the first essential or conditionally essential gene or parts thereof. In one embodiment of the method of the invention, if the gene to be inactivated is an essential gene, the first factor, in whose production the first essential gene is involved, needs to be present in the growth medium of the cyanobacteria at least during method step i) in order to achieve a complete segregation. In the case that the first gene to be inactivated is a conditionally essential gene, the cyanobacteria need to be cultured under conditions wherein the conditionally essential gene is not essential in order to obtain a complete segregation.

A further aspect of the invention is directed to a method for producing the genetically enhanced cyanobacteria, wherein the method step i) comprises the following substeps:
i1) transforming the cyanobacteria with a first recombinant nucleic acid sequence, wherein the nucleic acid sequence comprises a first selectable gene conferring resistance to a selectable marker and a second counterselectable gene conferring sensitivity to a counterselectable marker;
i2) selecting for transformed cyanobacteria by subjecting the cyanobacteria to the selectable marker;
i3) transforming the cyanobacteria obtained from step i2) with a second recombinant nucleic acid sequence lacking the first selectable and second counterselectable gene by replacing at least a part of the first recombinant nucleic acid sequence, thereby creating transformed cyanobacteria lacking a functional first selectable and functional second counterselectable gene;
i4) selecting for transformed cyanobacteria from step i3) via subjecting the cyanobacteria to the second counterselectable marker;
   and wherein during method step ii) the first extrachromosomal plasmid is introduced into the cyanobacteria obtained from step i4).

By applying this method, a general three-step method can be used wherein in a first step a first recombinant nucleic acid is introduced into the first essential or conditionally essential gene, thereby causing a gene disruption leading to the first gene inactivation (method step i1). In the main second step, i3), a second recombinant nucleic acid sequence lacking both the first selectable and the second counterselectable gene is introduced into at least a part of the first recombinant nucleic acid sequence thereby causing gene inactivations in the first selectable and second counterselectable gene. The main task of this substep i3) is to produce genetically enhanced cyanobacteria, which lack both, the first selectable gene, which can be a biocide gene and also the second counterselectable gene. Both genes are therefore only temporarily present in the cyanobacteria during the course of generating the final genetically enhanced cyanobacteria. In the last main step ii) the first extrachromosomal plasmid is introduced into the cyanobacteria, thereby producing the final genetically enhanced cyanobacteria of the invention.

The additional substeps i2) and i4) are carried out in order to select for the respective transformed cyanobacteria obtained in the transformation steps i1) and i3). In particular, during substep i2) the transformed cyanobacteria of substep i1) are selected for over the untransformed cyanobacteria by subjecting the cyanobacteria to the selectable marker, which for example can be a biocide marker such as an antibiotic like Chloramphenicol, kanamycin or gentamycin. In the other step, i4), transformed cyanobacteria from step i3) are selected for over the untransformed cyanobacteria from step i3) by subjecting the cyanobacteria to the counterselectable marker. In this case untransformed cyanobacteria from step i3), which still harbor the first recombinant nucleic acid sequence including at least the second counterselectable gene, will be killed since this gene confers a sensitivity to the counterselectable marker. The counterselectable gene can be selected from a group consisting of different genes, in particular including sacB, tetAR, rpsL, pheS, thyA, lacy, gata-1, and ccdB or combinations thereof. In particular all counterselectable markers described in the publication “Counterselectable Markers: Untapped Tools for Bacterial Genetics and Pathogenesis”, Infection and Immunity, 1998, pages 4011 to 4017, can be used. For example the sacB gene encodes the enzyme levansucrase from Bacillus subtilis that confers sucrose sensitivity on gram-negative bacteria such as cyanobacteria. In this case sucrose can be used as a counterselectable marker in order to select for cyanobacteria, which have lost the first exogenous nucleic acid sequence during the transformation substep i3). Alternatively, rps12, which is a gene encoding an s12 ribosomal protein subunit which, due to point mutations, confers resistance to streptomycin, can be used. If streptomycin-resistant cyanobacteria are transformed with a dominant wild type rps12 allele the genetically enhanced cyanobacteria will be sensitive to streptomycin in contrast to the untransformed cyanobacteria, so that streptomycin can be used as a counterselectable marker.

In particular, in the substeps i1) and i3) the first and second recombinant nucleic acid sequences can be introduced into the cyanobacteria via homologous recombination. This requires that the first recombinant nucleic acid sequence has nucleic acid sequences homologous to the first essential or conditionally essential gene to be inactivated or with nucleic acid sequences neighboring the first essential gene in the genome of the cyanobacterium. These homologous sequences are positioned at the 5′- and 3′-end of the first recombinant nucleic acid sequence and flank the first selectable gene and the second counterselectable gene. Similarly, the second recombinant nucleic acid sequence has to include 5′- and 3′-sequences which are homologous at least to parts of the nucleic acid sequence of the first recombinant nucleic acid.

Furthermore, polyploid cyanobacteria containing more than one copy of their genomes can be used in the methods of the invention. In this case, it is even possible to inactivate via the first gene inactivation first essential genes, which either encode first essential factors or which are involved in the production of first essential factors, which cannot promote the complete genetic segregation of cyanobacteria regarding a gene inactivation in the first gene when the first factor is supplemented into the growth media of these cyanobacteria.

The inventors found out that the above described method for generating a first gene inactivation sometimes cannot lead to complete genetic segregation with regard to gene inactivations in essential genes. In this case, a method of producing genetically enhanced cyanobacteria has to be employed, which is an alternative to the above mentioned method including the method steps i1) to i4). During the course of this alternative method, the first essential factor does not need to be present in the growth medium of the cyanobacteria, since it does not promote the genetic segregation. This alternative method includes the method step i) wherein in a first substep i′1) first gene inactivations are created in not all copies of the first essential gene by replacing at least parts of the first essential gene by the first recombinant nucleic acid, which means that only a part of the gene copies of the first essential gene present in the polyploid cyanobacterium are inactivated by the first gene inactivations, whereas another part of the copies of the first essential gene are retained as wild type copies. During the following method step i′2) a selection for the partially segregated cyanobacteria is carried out by subjecting the cyanobacteria from step i′1) to the first selectable marker. In particular, the concentration of the first selectable marker can be increased in a stepwise manner during method step i′2). In the subsequent second method step ii) the first extrachromosomal plasmid also harboring this first essential gene is introduced into the cyanobacterium in the presence of the first selectable marker in a concentration equal to or higher than the concentration employed during method step i′2). Subsequently, the cyanobacteria obtained from step ii) are subjected to a higher concentration of the first selectable marker during a method step i′3) in comparison to the concentration used in step i′2) above in order to obtain fully segregated cyanobacteria only containing the first recombinant nucleic acid, but no functional wild type copies of the first essential gene, anymore. Owing to the presence of the first extrachromosomal plasmid, all copies of the first essential gene can now be inactivated via the first gene inactivation since the first extrachromosomal plasmid complements for the first gene inactivation. In a further method step i′4) the first recombinant nucleic acid is replaced by the second recombinant nucleic acid and the cyanobacteria only containing the second, but not the first recombinant nucleic acid are selected for, by subjecting the cells to the second counterselectable marker in a method step i′5).

As already explained above, this method has the great advantage that even when culturing the genetically enhanced cyanobacteria in a high cell density and long-term cultures, the selection pressure to maintain the first extrachromosomal plasmid remains high even if lysed cells release the first essential factor into the growth medium. This is due to the surprising finding that the first essential factor present in the growth medium cannot promote the complete genetic segregation of the cells in the absence of the first extrachromosomal plasmid. Examples for biocatalysts for the production of such a first essential factor are for example pyrF, coding for the above-mentioned enzyme of the uracil synthesis pathway, leuB coding for an enzyme of the leucine synthesis pathway and rbcLXS operon, coding for RubisCO enzyme and the chaperonin.

In the following certain embodiments of the invention will be explained in more detail with reference to experimental results and figures.

The extrachromosomal plasmids used for the generation of the genetically enhanced cyanobacteria according to various embodiments of the invention as well as for the generation of conventional reference strains are shown in the following figures:

### BRIEF DESCRIPTION OF THE FIGURES AND SEQUENCES

In the following, among others, plasmid maps and sequence listings will be presented. The reading direction of the genes annotated in the sequence listing is indicated by the direction of the arrows in the respective plasmid maps. The so-called platforms present in some of the plasmids are 5′ and 3′ flanking nucleotide sequences which are neighboring sequences to endogenous genes which are to be deleted via homologous recombination. The sequences which are flanked by these platforms were incorporated depending on the plasmid either into the cyanobacterial chromosome or into the endogenous extrachromosomal plasmids of the cyanobacterium via homologous recombination.

The calculation of the graphs depicting the ethanol production rates was done with the program Microsoft Excel from the Microsoft Office 12 package. From the values depicting the ethanol production rates at certain points of time of cultivation, the actual ethanol production rate was calculated via linear regression using the “add trendline” command of Microsoft Excel.

### BRIEF DESCRIPTION OF THE DRAWINGS

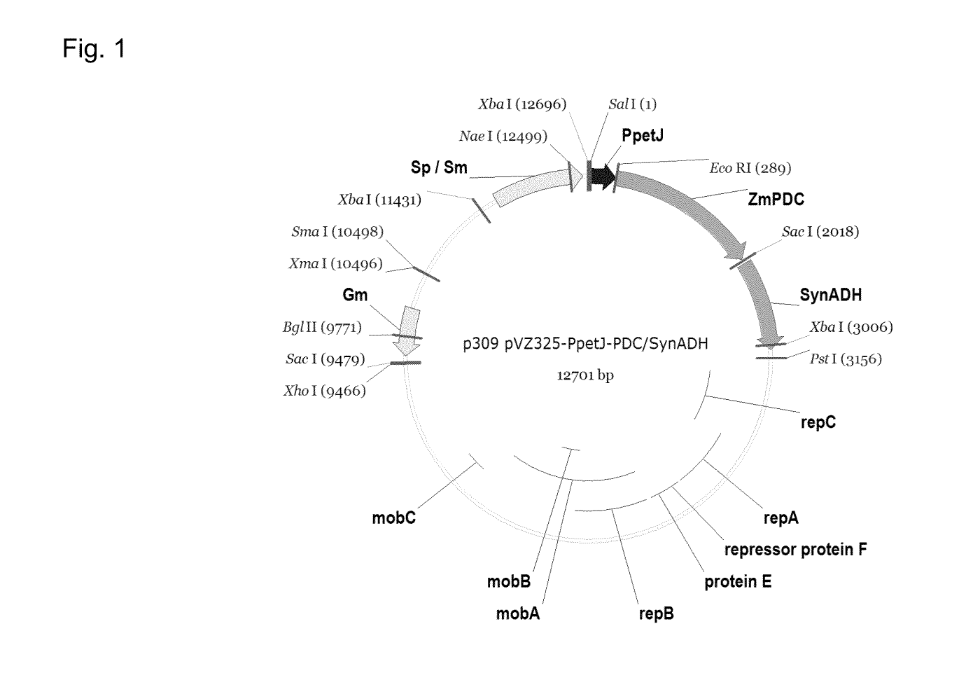

FIG. 1 denotes a schematic plasmid map of the plasmid #309 for the generation of a cyanobacterium harbouring genes for the ethanologenic enzymes ZmPdc and SynAdh controlled by the promoter PpetJ, a Gentamycin conferring resistance cassette (Gm) and a Spectinomycin/Streptomycin resistance conferring gene (Sp/Sm).

SEQ ID NO. 1 is the DNA sequence of the plasmid #309 indicating the start and end points of various genes encoded on this plasmid. This plasmid contains from nucleotides 7873 to 8154 the gene mobC, from nucleotides 5548 to 7674 the gene mobA, from nucleotides 6516 to 6926 the gene mob, from nucleotides 5545 . . . 6516 the gene repB, from nucleotides 5272 to 5484 the gene for protein\E, from nucleotides 5064 to 5270 the gene for repressor\protein\F, from nucleotides 4198 to 5034 the gene repA, from nucleotides 3357. to 4208 the gene repC, from nucleotides 2049 to 3059 the gene for SynADH (slr1192), from nucleotides 1 to 285 the copper dependent promoter PpetJ, from nucleotides 286 to 1995 the gene coding for ZmPDC (pyruvate decarboxylase from Zymomonas mobilis), from nucleotides 11632 to 12640 the Streptomicin/Spectinomicin resistance cassette, and from nucleotides 9555 to 10088 the Gentamicin resistance cassette.

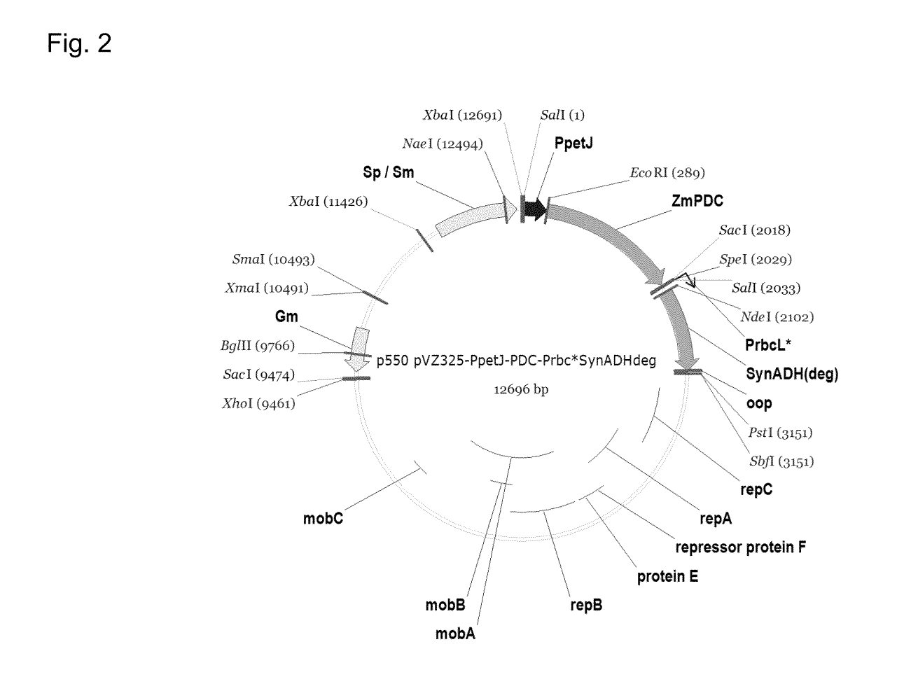

FIG. 2 shows the plasmid map of the plasmid #550, used to generate cyanobacteria with an antibiotic resistance marker for Gentamycin (Gm) and Spectinomycin/Streptomycin (Sp/Sm). The ethanologenic genes are under the transcriptional control of PpetJ for ZmPdc and under the transcriptional control of Prbc* for SynAdh_{deg}.

SEQ ID NO. 2 depicts the DNA sequence of the plasmid #550 indicating the start and end points of various genes encoded on this plasmid. This plasmid contains from nucleotides 3114 to 3144 the gene for SynADH(deg), the codon degenerated form of gene slr1192 coding for Zn2+ dependent ADH of Synechocystis 6803, from nucleotides 2037 to 2101 the modified core promoter element PrbcL* (from −35 to ATG), from nucleotides 9550 to 10083 the Gentamicin resistance cassette (Gm), from nucleotides 11627 to 12635 the Streptomicin/Spectinomicin resistance cassette (Sm), from nucleotides 1 to 285 PpetJ, from nucleotides 3352 to 4203 the gene repC, from nucleotides 4193 to 5029 the gene for repressor\protein\F, from nucleotides 5267 to 5479 the gene for protein\E, from nucleotides 5540 to 6511 the gene repB, from nucleotides 6511 to 6921 the gene mob, from nucleotides 5543 to 7669 the gene mobA, from nucleotides 7868 to 8149 the gene mobC, and from nucleotides the gene coding for ZmPDC.

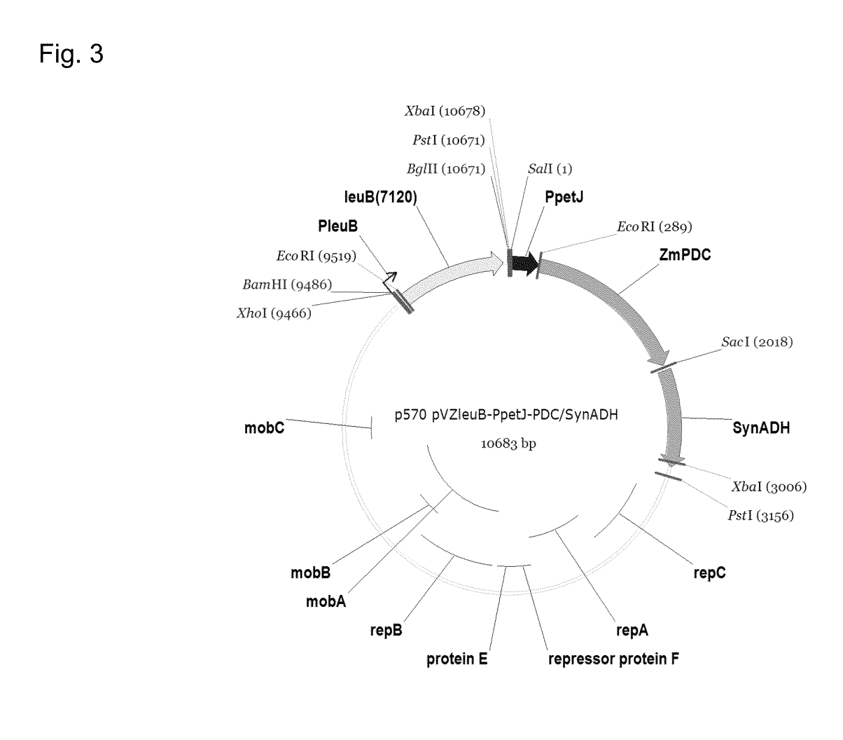

FIG. 3 shows the plasmid map of the plasmid #570, which can complement for a first gene inactivation in the leuB gene and harbours the leuB gene of Anabaena sp. PCC7120 in addition to the ethanologenic genes encoding ZmPdc and SynAdh, which are both under the transcriptional control of PpetJ.

SEQ ID NO. 3 depicts the DNA sequence of the plasmid #570 indicating the start and end points of various genes encoded on this plasmid. This plasmid contains from nucleotides 9528 to 10616 the gene leuB(7120), leuB (alr1313) from Anabaena PCC7120 for selection in E.coli KC8 and Syn6803 leuB-knockout, from nucleotides 286 to 1995 the gene coding ZmPDC, from nucleotides 1 to 285 PpetJ, from nucleotides 2049 to 3059 the gene coding for SynADH, from nucleotides 3357 to 4208 the gene repC, from nucleotides 4198 to 5034 the gene repA, from nucleotides 5064 to 5270 the gene coding for repressor\protein\F, from nucleotides 5272 to 5484 the gene for protein\E, from nucleotides 5545 to 6516 the gene repB, from nucleotides 6516 to 6926 the gene mob, from nucleotides 5548 to 7674 the gene mobA, from nucleotides 7873 to 8154 the gene mobC, and from nucleotides 9466 to 9527 PleuB, the leuB promoter from Anabaene PCC7120.

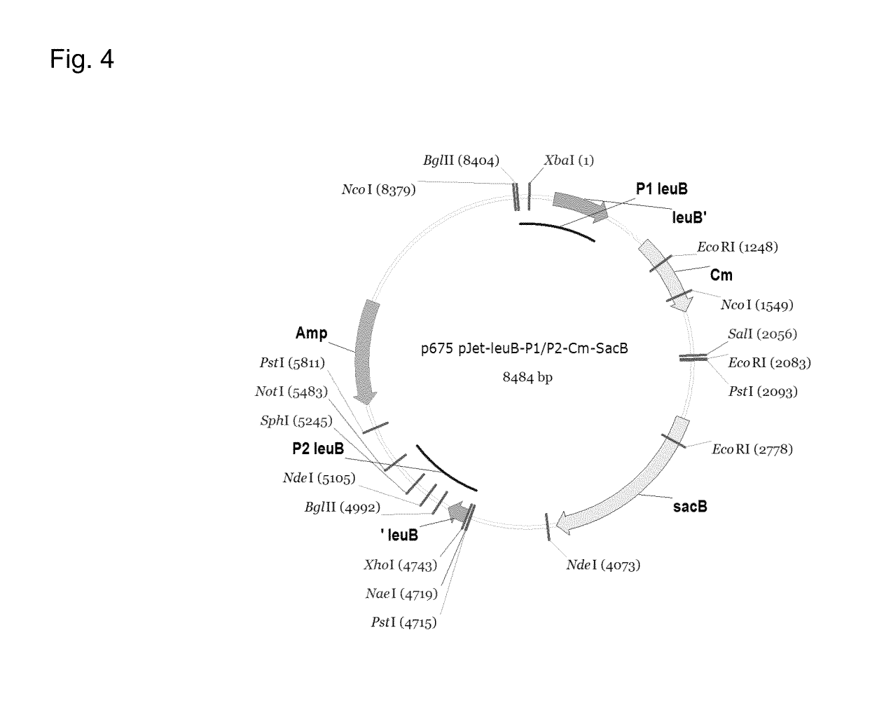

FIG. 4 shows the plasmid map of the plasmid #675, employed for the generation of a first gene inactivation in the leuB gene via the insertion of a first recombinant nucleic acid including a selectable marker (chloramphenicol=Cm) and a counterselectable marker (sacB). P1 leuB and P2 leuB denote the 5′- and 3′-platforms used for the insertion of the first nucleic acid into the leuB gene of the cyanobacterium via homologous recombination resulting in a truncated inactive variant of leuB.

SEQ ID NO. 4 shows the DNA sequence of the plasmid #675 indicating the start and end points of various genes encoded on this plasmid. This plasmid includes from nucleotides 205 to 670 ‘leuB, a 5’ part of truncated leuB coding sequence, from nucleotides 8415 to 670 “P1\leuB”, the 5′ recombination platform of leuB from Synechocystis PCC6803, from nucleotides 6009 . . . 6866 the Ampicillin resistance marker (Amp), from nucleotides 2579 to 4000 the gene sacB from Bacillus subtilis (coding for levansucrase) as counterselectable marker, from nucleotides 1034 to 1693 the Chloramphenicol acetyltransferase sequence(Cm), from nucleotides 4748 to 5480 “P2\leuB”, the 3′ recombination platform of leuB from Synechocystis PCC6803 and from nucleotides 4748 to 4912 leuB′, the 3′ part of truncated leuB coding sequence.

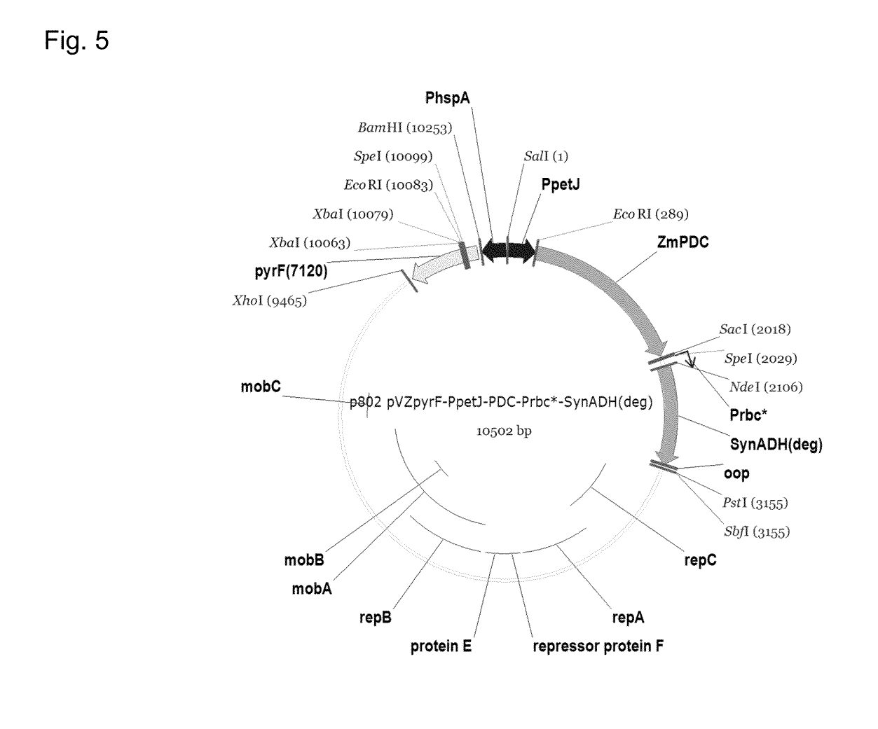

FIG. 5 shows the plasmid map of the plasmid #802, which can complement for a first gene inactivation in the pyrF gene and harbours the pyrF gene of Anabaena sp. PCC7120 under the control of PhspA in addition to the ethanologenic genes encoding ZmPdc controlled by PpetJ and SynAdh, which is under the transcriptional control of Prbc*.

SEQ ID NO. 5 depicts the DNA sequence of the plasmid #802 indicating the start and end points of various genes encoded on this plasmid. This plasmids includes from nucleotides 3118 to 3148 the terminator sequence oop, from nucleotides2107 to 3117 the gene coding for SynADH(deg), from nucleotides 2041 to 2105 Prbc*, the rbc promoter from Synechopcystis 6803-modified core promoter element (from −35 to ATG), from nucleotides 286 to 1995 the gene coding for ZmPDC, the pyruvate decarboxylase from Zymomonas mobilis, from nucleotides 7872 to 8153 the gene mobC, from nucleotides 5547 to 7673 the gene mobA, from nucleotides 6515 to 6925 the gene mob, from nucleotides 5544 to 6515 the gene repB, from nucleotides 5271 to 5483 the gene for protein\E, from nucleotides 5063 to 5269 the gene for repressor\protein\F, from nucleotides 4197 to 5033 the gene repA, from nucleotides 3356 to 4207 the gene repC, from nucleotides 9501 to 10217 the gene pyrF(7120), the pyrF gene from Anabaena PCC7120 for selection in E. coli KC8 and Syn6803 pyrF knockout, from nucleotides 10257 to 10501 PhspA, the hspA promoter from Synechocystis PCC6803, from nucleotides 1 to 285 PpetJ, the copper dependent promoter from Syn6803.

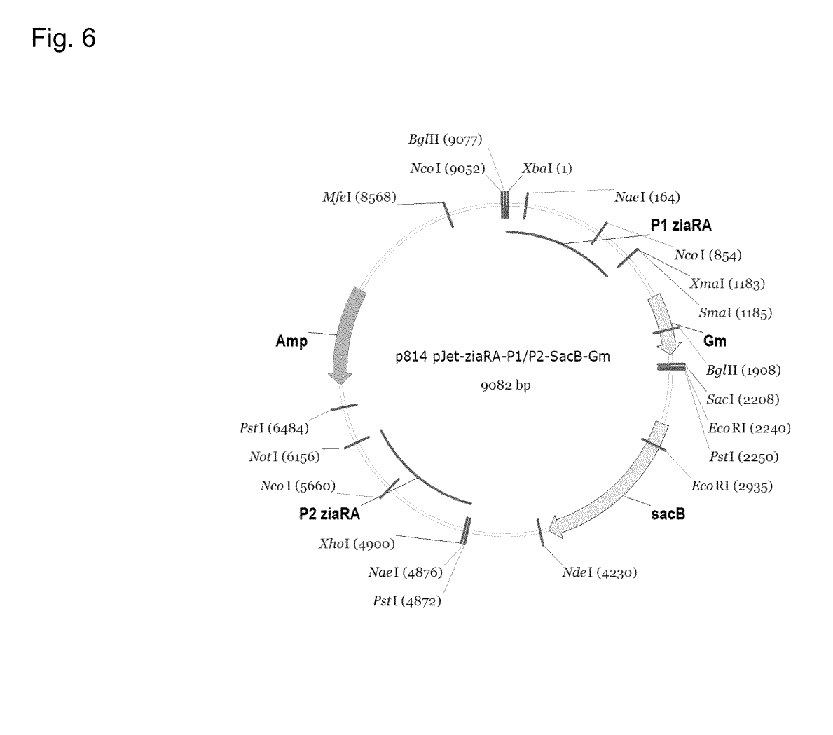

FIG. 6 shows the plasmid map of the plasmid #814, employed for the generation of a first gene inactivation in the ziaRA gene via the insertion of a first recombinant nucleic acid including a selectable marker (Gm) and a counterselectable marker (sacB). P1 ziaRA and P2 ziaRA denote the 5′- and 3′-platforms used for the insertion of the first nucleic acid into the ziaRA genes of the cyanobacterium via homologous recombination resulting in a truncated inactive variant of ziaRA.

SEQ ID NO. 6 shows the DNA sequence of the plasmid #814 indicating the start and end points of various genes encoded on this plasmid. This plasmid includes from nucleotides 1 to 1182 “P1\ziaRA”, the 5′ recombination platform of ziaRA from Synechocystis PCC6803, from nucleotides 1594 to 2127 the gentamicin-3-acetyltransferase (Gm), from nucleotides 6682 to 7539 the Ampicillin resistance gene (Amp), from nucleotides 2736 to 4157 the sacB gene, from nucleotides 4900 to 6155 “P2\ziaRA”, the 3′ recombination platform of ziaRA from Synechocystis PCC6803.

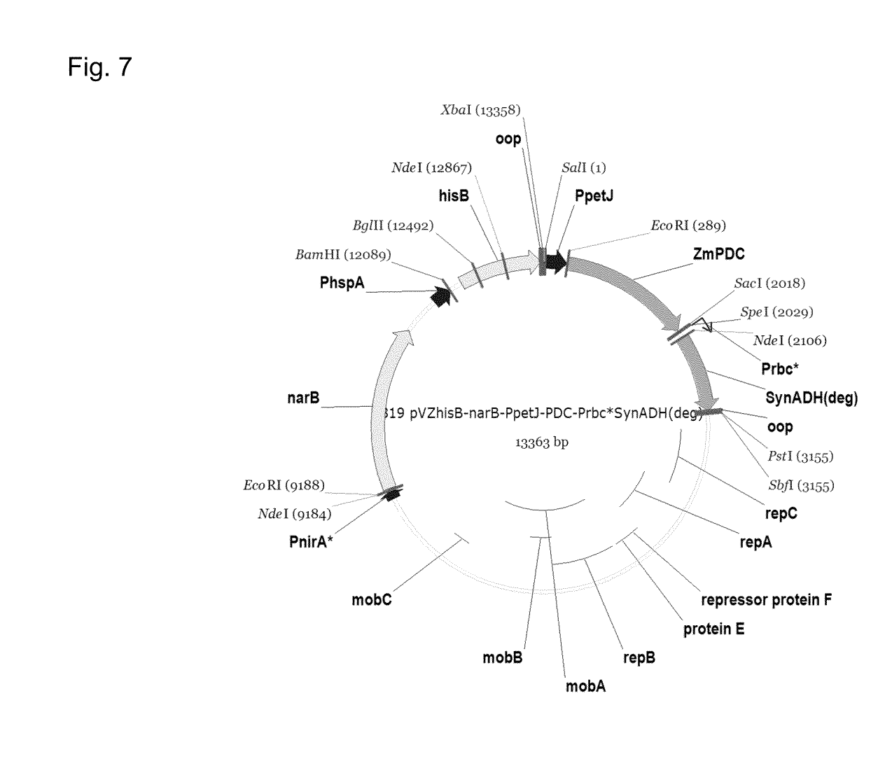

FIG. 7 shows the plasmid map of the plasmid #819, which can complement for a first gene inactivation in the narB gene and harbours the narB gene under the control of PnirA* along with the hisB gene for selection in E. coli KC8 strain (histidine auxotroph) in addition to the ethanologenic genes encoding ZmPdc controlled by PpetJ and SynAdh, which is under the transcriptional control of Prbc*.

SEQ ID NO. 7 depicts the DNA sequence of the plasmid #819 indicating the start and end points of various genes encoded on this plasmid. This plasmid includes from nucleotides 286 to 1995 the gene coding for ZmPDC, from nucleotides 2041 to 2105 Prbc*, from nucleotides 2107 to 3117 the gene coding for SynADH(deg), from nucleotides 3118 to 3148 the terminator sequence oop, from nucleotides 1 to 285 PpetJ, from nucleotides 9065 to 9184 PnirA*, the truncated nirA promoter from Synechocystis PCC6803, from nucleotides 9208 to 11349 the gene narB, from nucleotides 11839 to 12088 PhspA, from nucleotides 3356. to 4207 the gene repC, from nucleotides 4197 to 5033 the gene repA, from nucleotides 5063 to 5269 the gene for repressor\protein\F, from nucleotides 5271 to 5483 the gene for protein\E, from nucleotides 5544 to 6515 the gene repB, from nucleotides 6515 to 6925 the gene mob, from nucleotides 5547 to 7673 the gene mobA, from nucleotides 7872 to 8153 the gene mobC, from nucleotides 13327 to 13357 oop, and from nucleotides 12259 to 13326 the gene for hisB from E.coli K-12 for selection in E.coli KC8.

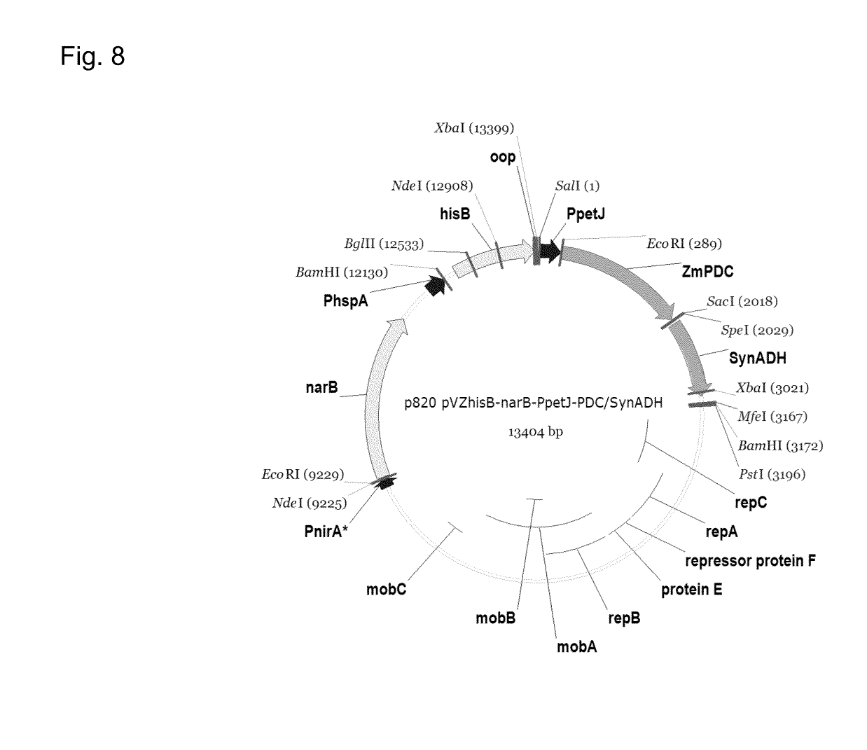

FIG. 8 shows the plasmid map of the plasmid #820, which can complement for a first gene inactivation in the narB gene and harbours the narB gene under the control of PnirA* along with the hisB gene for selection in E. coli KC8 strain (histidine auxotroph) in addition to the ethanologenic genes encoding ZmPdc and SynAdh, which are both under the transcriptional control of PpetJ.

SEQ ID NO. 8 depicts the DNA sequence of the plasmid #820 indicating the start and end points of various genes encoded on this plasmid. This plasmids harbours from nucleotides 12300 to 13367 the hisB gene from E.coli K-12 for selection in E.coli KC8, from nucleotides 13368 to 13398 the oop sequence, from nucleotides 7913 to 8194 the gene mobC, from nucleotides 5588 to 7714 the gene mobA, from nucleotides 6556 to 6966 the gene mob, from nucleotides 5585 to 6556 the gene repB, from nucleotides 5312 to 5524 the gene for protein\E, from nucleotides 5104 to 5310 the gene for repressor\protein\F, from nucleotides 4238 to 5074 the gene repA, from nucleotides 3397 to 4248 the gene repC, from nucleotides 11880 to 12129 PhspA, from nucleotides 9249 to 11390 the narB gene from Synechocystis PCC6803 for selection in Syn6803 narB-knockout, from nucleotides 9106 to 9225 PnirA*, from nucleotides 286 to 1995 the gene for ZmPDC, from nucleotides 1 to 285 PpetJ, and from nucleotides 2064 to 3074 the gene coding for SynADH.

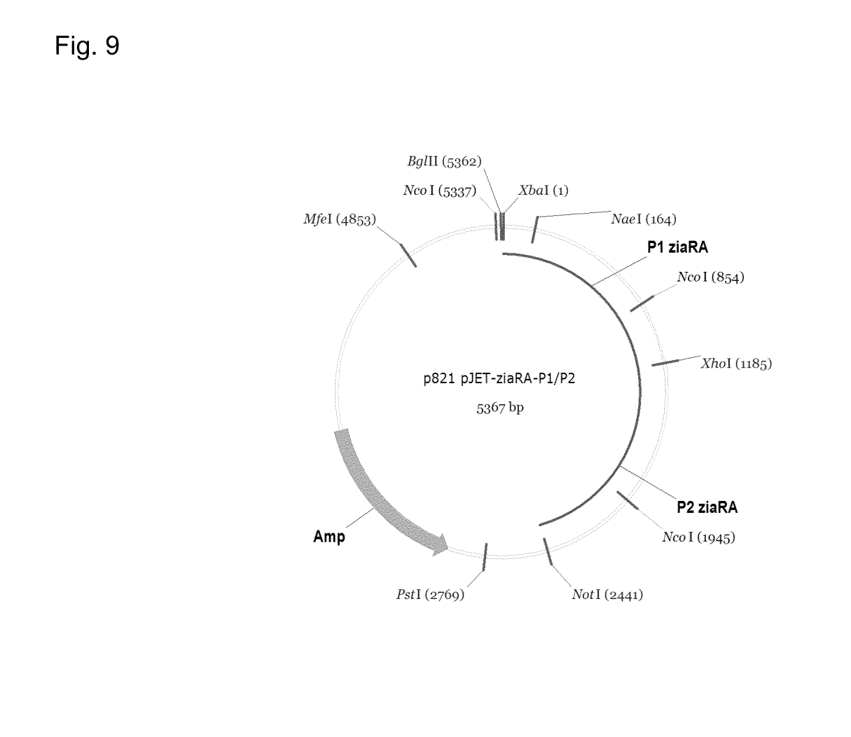

FIG. 9 shows the plasmid map of the plasmid #821, employed for the replacement of the first recombinant nucleic acid inserted into the ziaRA gene via insertion of a second recombinant nucleic acid lacking both the selectable marker and the counterselectable marker. P1 ziaRA and P2 ziaRA denote the 5′- and 3′-platforms used for the insertion of the second nucleic acid into the ΔziaRA gene formed upon insertion of the first nucleic acid via homologous recombination.

SEQ ID NO. 9 depicts the DNA sequence of the plasmid #821 indicating the start and end points of various genes encoded on this plasmid. This plasmid includes from nucleotides 1185 to 2440 “P2\ziaRA”, the 3′ recombination platform of ziaRA from Synechocystis PCC6803, from nucleotides 2967 to 3824 the Ampicillin resistance gene, from nucleotides 1 to 1182 “P1\ziaRA”, the 5′ recombination platform of ziaRA from Synechocystis PCC6803.

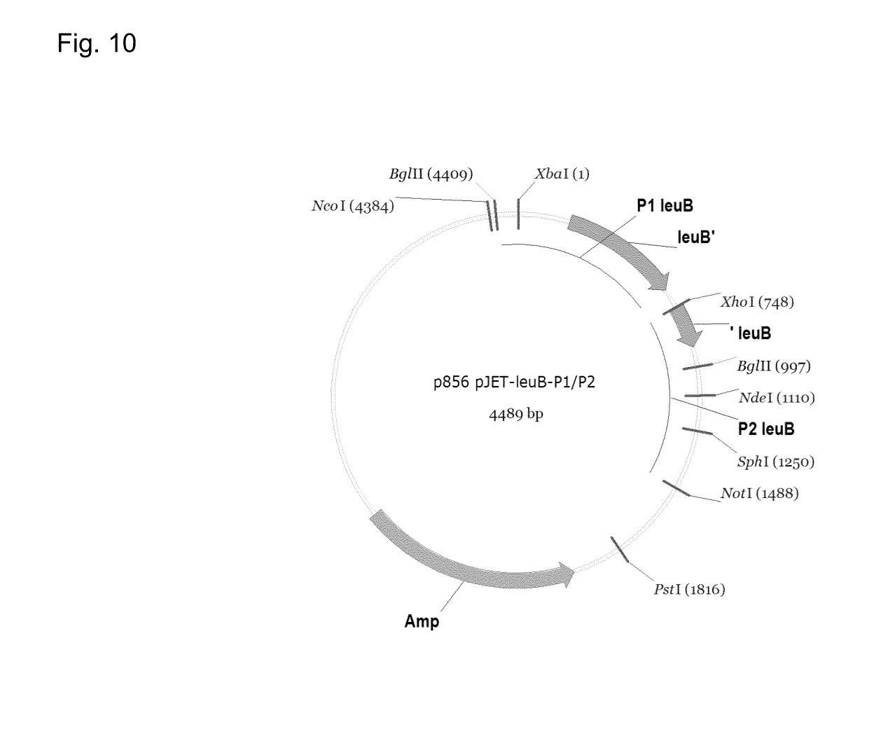

FIG. 10 shows the plasmid map of the plasmid #856, employed for the replacement of the first recombinant nucleic acid inserted into the leuB gene via insertion of a second recombinant nucleic acid lacking both the selectable marker and the counterselectable marker. P1 leuB and P2 LeuB denote the 5′- and 3′-platforms used for the insertion of the second nucleic acid into the ΔleuB gene formed upon insertion of the first nucleic acid via homologous recombination.

SEQ ID NO. 10 depicts the DNA sequence of the plasmid #856 indicating the start and end points of various genes encoded on this plasmid. This plasmids contains from nucleotides 753 to 917 the gene ‘leuB, the 3’ part of truncated leuB coding sequence, from nucleotides 753 to 1485 “P2\leuB”, the 3′ recombination platform of leuB from Synechocystis PCC6803, from nucleotides 2014 to 2871 the Ampicillin resistance cassette, from nucleotides 4420 to 670 “P1\leuB”, the 5′ recombination platform of leuB from Synechocystis PCC6803, from nucleotides 205 to 670 leuB′, the 5′ part of truncated leuB coding sequence.

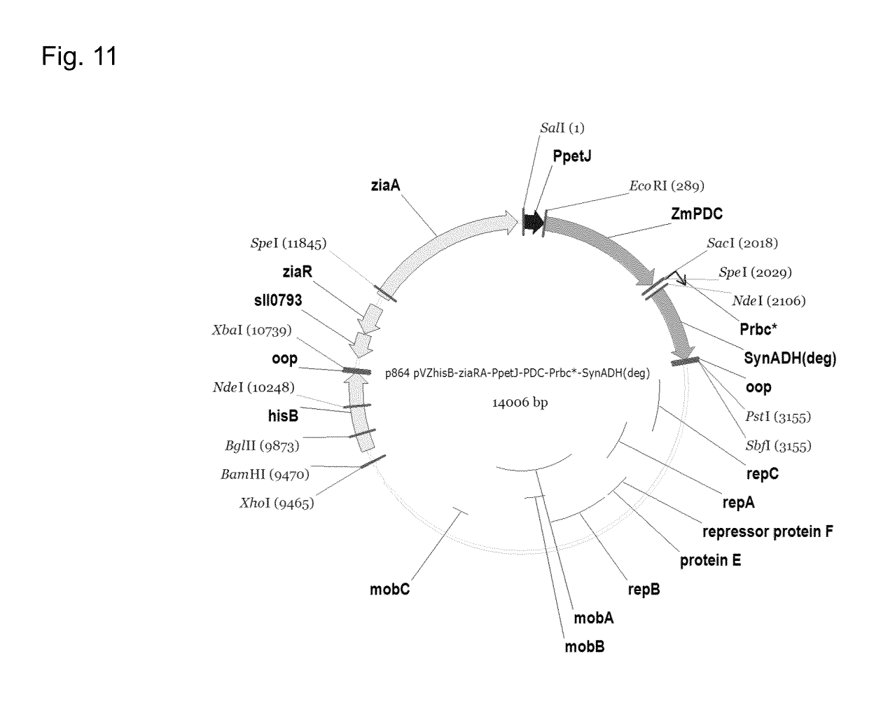

FIG. 11 shows the plasmid map of the plasmid #864, which can complement for a first gene inactivation in the ziaRA genes and harbours the ziaA and the ziaR gene along with the hisB gene for selection in E. coli KC8 strain (histidine auxotroph) in addition to the ethanologenic genes encoding ZmPdc controlled by PpetJ and SynAdh_{deg}, which is under the transcriptional control of Prbc*.

SEQ ID NO. 11 depicts the DNA sequence of the plasmid #864 indicating the start and end points of various genes encoded on this plasmid. This plasmid contains from nucleotides 9640 to 10707 the hisB gene from E. coli K-12 for selection in E.coli KC8, from nucleotides 10708 to 10738 the oop terminator sequence, from nucleotides 11787 to 13952 the gene ziaA, from nucleotides 11237 to 11635 the ziaR gene, from nucleotides 10899 to 11225 sll0793 from Synechocystis PCC6803-gene of unknown function, from nucleotides 1 to 285 PpetJ, from nucleotides 3356 to 4207 the gene repC, from nucleotides 4197 to 5033 the gene repA, from nucleotides 5063 to 5269 the gene for the repressor\protein\F, from nucleotides 5271 to 5483 the gene for protein\E, from nucleotides 5544 to 6515 the gene repB, from nucleotides 6515 to 6925 the gene mob, from nucleotides 5547 to 7673 the gene mobA, from nucleotides 7872 to 8153 the gene mobC, from nucleotides 286 to 1995 the gene coding for ZmPDC, from nucleotides 2041 to 2105 Prbc*, from nucleotides 2107 to 3117 the gene coding for SynADH(deg), and from nucleotides 3118 to 3148 the oop terminator.

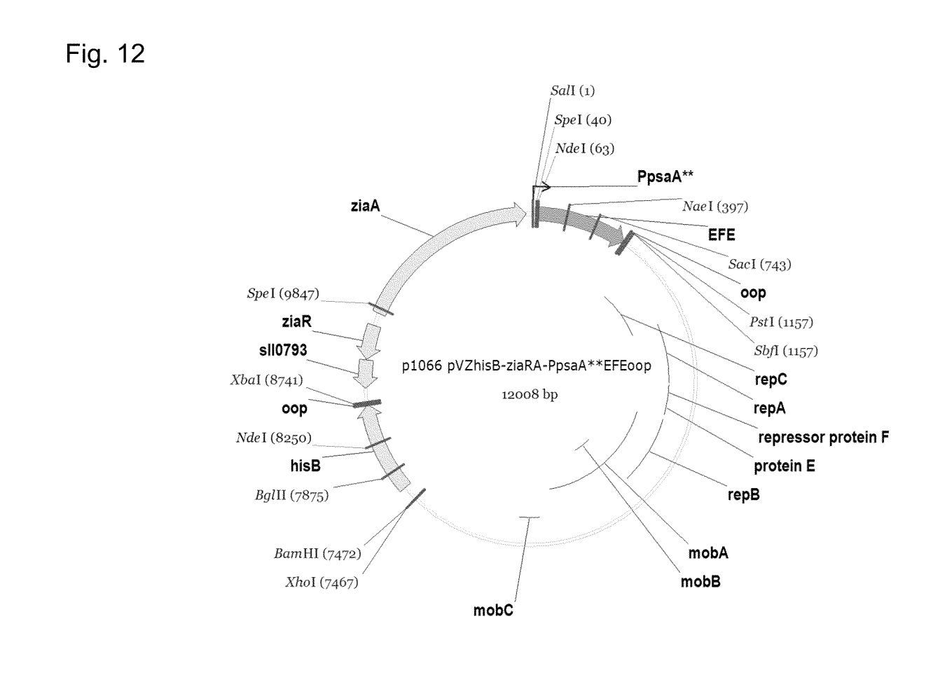

FIG. 12 shows the plasmid map of the plasmid #1066, which can complement for a first gene inactivation in the ziaRA genes and harbours the ziaA and the ziaR gene along with the hisB gene for selection in E. coli KC8 strain (histidine auxotroph) in addition to the efe gene coding for the ethylene forming enzyme under the transcriptional control of PpsaA*.

SEQ ID NO. 12 depicts the DNA sequence of the plasmid #1066 indicating the start and end points of various genes encoded on this plasmid. This plasmid contains from nucleotides 6 to 60 PpsaA**, the psaA promoter from Synechocystis PCC6803-modified core promoter element (from −35 to ATG), from nucleotides 64 to 1116 the gene EFE, coding for the ethylene forming enzyme gene from Pseudomonas syringae pv. phaseolicola-codon optimized, from nucleotides 1120 to 1151 the oop terminator, from nucleotides 5874 to 6155 the gene mobC, from nucleotides 3549 to 5675 the gene mobA, from nucleotides 4517 to 4927 the gene mob, from nucleotides 3546 to 4517 the gene repB, from nucleotides 3273 to 3485 the gene for protein\E, from 3065 to 3271 the gene for repressor\protein\F, from nucleotides 2199 to 3035 the gene repA, from nucleotides 1358 to 2209 the gene repC, from nucleotides 8901 to 9227 sll0793 from Synechocystis PCC6803, from nucleotides 9239 to 9637 the gene ziaR, from nucleotides 9789 to 11954 the gene ziaA, from nucleotides 8710 to 8740 the oop terminator, from nucleotides 7642 to 8709 the gene hisB.

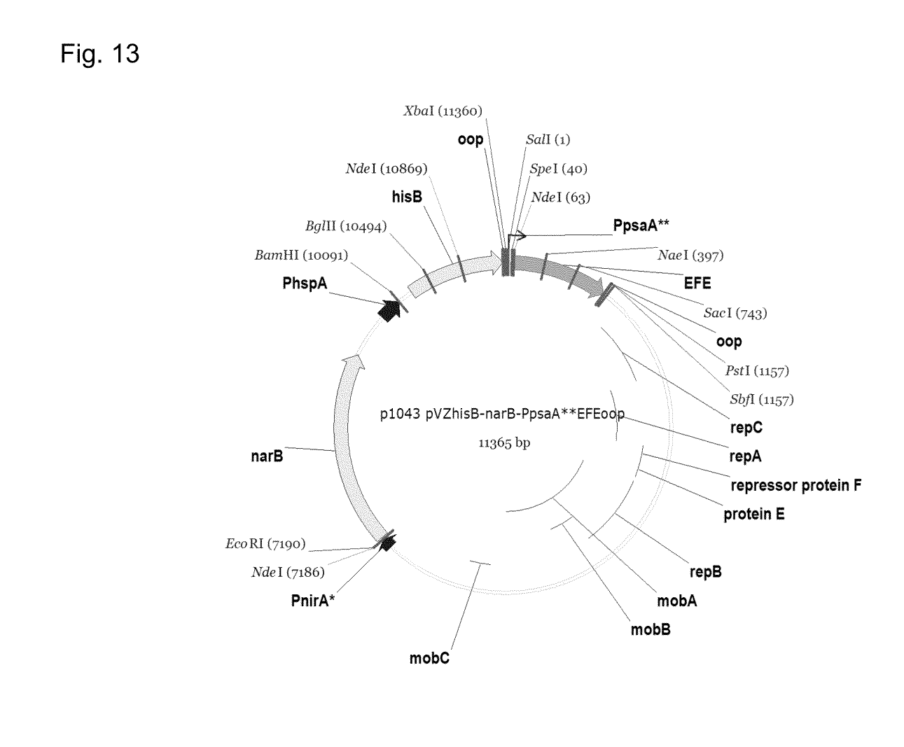

FIG. 13 shows the plasmid map of the plasmid #1043, which can complement for a first gene inactivation in the narB gene and harbours the narB gene under the control of PnirA* along with the hisB gene for selection in E. coli KC8 strain (histidine auxotroph) in addition to the efe gene coding for the ethylene forming enzyme under the transcriptional control of PpsaA*.

SEQ ID NO. 13 depicts the DNA sequence of the plasmid #1043 indicating the start and end points of various genes encoded on this plasmid. This plasmid includes from nucleotides 1120 to 1151 the oop terminator, from nucleotides 64 to 1116 the gene EFE, from nucleotides 6 to 60 PpsaA**, the psaA promoter from Synechocystis PCC6803-modified core promoter element (from −35 to ATG), from nucleotides 7067 to 7186 PnirA*, the truncated nirA promoter from Synechocystis PCC6803, from nucleotides 7210 to 9351 the gene narB, from 9841 to 10090 PhspA, from nucleotides 1358 to 2209 the gene repC, from nucleotides 2199 to 3035 the repA, from nucleotides 3065 to 3271 the gene for repressor\protein\F, from nucleotides 3273 to 3485 the gene for protein\E, from nucleotides 3546 to 4517 the gene repB, from nucleotides 4517 to 4927 the gene mob, from nucleotides 3549 to 5675 the gene mobA, from nucleotides 5874 to 6155 the gene mobC, from nucleotides 11329 to 11359 the oop terminator, and from nucleotides 10261 to 11328 the gene hisB.

FIG. 14A shows a schematic drawing of the general strategy for creating a first gene inactivation in the pryF gene and insertion of a first production gene for the first chemical compound ethanol directly into the genome of the cyanobacterium as a comparative example;

FIG. 14B shows a DNA agarose gel of genetically enhanced cyanobacteria obtained via the gene inactivation strategy depicted in FIG. 14A, evidencing that complete segregation of the genetically enhanced cyanobacteria could not be obtained by using this gene inactivation strategy in spite of uracil addition and strong selection pressure by high concentrations of kanamycin.

FIG. 14C shows an agarose DNA gel evidencing that via the use of a first extrachromosomal plasmid for the first gene inactivation strategy of pyrF a complete segregation of the genetically enhanced cyanobacteria can be obtained.

FIG. 14D shows an agarose DNA gel evidencing that after selection on sucrose via the use of the second recombinant nucleic acid ΔpyrF the first recombinant nucleic acid ΔpyrF(SacB/Cm) is replaced by subjecting the cells to the second counterselectable marker.

FIG. 14E shows the ethanol production rate of genetically enhanced cyanobacteria harbouring a first gene inactivation in the pyrF gene and also including a first extrachromosomal plasmid #802 with the pyrF gene and additional first production genes encoding Adh and Pdc enzymes (denoted with #802) in comparison to the ABR gene containing controls including the plasmid #550 (denoted with #550).

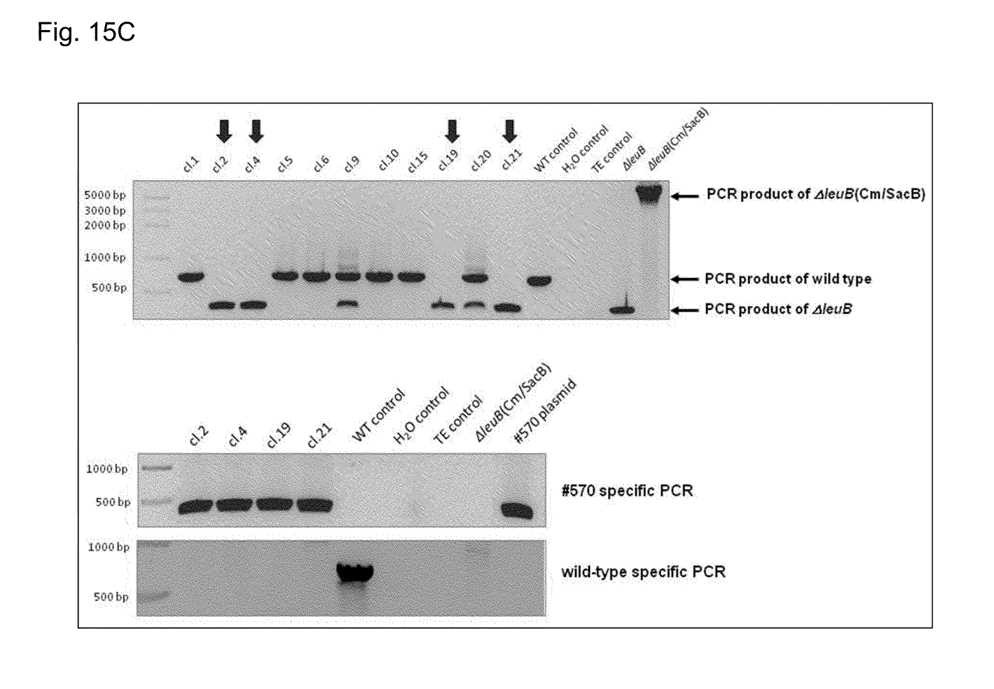

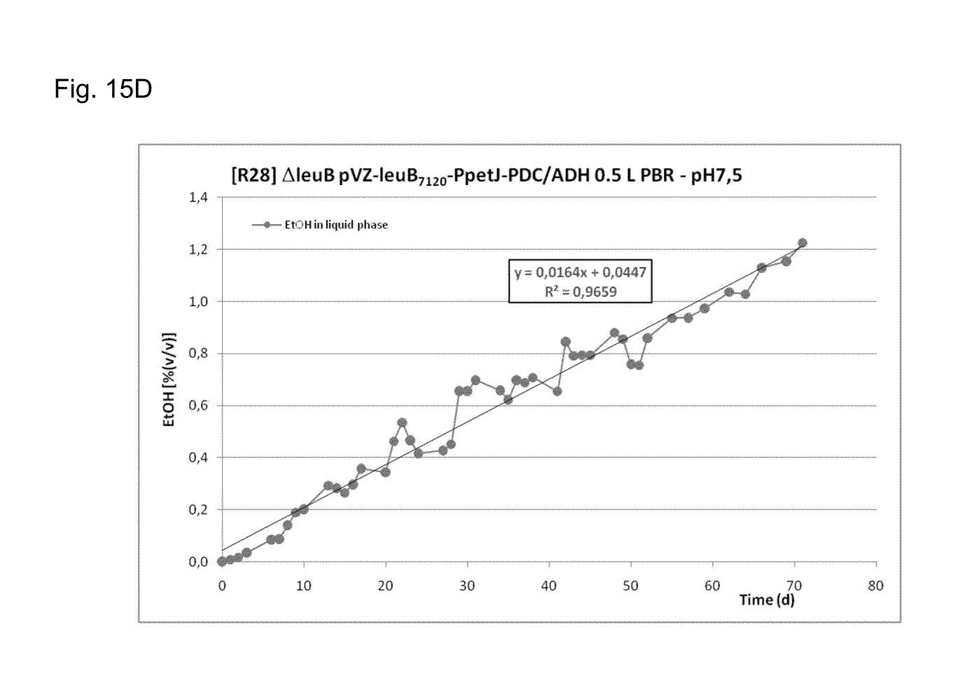

FIG. 15A schematically depicts a successful gene inactivation strategy for obtaining a first gene inactivation in the leuB gene of Synechocystis sp. PCC 6803.

FIG. 15B shows a DNA agarose gel of completely segregated genetically enhanced cyanobacteria harbouring a first gene inactivation in the leuB gene obtained after step 2) of the successful complementation strategy shown in FIG. 15A;

FIG. 15C shows an agarose DNA gel evidencing that after selection on sucrose after the transformation with the second recombinant nucleic acid ΔleuB the first recombinant nucleic acid ΔleuB(SacB/Cm) is replaced by subjecting the cells to the second counterselectable marker.

FIG. 15D is a graph depicting the long-term ethanol production rate for a culturing time of over 70 days for genetically enhanced cyanobacteria harbouring a first gene inactivation in the leuB gene and in addition also including a first extrachromosomal plasmid with the leuB gene and additional first production genes encoding Adh and Pdc enzymes for ethanol production.

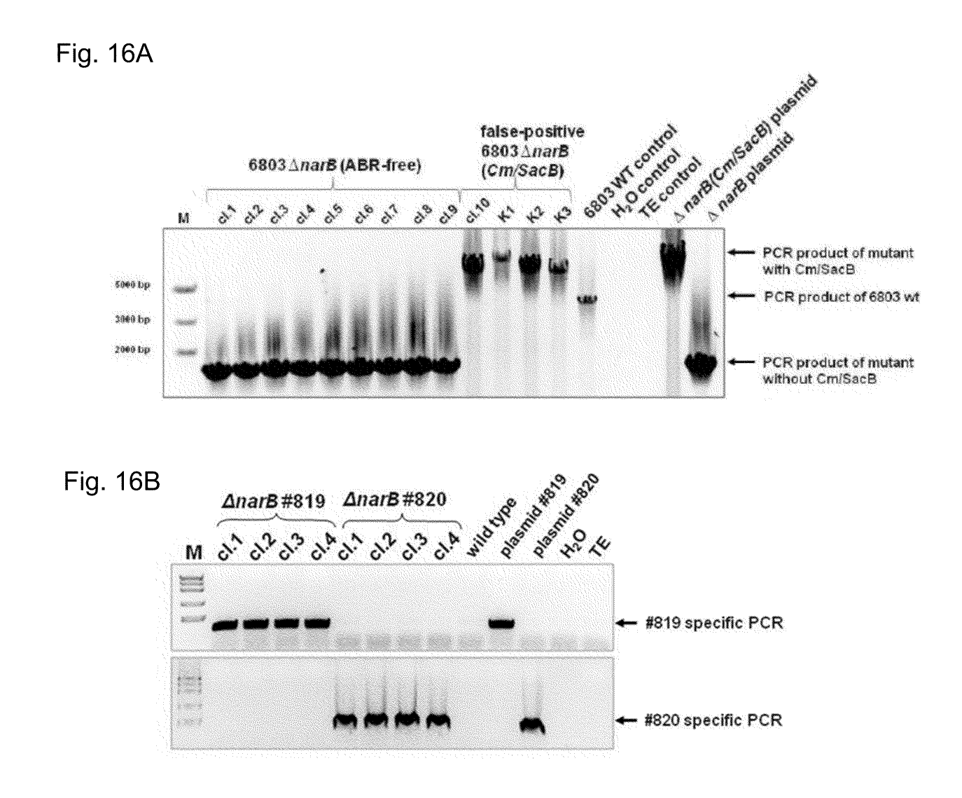

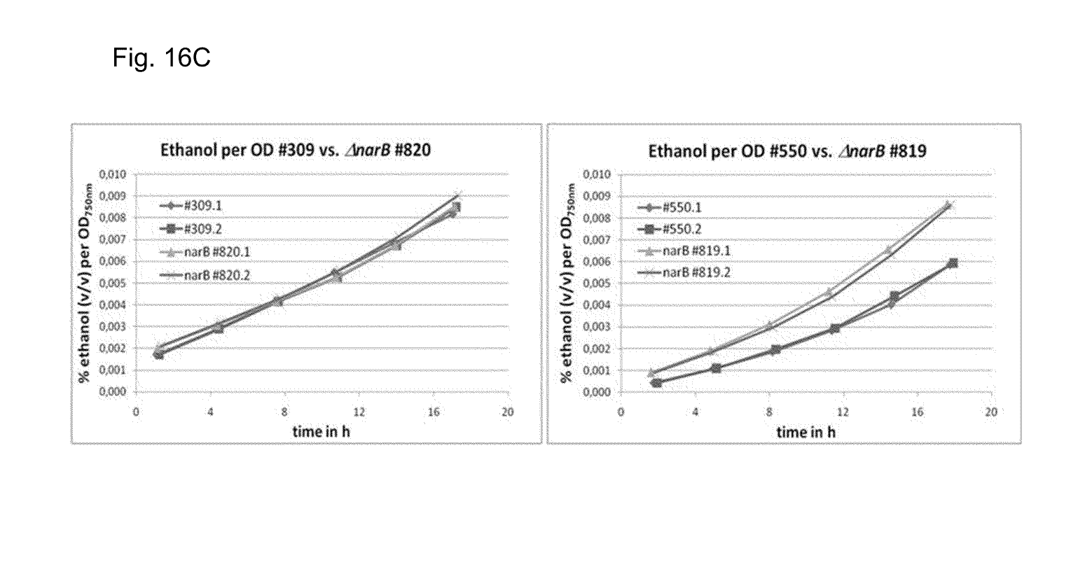

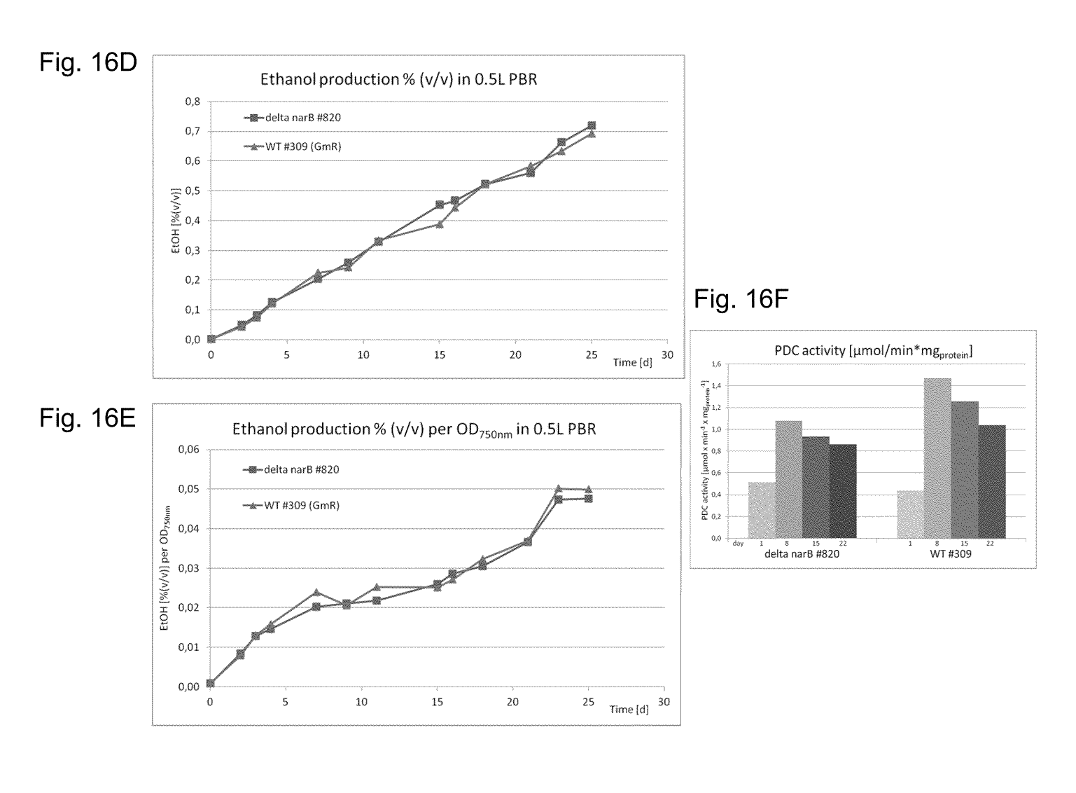

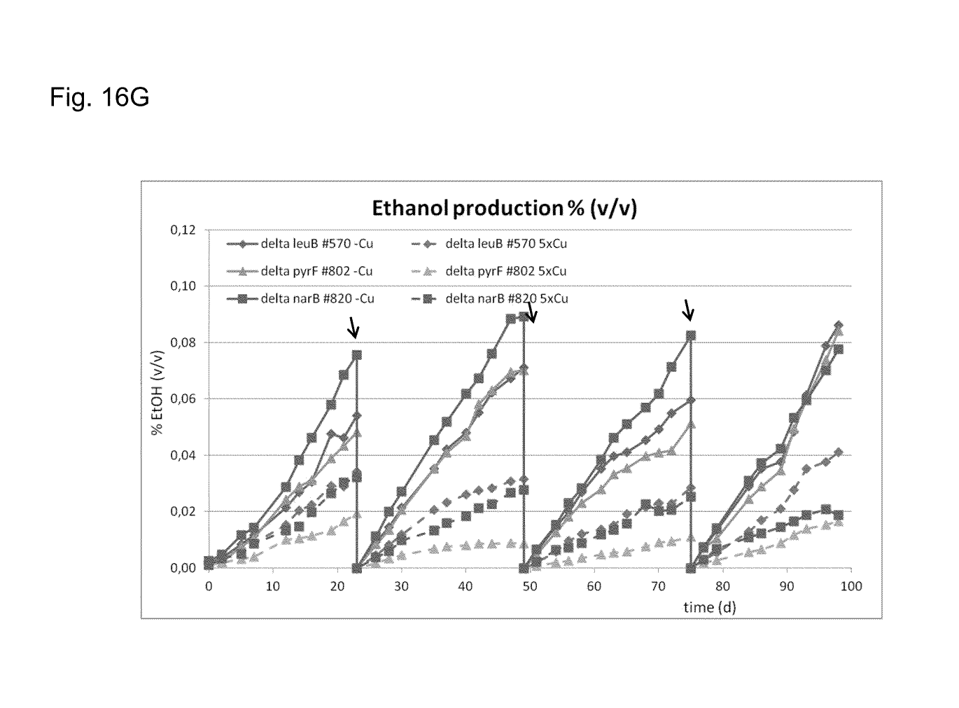

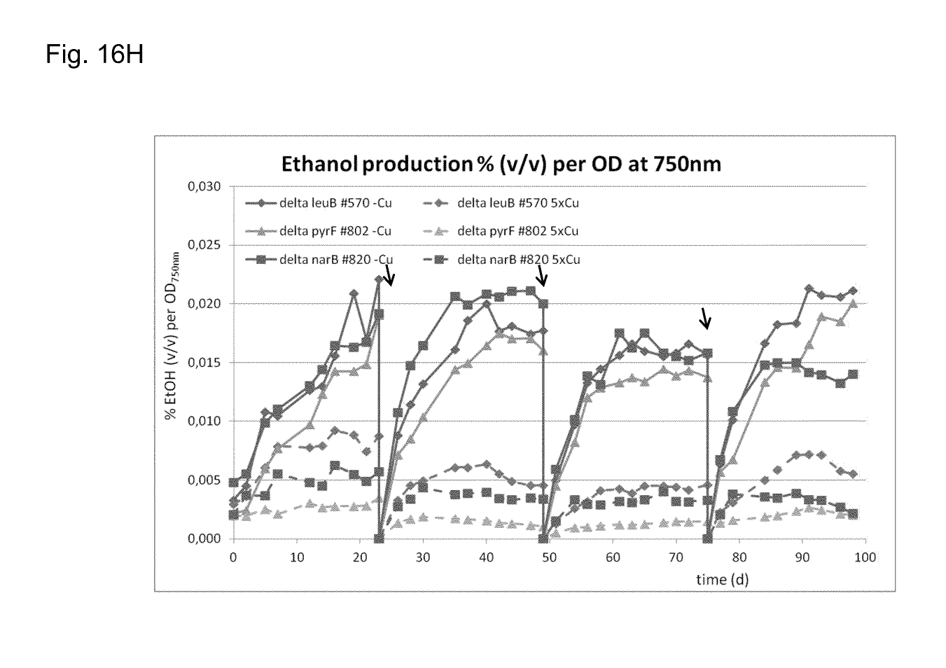

FIG. 16A shows a DNA agarose gel of completely segregated genetically enhanced cyanobacteria having a first gene inactivation in the narB gene leading to an ammonia dependent phenotype (product of method step i4).

FIG. 16B shows an agarose gel of successful complemented ΔnarB colonies by conjugation with ethanologenic pVZ-narB plasmids #819 and #820 as first extrachromosomal plasmids (product of method step ii).

FIG. 16C shows the ethanol production rate of genetically enhanced cyanobacteria comprising a first gene inactivation in the narB gene and in addition also including a first extrachromosomal plasmid including a gene complementing for the first gene inactivation in the narB gene and also first production genes encoding for Adh and Pdc enzymes for ethanol production (plasmids #820 and #819, resp.) in comparison to the ABR gene containing controls (#309 and #550).

FIG. 16D shows ethanol production and FIG. 16E shows ethanol production normalized on OD at 750 nm of a Synechocystis ΔnarB strain complemented with the narB-ethanologenic plasmid #820 in comparison to a wild type with conventional ethanologenic plasmid “WT #309”.

FIG. 16F is a bar graph showing the measured PDC activities at four different time points within the cultivation experiment.

FIG. 16G shows ethanol production and FIG. 16H shows ethanol production per OD of the ABR free strains ΔleuB #570 (pVZleuB-PpetJ-PDC/synADH), ΔpyrF #802 (pVZpyrF-PpetJ-PDC-Prbc*-synADH_{deg}) and ΔnarB #820 (pVZhisB-PnirA*-narB-PpetJ-PDC/synADH).

FIG. 16I is a bar graph showing the PDC activity in samples taken after 84 days of cultivation.

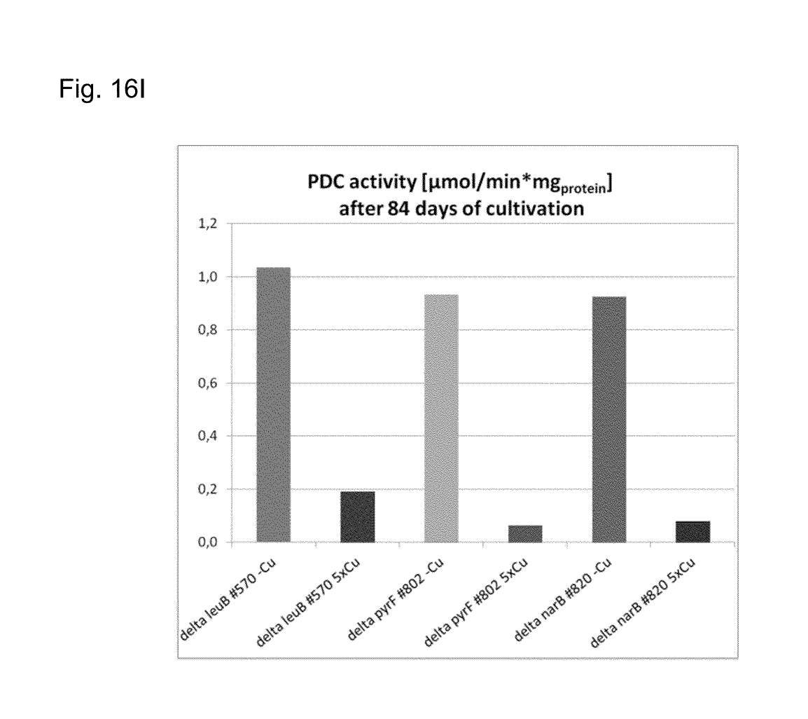

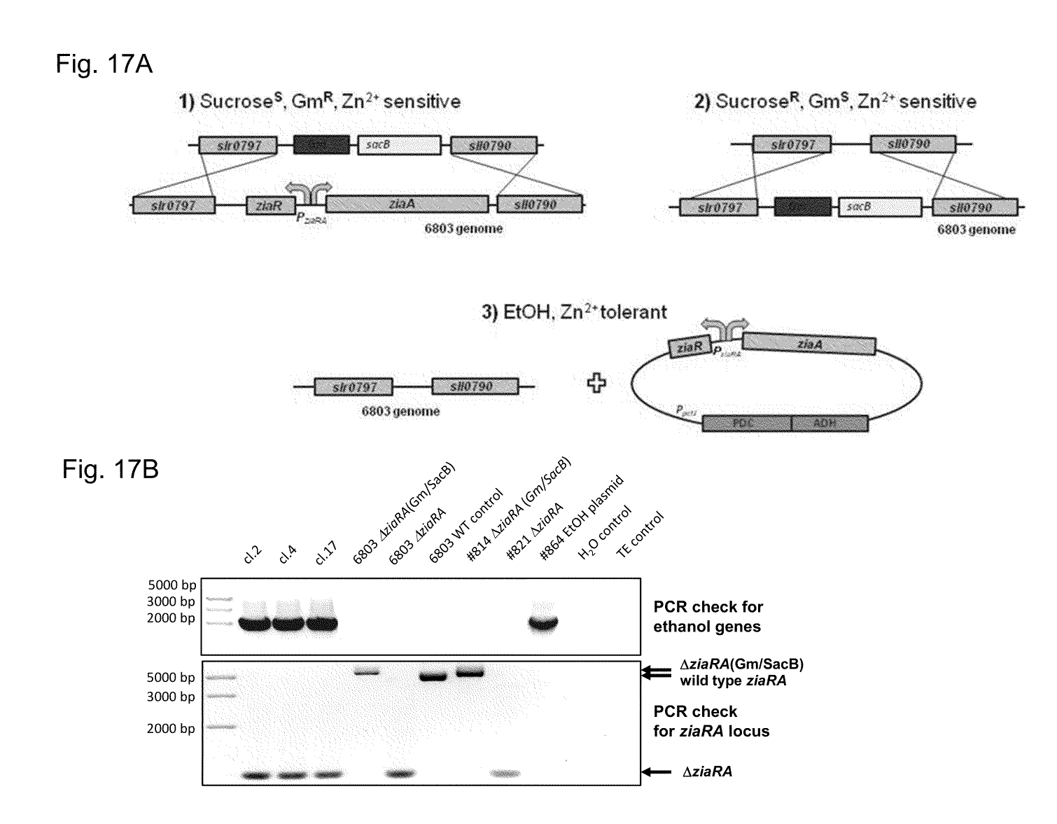

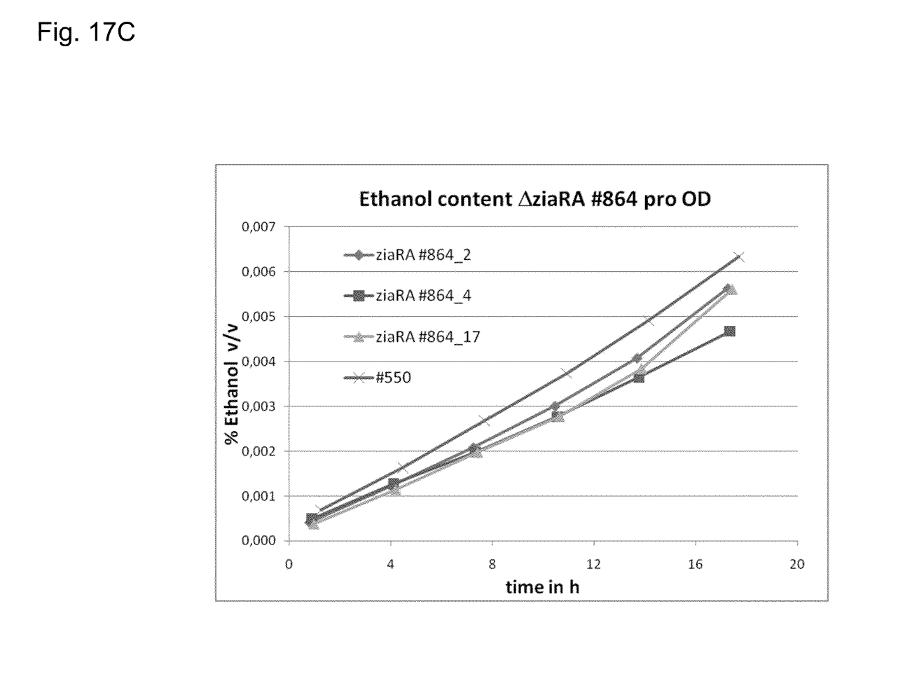

FIG. 17A shows schematically the gene inactivation strategy for creating a first gene inactivation in the ziaRA genes, also involving a first extrachromosomal plasmid comprising the ziaRA genes along with the first production genes encoding for Adh and Pdc enzymes for ethanol production.

FIG. 17B again shows a DNA agarose gel evidencing that complete first gene inactivation in the ziaRA gene could be achieved also in cyanobacteria also including a first extrachromosomal plasmid #864 with the ziaRA genes (for restoration of the zinc tolerance) and an additional first production genes encoding Adh and Pdc enzymes for ethanol production.

FIG. 17C depicts the ethanol production rate of genetically enhanced cyanobacteria harbouring the first gene inactivation of the ziaRA genes and also including a first extrachromosomal plasmid including first production genes encoding Pdc and Adh enzymes(#864) in comparison to the ABR gene containing control (#550).

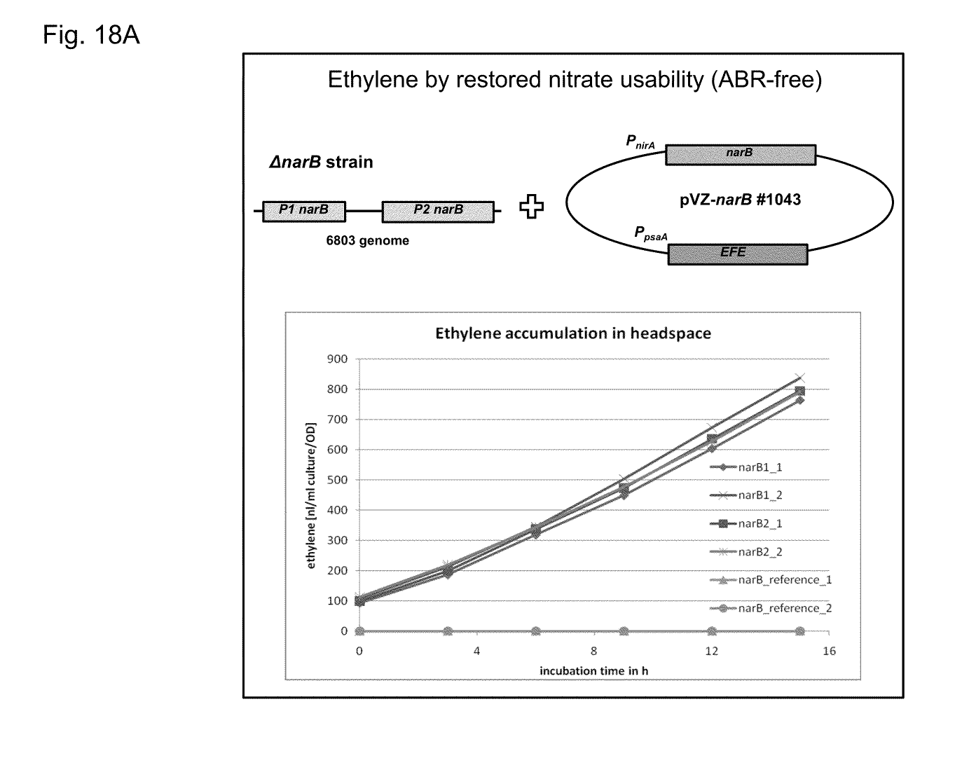

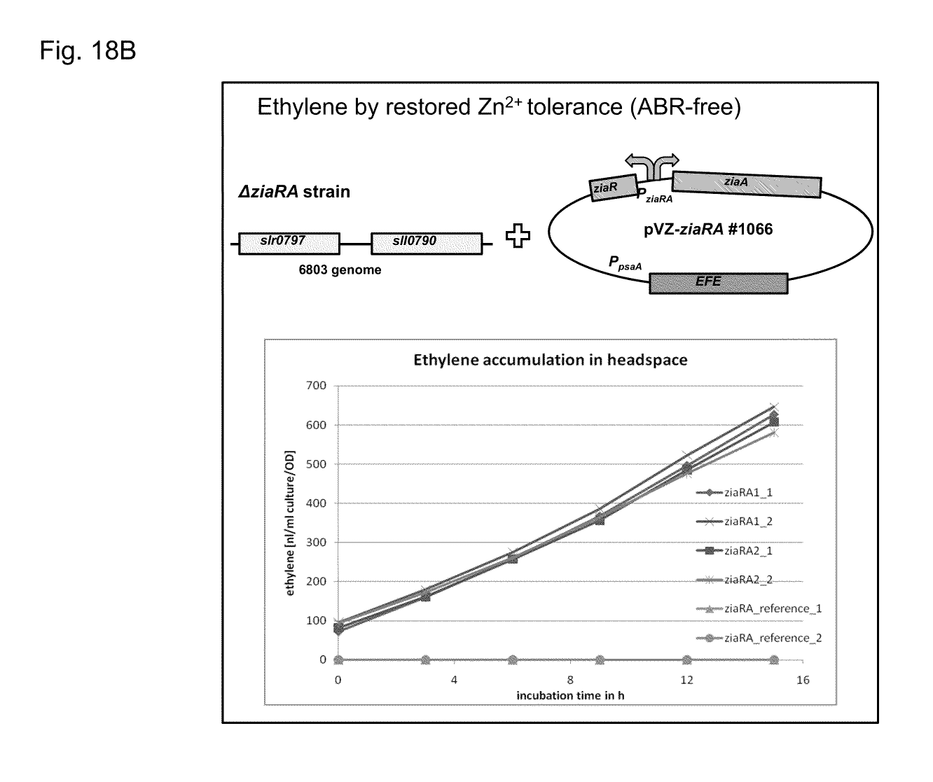

FIG. 18A shows the general features of a genetically enhanced cyanobacterium harbouring a genomic first gene inactivation in the narB gene and also including a first extrachromosomal plasmid including a narB gene and a gene encoding an ethylene forming enzyme; The below graph shows the ethylene production rate of these genetically enhanced cyanobacteria.

FIG. 18B shows the general features of a genetically enhanced cyanobacterium harbouring a genomic first gene inactivation in the ziaRA genes and also including a first extrachromosomal plasmid including the ziaRA genes and a gene encoding an ethylene forming enzyme; The below graph shows the ethylene production rate of these genetically enhanced cyanobacteria.

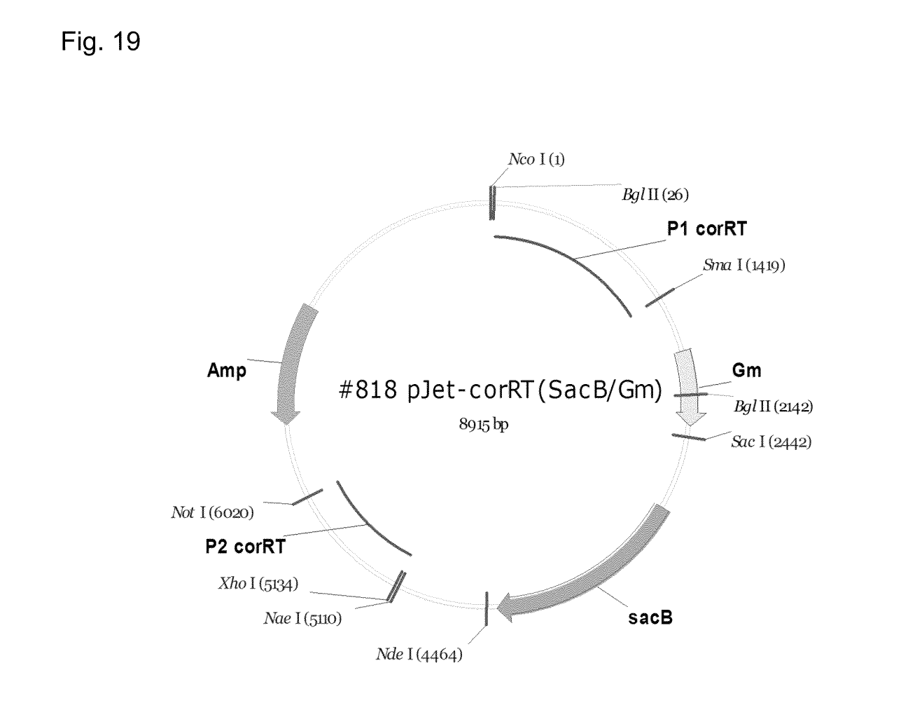

FIG. 19 shows the plasmid map of the plasmid #818 employed to replace the endogenous corT gene in Synechocystis with a SacB/Gm cassette.

SEQ ID NO. 14 shows the DNA sequence of the plasmid #818 indicating the start and end points of various genes encoded on this plasmid. This plasmid contains from nucleotides 42 to 1418 “P1\corRT”, the platform for homologous recombination, from nucleotides 2970 to 4391 the gene sacB, from nucleotides 6546 to 7403 “Amp”, the ampicillin resistance gene, from 1828 to 2361 “Gm”, the gentamycin resistance gen, and from nucleotides 5136 to 6021 “P2\corRT”, the platform for homologous recombination.

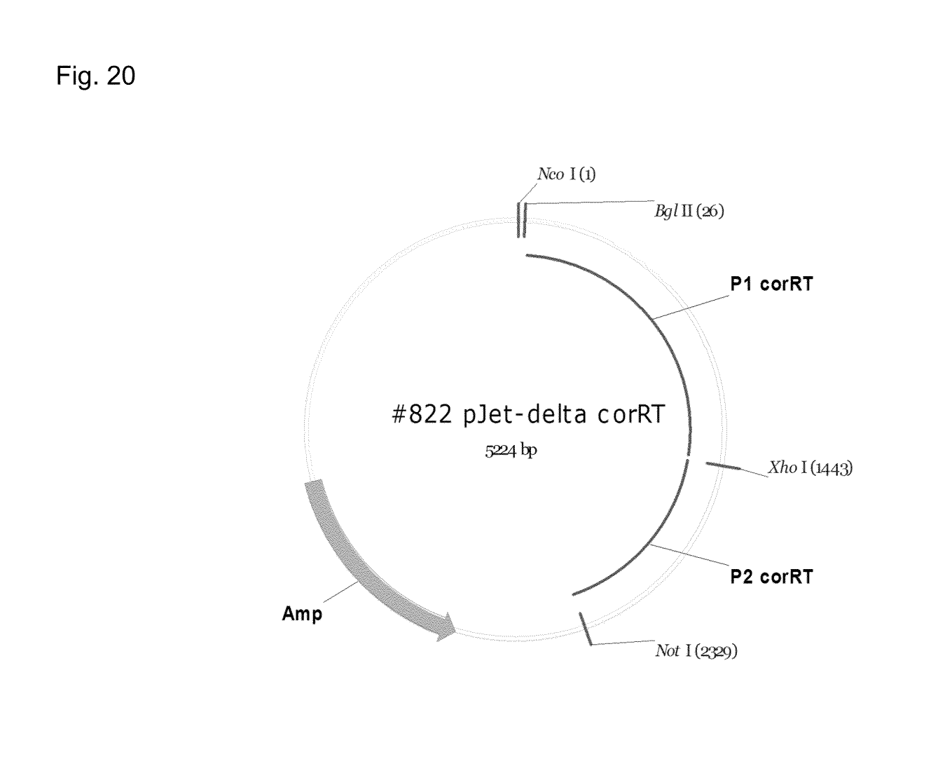

FIG. 20 shows the plasmid map of the plasmid #822, which is used in order to replace the sacB/Gm cassette, introduced via the plasmid #818 into Synechocystis with a AcorT sequence.

SEQ ID NO. 15 shows the plasmid map of plasmid #822. This plasmid contains from nucleotides 1445 to 2330 “P2\corRT” the platform for homologous recombination, from nucleotides 2855 to 3712 “Amp”, the Ampicillin resistance gen, from nucleotides 42 to 1418 the platform “P1\corRT”.

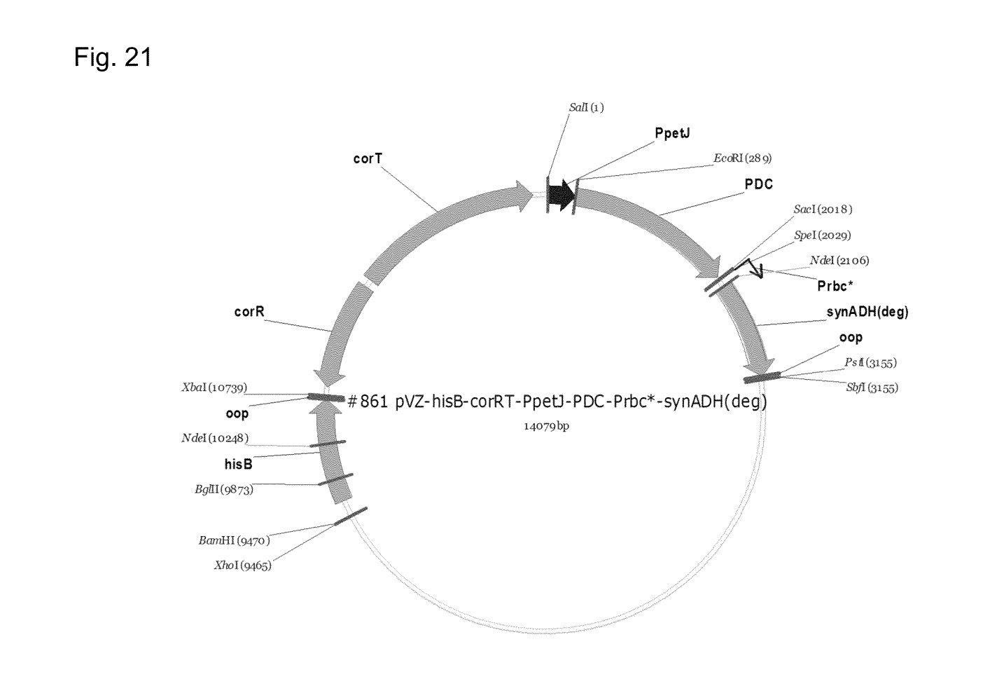

FIG. 21 shows the plasmid map of the first extrachromosomal plasmid #861 including the sequence pVZhisB-corRT-PpetJ-PDC-Prbc*-synADH_{deg} for ethanol production used for transformation of corRT gene inactivated cyanobacterial cells.

SEQ ID NO. 16 shows the DNA sequence of the plasmid #861 indicating the start and end points of various genes encoded on this plasmid. This plasmid includes from nucleotides 3118 to 3148 the oop terminator, from nucleotides 2107 to 3117 the gene coding for synADH(deg), from nucleotides 2041 to 2105 Prbc*, from nucleotides 1 to 288 PpetJ, from nucleotides 293 to 1993 the gene coding for PDC enzyme, from nucleotides 12022 to 13950 the gene coding for corT, from nucleotides 10828 to 11940 the gene coding for corR, from nucleotides 10708 to 10738 the oop terminator, from nucleotides 9640 to 10707 the gene hisB.

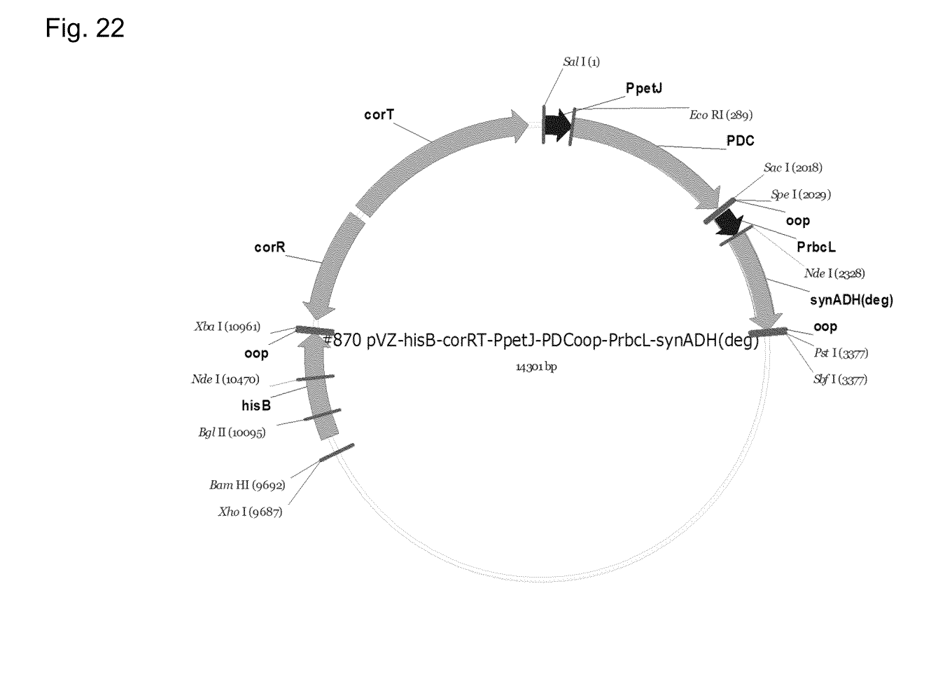

FIG. 22 shows the plasmid map of the first extrachromosomal plasmid #870 harbouring the sequence pVZhisB-corRT-PpetJ-PDCoop-Prbc*-synADH_{deg} for transformation of corRT gene inactivated cyanobacterial cells.

SEQ ID NO. 17 shows the DNA sequence of the plasmid #870 indicating the start and end points of various genes encoded on this plasmid. This plasmid includes from nucleotides 9862 to 10929 the gene hisB, from nucleotides 10930 to 10960 the oop terminator, from nucleotides 11050 to 12162 the gene corR, from nucleotides 12244 to 14172 the gene corT, from nucleotides 2067 to 2325 PrbcL, from nucleotides 2034 to 2066 the oop terminator, from nucleotides 2329 to 3339 the gene coding for synADH(deg), from nucleotides 3340 to 3370 the oop terminator, from nucleotides 293 to 1993 the gene coding for PDC enzyme, and from nucleotides 1 to 288 PpetJ.

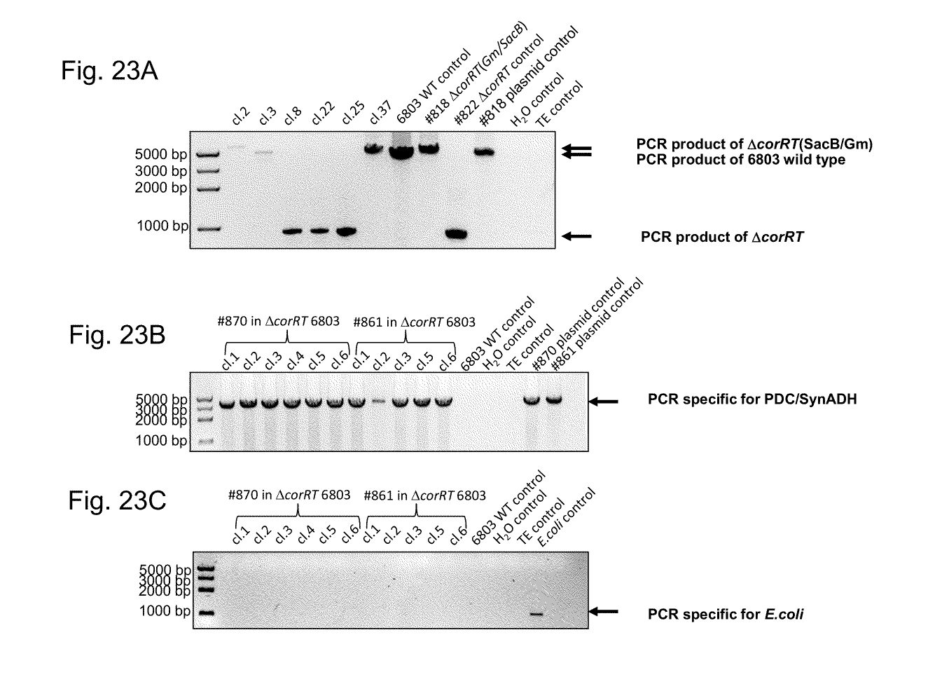

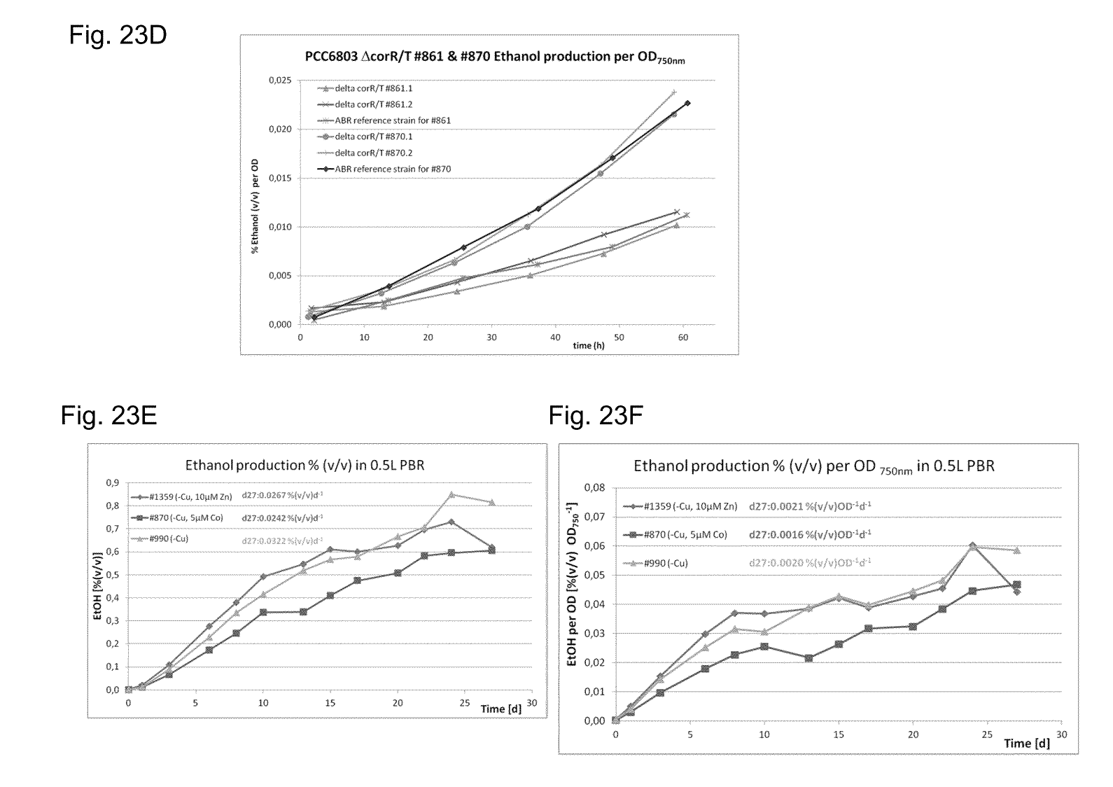

FIGS. 23A to 23F show various DNA agarose gels and graphs depicting ethanol production rates associated with genetically enhanced cyanobacterial cells including a first gene inactivation in the corT gene and also harbouring first extrachromosomal plasmids.

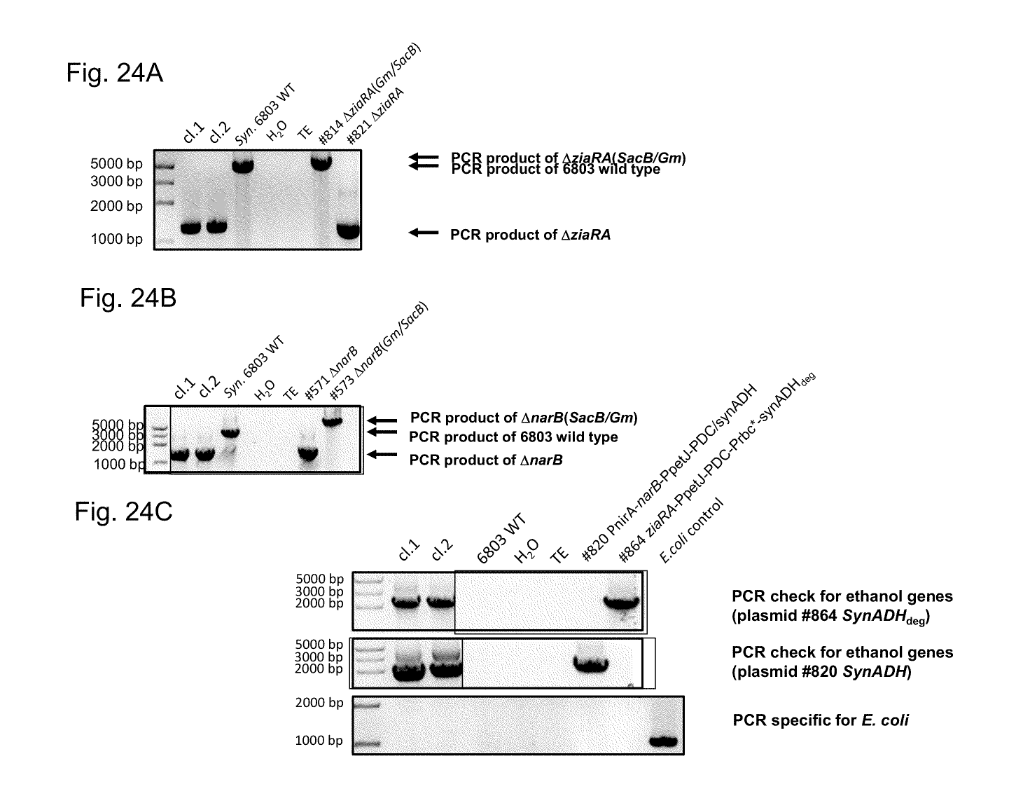

FIGS. 24A to 24C, 25A to 25B and 26A to 26B show various DNA agarose gels and ethanol production rates related to the double knock out ΔziaRA and ΔnarB cyanobacterial Synechocystis sp.PCC 6803 cells.

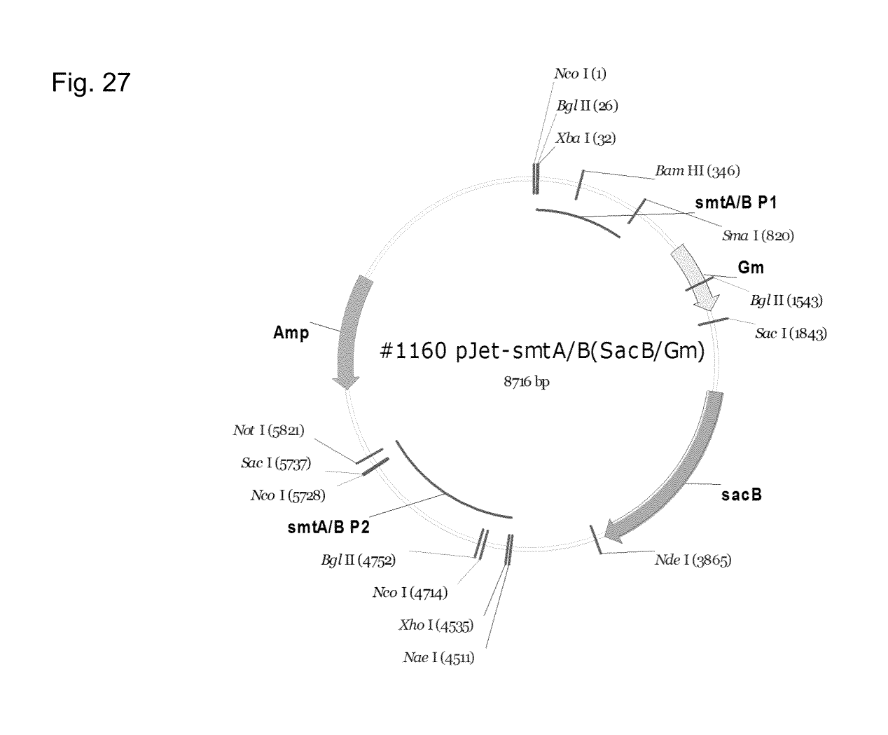

FIG. 27 depicts the plasmid map of the plasmid #1160 including the SacB/Gm cassette and the two platforms smtA/B P1 and smtA/B P2 for homologous integration into the chromosome of Synechococcus PCC 7002 in order to obtain a first gene inactivation by complete deletion of the smtAB genes.

SEQ ID NO. 18 depicts the DNA sequence of the plasmid #1160 indicating the start and end points of various genes encoded on this plasmid. This plasmid contains from nucleotides 4535 to 5820 “smtA/B\P2”, the platform for homologous recombination, from nucleotides 1229 to 1762 the gentamycin resistance cassette, from 6347 to 7204 the Ampicillin resistance cassette, from nucleotides 2371 to 3792 the gene sacB, and from nucleotides 32 to 819 the platform “smtA/B\P1”.

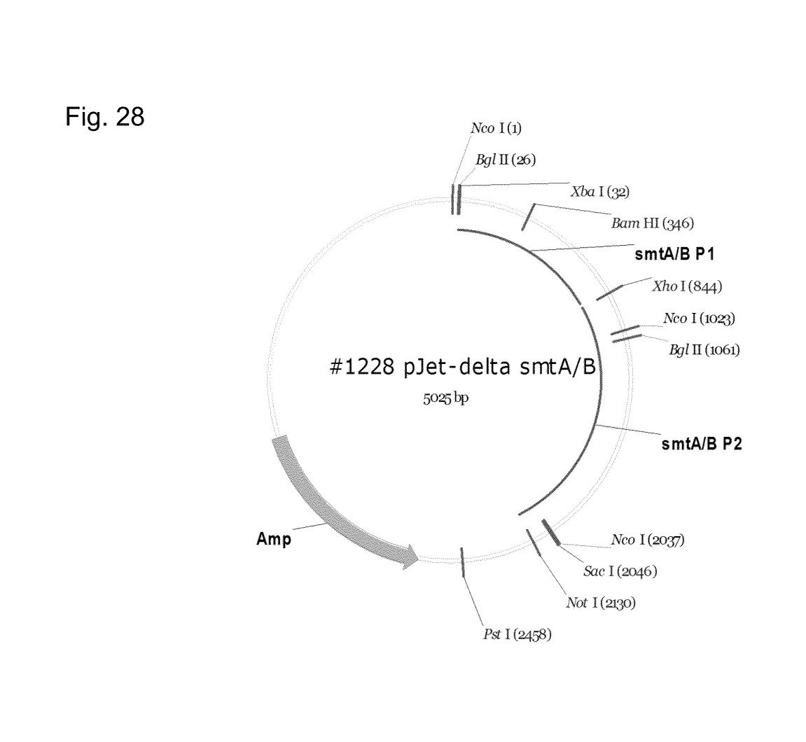

FIG. 28 depicts the plasmid map of plasmid #1228 including the two platforms smtA/B P1 and smtA/B P2 for homologous integration without the SacB/Gm cassette.

SEQ ID NO. 19 shows the DNA sequence of the plasmid #1228 indicating the start and end points of various genes encoded on this plasmid. This plasmid contains from nucleotides 32 to 819 “smtA/B\P1”, the platform for homologous recombination, from nucleotides 2656 to 3513 “Amp”, the Ampicillin resistance gene, and from 844 to 2129 the “smtA/B\P2” platform.

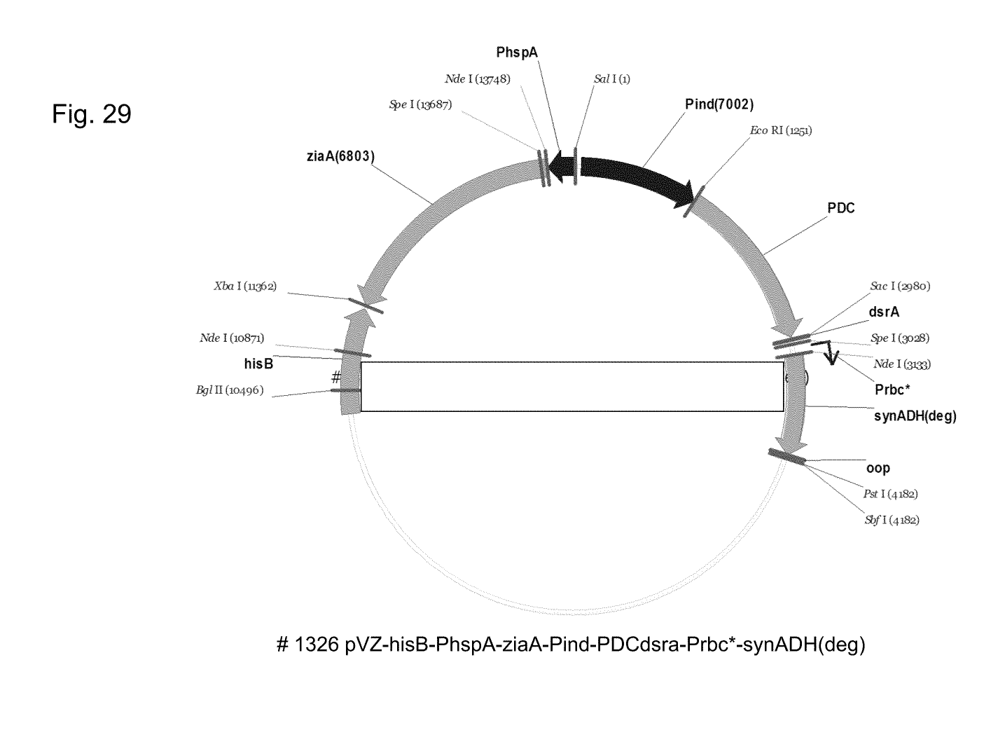

FIG. 29 shows the plasmid map of the extra-chromosomal plasmid #1326 containing the sequence pVZhisB-PhspA-ziaA-P_{ind}-PDCdsrA-Prbc*-synADH_{deg} for ethanol production complementing for the gene inactivation in the smtAB gene of Synechococcus PCC 7002.

In SEQ ID NO. 20 the DNA sequence of the plasmid #1326 is shown indicating the start and end points of various genes encoded on this plasmid. This plasmid includes from nucleotides 10263 to 11330 the gene hisB, from nucleotides 3134 to 4144 the gene coding for synADH(deg), from nucleotides 4145 to 4175 the oop terminator, from nucleotides 2981 to 3026 the terminator sequence dsrA, from nucleotides 1255 . . . 2955 the gene encoding PDC enzyme, from nucleotides 3068 to 3132 Prbc*, from nucleotides 13750 to 14007 PhspA, from nucleotides 11366 to 13749 the gene ziaA(6803), And from nucleotides 57 to 1250 Pind(7002), an inducible promoter for Synechococcus PCC7002.

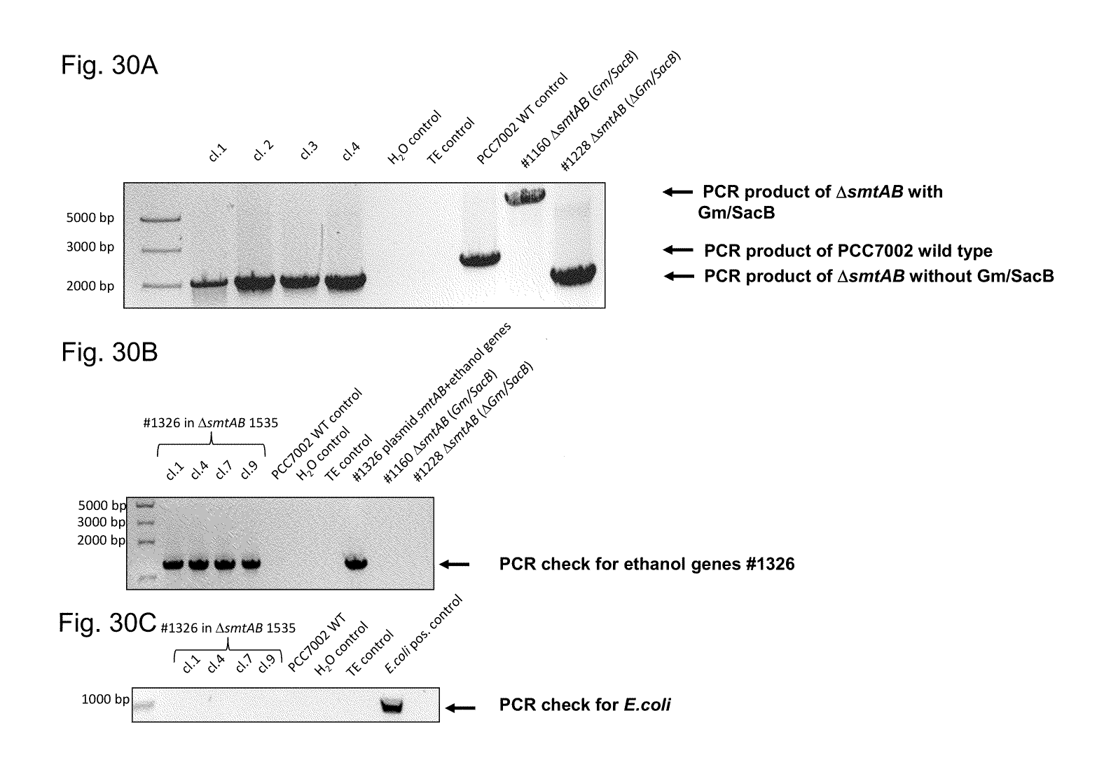

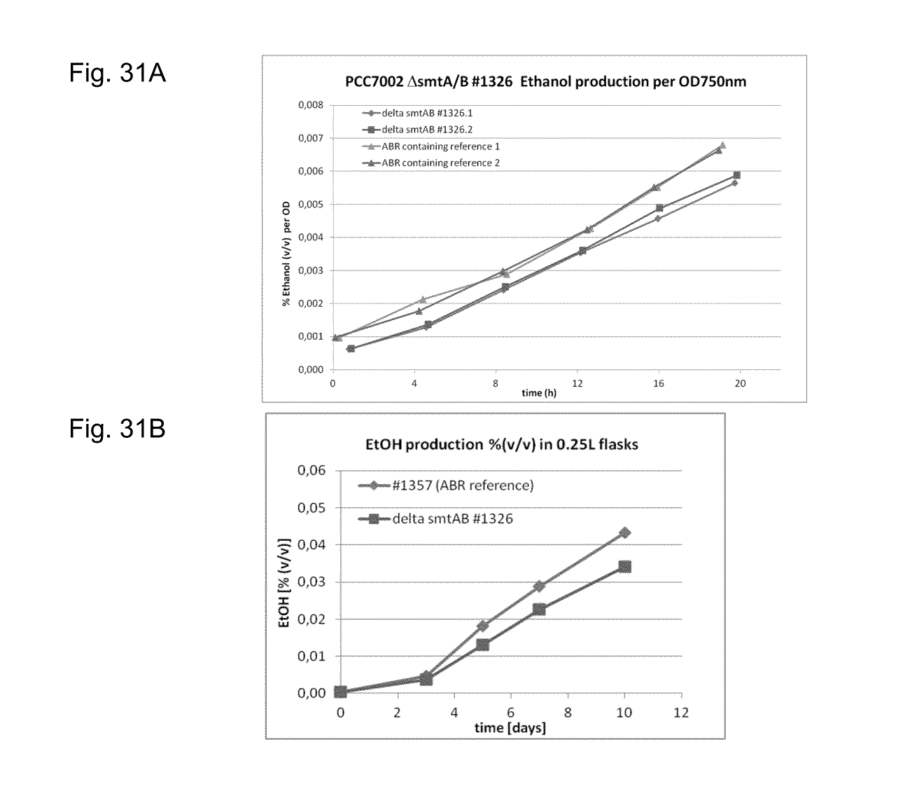

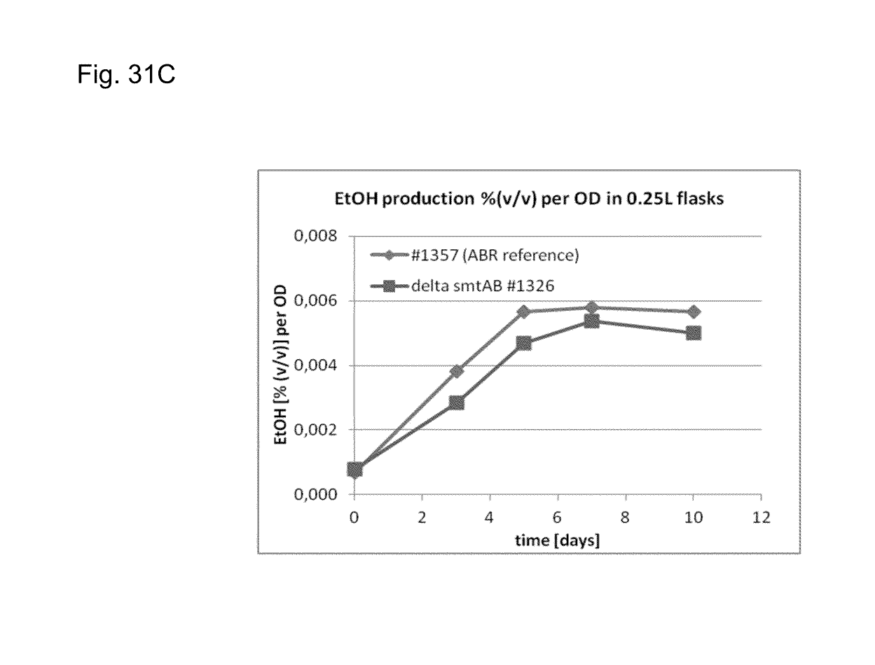

FIGS. 30A to 30C, and 31A to 31C depict various gels and ethanol production rates in relation to Synechococcus PCC 7002 including a gene inactivation in smtAB which is complemented by an extrachromosomal plasmid including ethanologenic genes and ziaRA.

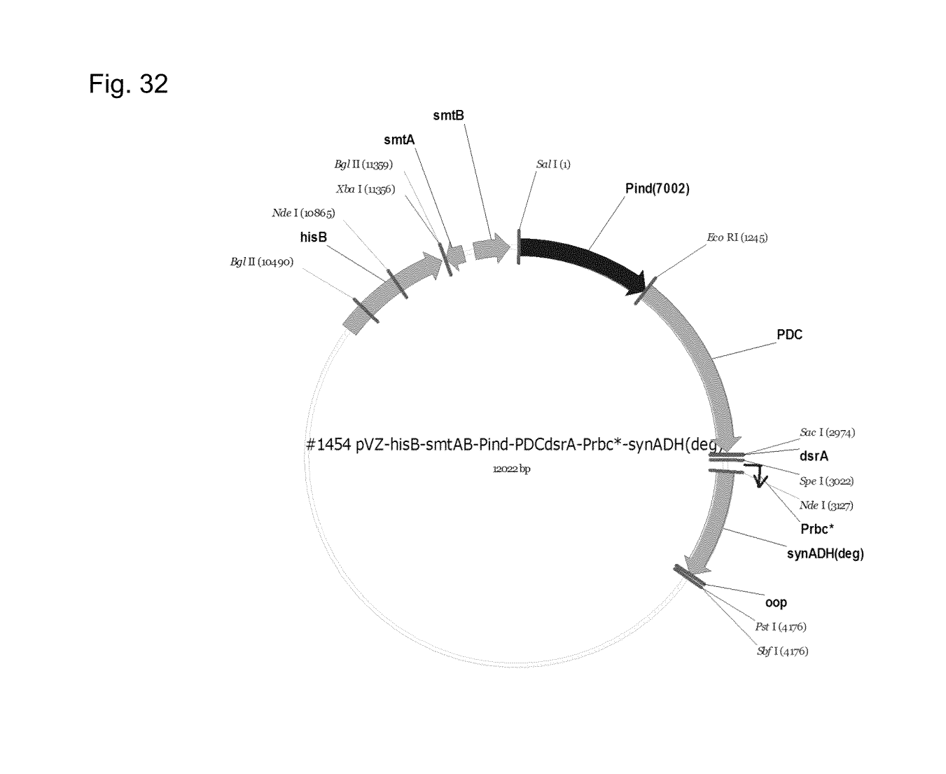

FIG. 32 shows the plasmid map of the plasmid #1454 including the sequence pVZhisB-smtAB-Pind-PDCdsrA-Prbc*-synADH_{deg} for ethanol production and in addition the genes smtAB for complementation of a gene inactivation in the endogenous smtAB genes.

SEQ ID NO. 21 depicts the DNA sequence of the plasmid #1454 indicating the start and end points of various genes encoded on this plasmid. This plasmid contains from nucleotides 11362 to 11532 the gene smtA from Synechococcus PCC7002 (zinc-binding metallothionein), from nucleotides 11635 to 11961 the smtB gene from Synechococcus PCC7002, from nucleotides 3062 to 3126 Prbc*, from nucleotides 1249 to 2949 the gene coding for PDC enzyme, from nucleotides 2975 to 3020 the terminator dsrA, from nucleotides 4139 to 4169 the oop terminator, from nucleotides 3128 to 4138 the gene coding for synADH(deg), from 10257 to 11324 the hisB gene from E.coli and from nucleotides 1 to 1244 Pind(7002), an inducible promoter for Synechococcus PCC7002.

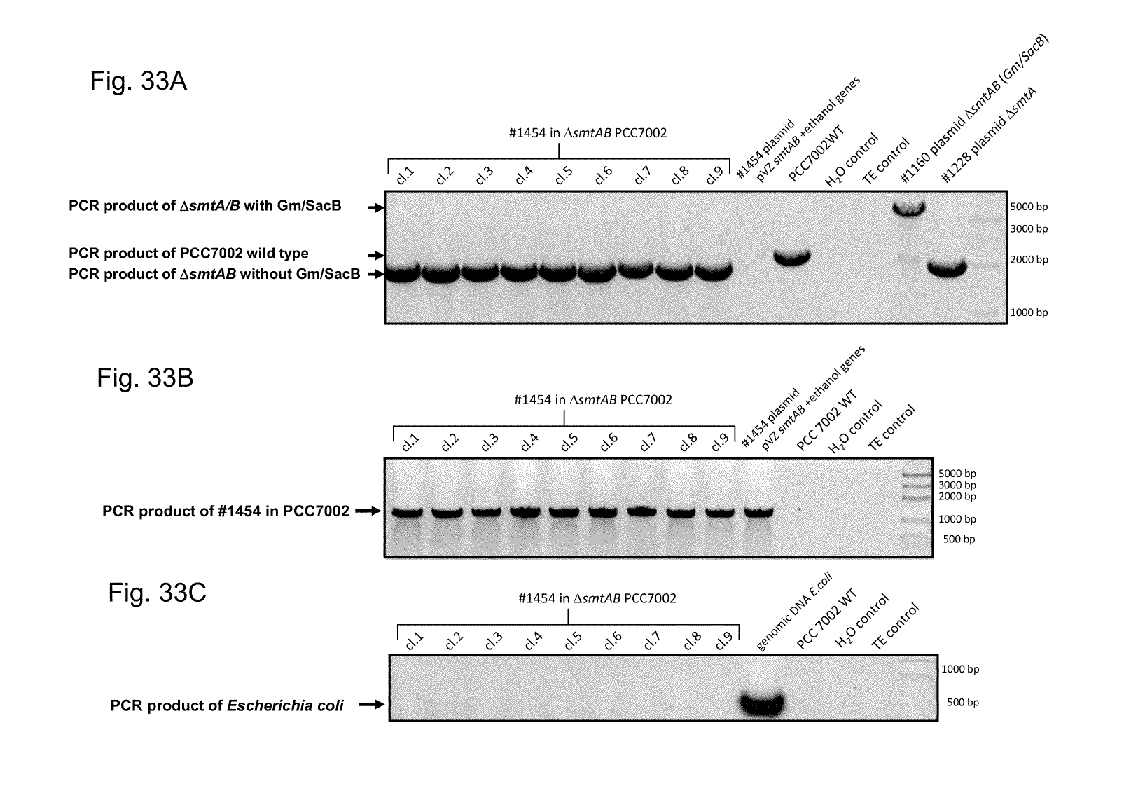

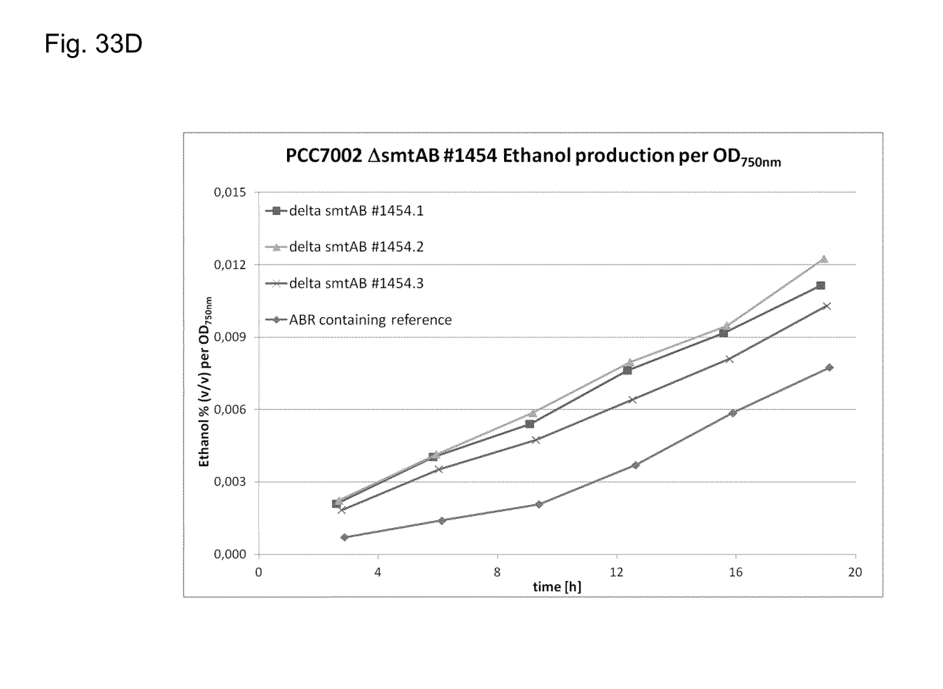

FIGS. 33A to 33D show various gels and ethanol production rates in relation to Synechococcus PCC 7002 including a gene inactivation in the smtAB gene which is complemented by an extrachromosomal plasmid including ethanologenic genes and smtAB.

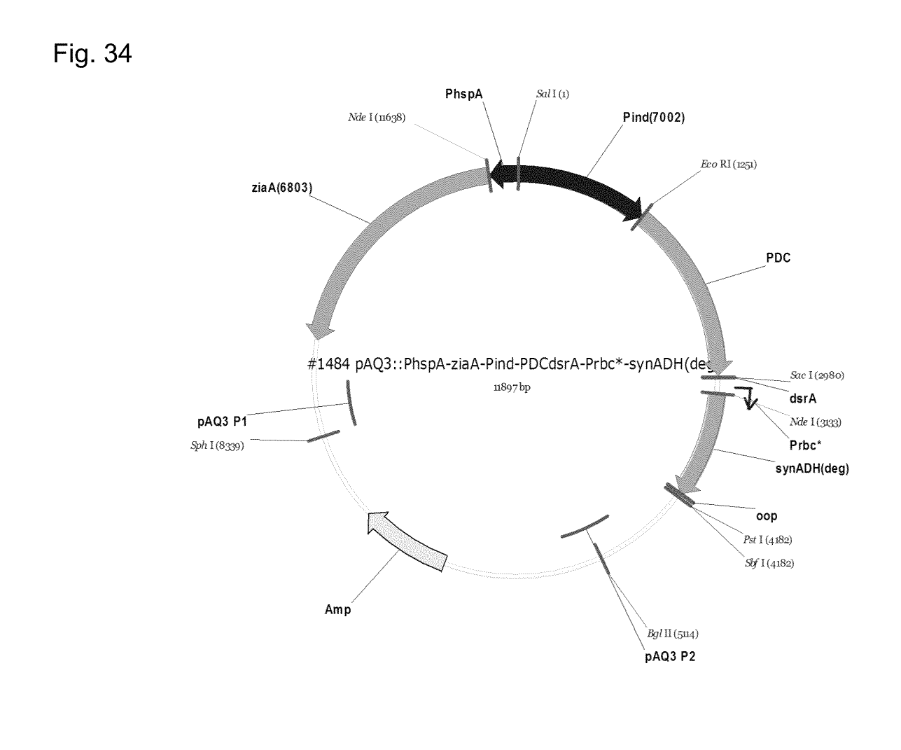

FIG. 34 shows the plasmid map of the pAQ3-integrative ethanologenic plasmid #1484, which includes ethanologenic genes and ziaA for integration into the endogenous Synechococcus PCC 7002 plasmid pAQ3.

SEQ ID NO. 22 depicts the nucleotide sequence of the plasmid #1484. This plasmid contains from nucleotides 4849 to 5417 “pAQ3\P2”, the platform 2 for homologous recombination with pAQ3, from nucleotides 6634 to 7491 “Amp”, the Ampicillin resistance gene, from nucleotides 8359 to 8851 “pAQ3\P1”, the platform 1 for homologous recombination with pAQ3, from nucleotides 9256 to 11639 the gene ziaA(6803), from nucleotides 11640 to 11897 PhspA, the hspA promoter from PCC6803, from nucleotides 3068 to 3132 Prbc*, from nucleotides 1255 to 2955 the gene encoding PDC enzyme, from nucleotides 2981 to 3026 the dsrA terminator, from nucleotides 4145 to 4175 the oop terminator, from nucleotides 3134 to 4144 the gene coding for synADH(deg), the codon-degenerated Adh gene from Synechocystis PCC6803, and from nucleotides 1 to 1250 Pind(7002), an inducible promoter for Synechococcus PCC7002.

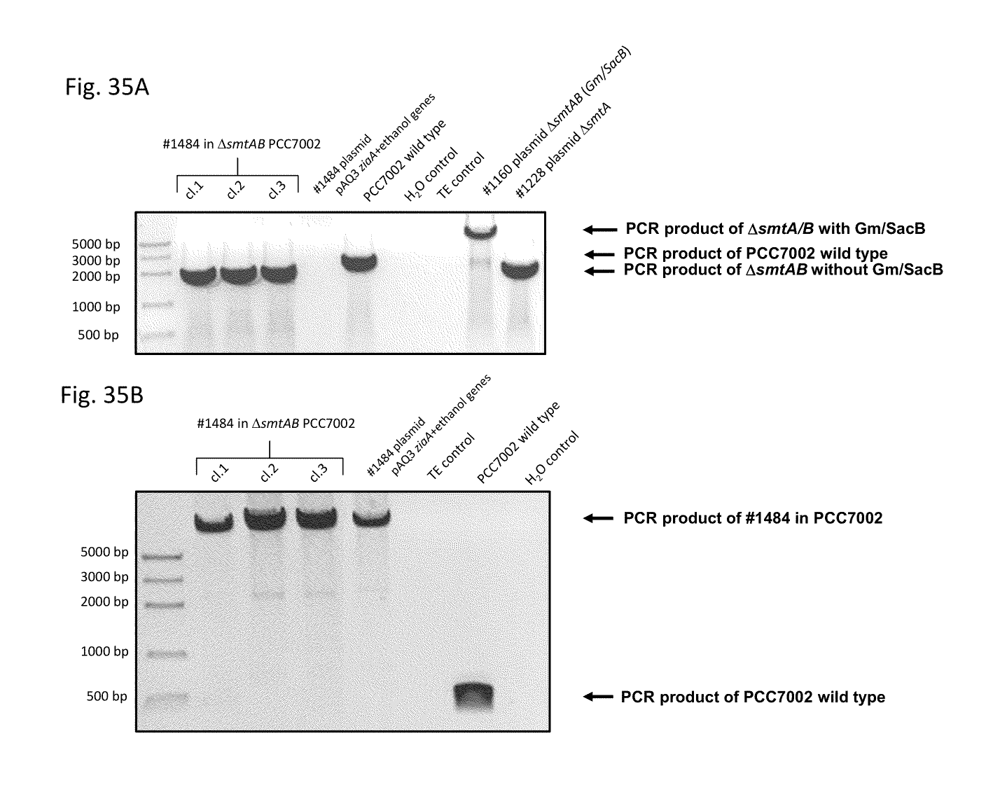

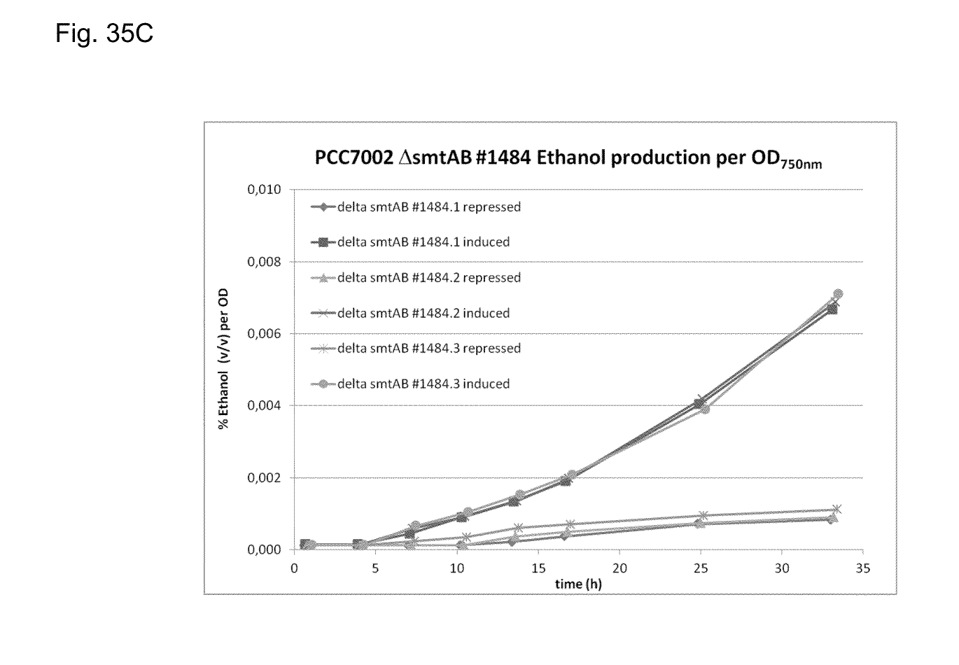

FIGS. 35A to 35C show various DNA agarose gels and graphs related to ethanol production rates for Synechococcus PCC 7002 containing pAQ3 plasmids with incorporated ethanologenic genes and ziaA for complementation of the gene inactivation in smtAB.

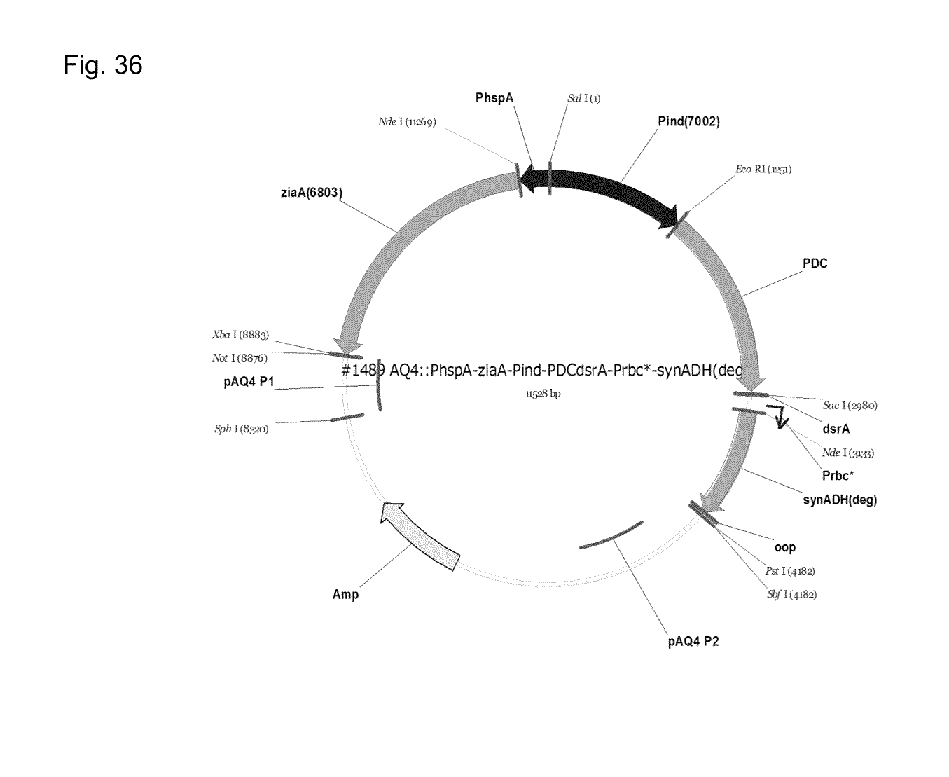

The plasmid map of the suicide plasmid #1489 for integration into the endogenous pAQ4 plasmid containing the sequence pAQ4::PhspA-ziaA-Pind-PDCdsrA-Prbc*-synADH_{deg}. is shown in FIG. 36.

SEQ ID NO. 23 depicts the nucleotide sequence of the plasmid #1489. This plasmid contains from nucleotides 4662 to 5398 “pAQ4\P2”, the platform 2 for homologous recombination with pAQ4, from nucleotides 6615 to 7472 the Ampicillin resistance gene, from nucleotides 8340 to 8879 “pAQ4\P1”, the platform 1 for homologous recombination with pAQ4, from nucleotides 8887 to 11270 ziaA(6803), from nucleotides 11271 to 11528 PhspA, from nucleotides 3068 to 3132 Prbc*, the truncated rbc core promoter based on PrbcL from PCC6803, from nucleotides 1255 to 2955 the gene coding for PDC enzyme, from nucleotides 2981 to 3026 the dsrA terminator, from nucleotides 4145 to 4175 the oop terminator, from nucleotides 3134 to 4144 the gene coding for synADH(deg) and from nucleotides 1 to 1250 Pind(7002), an inducible promoter for Synechococcus PCC7002.

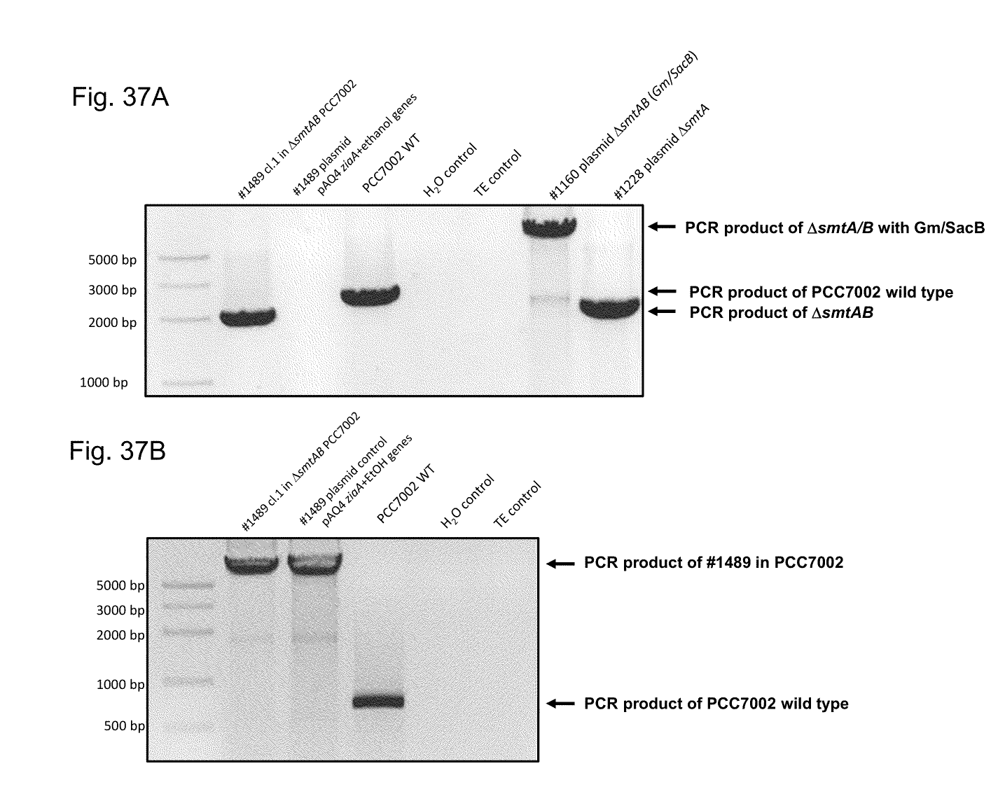

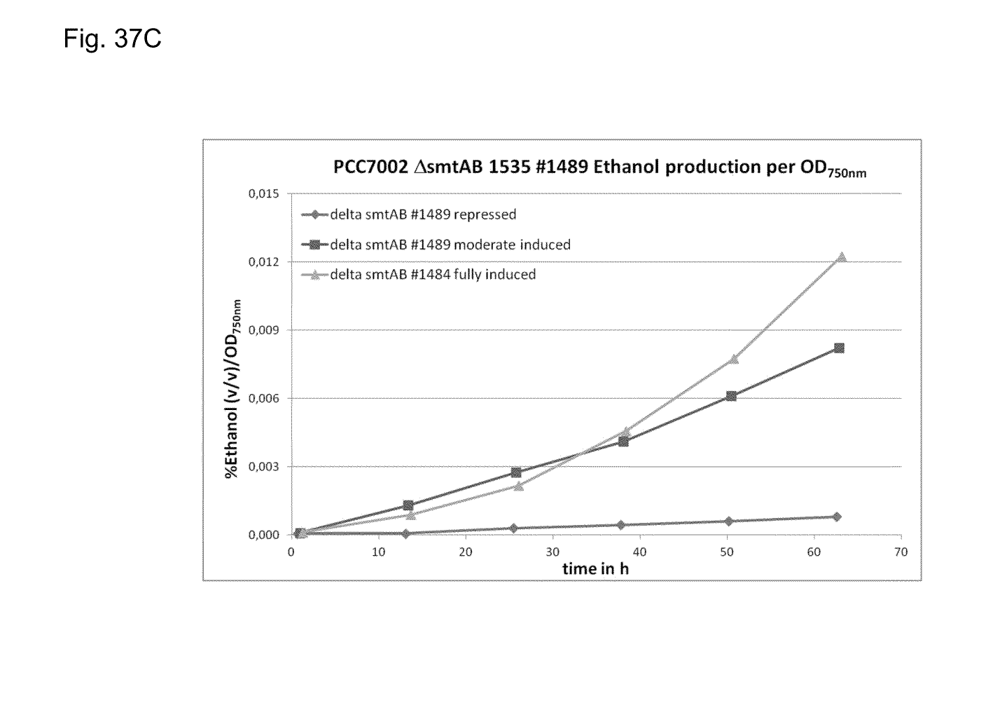

FIGS. 37A to 37C show various DNA agarose gels and graphs related to ethanol production rates for Synechococcus PCC 7002 containing pAQ4 plasmids with incorporated ethanologenic genes and ziaA for complementation of the gene inactivation in smtAB.

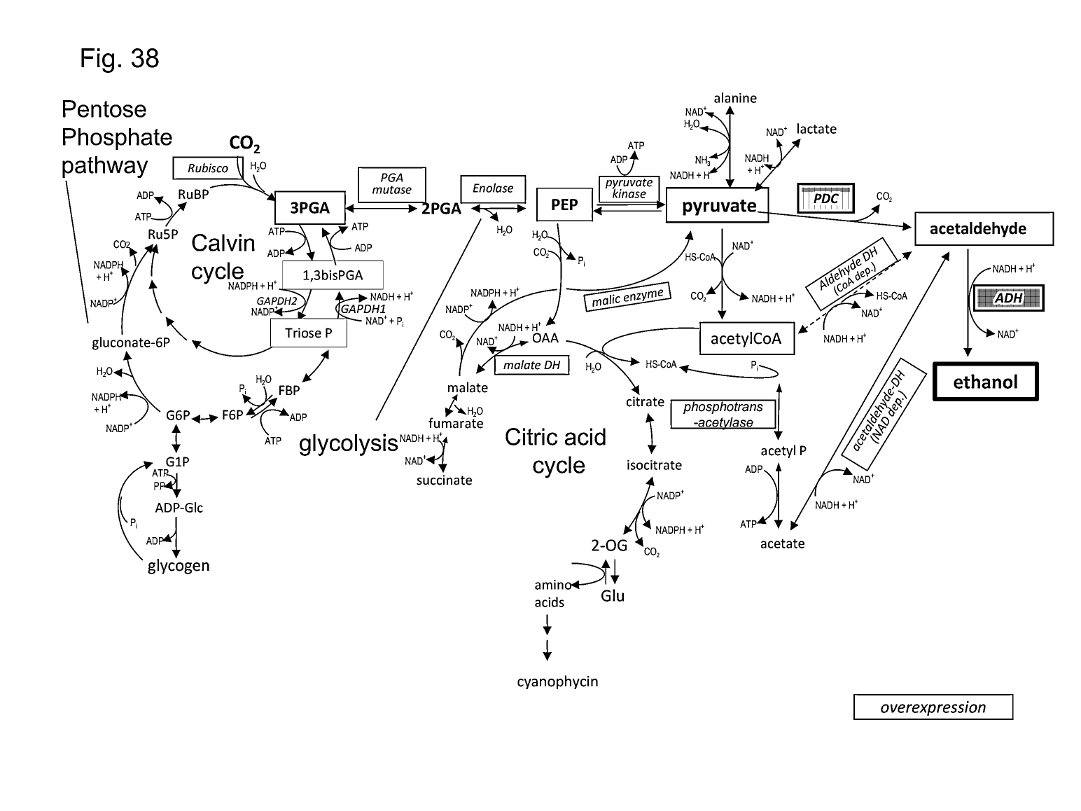

FIG. 38 schematically depicts general metabolic pathways in cyanobacteria. The enzymes of the glycolysis pathway, of the citric acid cycle and of the fermentation pathway, which are marked by squared boxes are prime candidates for enzymes, which are encoded by second production genes on second extrachromosomal plasmids according to some embodiments of the invention.

### DETAILED DESCRIPTION OF EXPERIMENTAL RESULTS

I. Construction of an Antibiotic-Resistance-Cassette Free Expression System Based on the pyrF Gene Inactivation for Generation of Ethanologenic Hybrids in Synechocystis Sp.PCC 6803
I.1 Unsuccessful Attempt to Completely Delete the pyrF Gene Via Homologous Recombination

In a first attempt to inactivate the pyrF gene in Synechocystis sp. PCC6803 it was decided to partially delete the Synechocystis pyrF gene (sll0858), which should lead to the inactivation of the orotidine-5′-monophosphate decarboxylase encoded by this gene. In order to partially delete the pyrF gene a plasmid (first recombinant nucleic acid sequence) was created which is flanked by pyrF sequences denoted “pyrF′” and “′pyrF” and also contains a first selectable gene, conferring biocide resistance, namely resistance to kanamycin, which is denoted as “Km” in the plasmid in FIG. 14A left-hand side step 1). In a second step as shown on the right side of FIG. 14A, it was planned to replace this first recombinant nucleic acid sequence by a second recombinant nucleic acid sequence via homologous recombination. This second sequence includes first production genes encoding Pdc and Adh enzymes as well as the pyrF gene copy from Anabaena PCC 7120. Therefore, this planed strategy did not involve the use of a first extrachromosomal plasmid.

Although uracil was supplemented into the growth medium, no complete segregation of the cyanobacteria could be obtained via the above mentioned gene inactivation strategy. This means that the cyanobacteria obtained via the transformation and which were selected according to their resistance to the antibiotic kanamycin contained gene inactivations in some gene copies of the pyrF gene but also retained wild type copies of the functional pyrF gene. This is shown in the DNA agarose gel of FIG. 14B. The lane denoted as “WT” shows the wild type pyrF gene. The lanes denoted with the numbers 1 to 4 show the DNA from genetically enhanced cyanobacteria which were obtained via the gene inactivation strategy depicted in FIG. 14A. It is clearly visible that all genetically enhanced cyanobacteria of the lanes 1 to 4, apart from the exogenous second nucleic acid sequence, still retain wild type copies of the pyrF gene. This result clearly indicates that the gene inactivation strategy employed was not sufficient, despite the fact that the first essential factor, uracil was supplemented in the growth medium. This clearly shows that uracil as a first essential factor cannot compensate efficiently the pyrF gene inactivation to promote the growth of the cyanobacteria if present in the growth medium.

1.2 SacB/Cm Mediated Gene Inactivation of pyrF and Complementation of ΔpyrF Via the First Extrachromosomal Plasmid

In a second attempt, the first extrachromosomal plasmid pVZ-pyrF₇₁₂₀-PpetJ-PDC-P_{rbc*}-SynAdh was introduced into partially segregated ΔpyrF(SacB/Cm) cells. Afterwards the genetically enhanced cells were fully segregated by subjecting the cells to increased concentrations of the biocide Chloramphenicol as the first selectable marker. This was possible due to the introduced second pyrF gene copy from Anabaena variabilis encoded on the first extrachromosomal plasmid that complements the lack of the genomic pyrF gene. FIG. 14C shows in the upper panel the results of a primer pair specific PCR for wildtype pyrF and inactivated ΔpyrF(SacB/Cm) gene. This panel shows that four genetically enhanced cyanobacterial clones including the plasmid #802 named #802.1 to #802.4 could be isolated which harbour a complete gene inactivation of the wild type pyrF gene. The result of a further PCR analysis with primers specific for the wild type pyrF gene in Synechocystis sp. PCC 6803 was carried out and the result is presented in the middle panel of FIG. 14C. This “wild type specific PCR” clearly shows that wild type copies of the Synechocystis pyrF gene are not present in the any of the isolated clones #802.1 to #802.4. The lower panel in FIG. 14C shows the result of a primer specific PCR reaction with primers specific for the first extrachromosomal plasmid #802. As shown, this plasmid is present in the clones #802.1 to #802.4 isolated via this gene inactivation strategy.

1.3 Sucrose Mediated Removal of sacB/Cm Cassette from ΔpyrF Cyanobacterial Cells

FIG. 14D shows a PCR analysis evidencing that after selection of a complete segregated ΔpyrF(sacB/Cm) #802 clone including the extra chromosomal plasmid #802 on sucrose via transformation with a second recombinant nucleic acid ΔpyrF during the method steps i′3) to i′5) the first recombinant nucleic acid ΔpyrF(SacB/Cm) is replaced by subjecting the cells to the counterselectable marker sucrose. PCR analyses of several Cm sensitive ΔpyrF #802 clones after sucrose selection revealed a successful removal of the SacB/Cm cassette. In the upper panel of FIG. 14D the results of a primer pair specific PCR for the pyrF locus was used leading to a small PCR product (˜1600 bp) for the intended ΔpyrF gene inactivation, a slightly larger PCR product of about 2000 bp for the wild type and a substantial larger PCR product of about 5500 bp for clones without removed SacB/Cm cassette ΔpyrF(SacB/Cm). In the middle panel of FIG. 14D the results of a primer pair specific PCR for ethanologenic genes of plasmid #802 indicating the presence of the ethanologenic pVZ plasmid are shown. The specific primer pair used for the PCR shown in the lower panel of FIG. 14D indicates that no wild type pyrF allele is present in respective clones. Red arrows indicate genetically enhanced cyanobacterial colonies (clone 1, 14 and 18) with the intended genotype (removed sacB/Cm cassette).

1.4 Ethanol Production Rates of Cyanobacterial Cells with a First Gene Inactivation in the pyrF Gene Harbouring the First Extrachromosomal Plasmid Including Ethanologenic Genes and the pyrF Gene

FIG. 14E shows a graph of the ethanol production rate of some of the clones #802.1 and #802.2 isolated via the above-described gene inactivation strategy. The clones denoted #550.1 and #550.2 are Synechocystis cells comprising a conventional extrachromosomal plasmid #550 with a Gentamycin conferring resistance gene. The cyanobacterial Synechocystis sp. PCC 6803 cells were grown in BG11 medium over a period of time of at least 18 hours and the ability to produce ethanol was tested by online gas chromatography experiments. The online gas chromatography experiments were done by measuring the ethanol rates by gas chromatography over 18 hours of cultivation in an illuminated GC vial(100 μE*m⁻²*s⁻¹). The graph clearly shows that similar production rates were obtained by culturing the antibiotic resistance free cyanobacteria of the invention compared to conventional cyanobacteria harbouring ethanologenic enzymes. In particular an ethanol production rate of 0.012% (v/v)/d*OD₇₅₀ₙₘ was obtained.

II. Construction of an Antibiotic-Resistance-Cassette Free Expression System Based on the leuB Gene Inactivation for Generation of Ethanologenic Hybrids in Synechocystis Sp.PCC 6803

FIG. 15A schematically depicts a successful gene inactivation strategy according to one variant of the method of the invention for the production of a first gene inactivation in the leuB gene of Synechocystis sp. PCC 6803. In a first step denoted “1)” a first exogenous nucleic acid sequence comprising a Chloramphenicol resistance-conferring gene denoted “Cm” as a first selectable gene and also the sacB gene as a second counterselectable gene are introduced into the Synechocystis cells via homologous recombination thereby creating a first gene inactivation in the leuB gene. In a second step denoted “2)” the first extrachromosomal plasmid harbouring a leuB gene from Anabaena variabilis PCC 7120 and additionally containing first production genes encoding Pdh and Adh enzymes under the transcriptional control of the petJ promoter is introduced into the cells. After having selected for cyanobacterial cells, which are completely segregated with regard to the first gene inactivation, so that the cells do not contain functional wild type copies of the leuB gene anymore, the last step 3) can be carried out. This step 3) involves the transformation of the cells from step 2) with a second recombinant nucleic acid sequence via homologous recombination. This second recombinant nucleic acid sequence lacks both the “Cm” gene for conferring Chloramphenicol resistance and the sacB gene, conferring sensitivity to sucrose, thereby creating a cyanobacterium with a first gene inactivation in the leuB gene, which in additional also lacks a biocide conferring resistance gene. “leuB′” denotes an inactivated truncated variant of the leuB gene. Similarly to the pyrF gene inactivation, the inventors also encountered serious problems in creating a fully segregated first gene inactivation in the leuB gene. A complete segregation of the first gene inactivation in the leuB could only be accomplished after the first extrachromosomal plasmid comprising the leuB gene and additionally first production genes for the production of ethanol were introduced into the cells.

II.1 SacB/Cm Mediated Gene Inactivation of leuB and Introduction of the First Extrachromosomal Plasmid

FIG. 15B shows a DNA agarose gel evidencing the complete genetic segregation of the Synechocystis sp. PCC 6803 cells with a first gene inactivation in the leuB gene after step 2) denoted in FIG. 15A. After the transformation with the first recombinant nucleic acid harbouring a Cm and sacB coding gene, the first extrachromosomal plasmid #570 is introduced into the cells, which harbour the ethanologenic genes coding for Pdc and Adh enzymes and which also contains a gene coding for LeuB enzyme of Anabaena variabilis PCC 7120. The FIG. 15B shows the PCR segregation test of four different Δleu(SacB/Cm) colonies named ΔleuB(SacB/Cm)#570.1 to ΔleuB(SacB/Cm)#570.4 after conjugational transfer of the ethanologenic plasmid pVZ-leuB₇₁₂₀ (#570). The upper panel shows the results of a primer pair specific PCR for wild type leuB and inactivated ΔleuB(SacB/Cm) gene. It can be seen that the colonies ΔleuB(SacB/Cm)#570.1, ΔleuB(SacB/Cm)#570.2 and ΔleuB(SacB/Cm)#570.3 are completely segregated with regard to the first gene inactivation and only contain the ΔleuB(SacB/Cm) construct, but no wild type copy of the leuB gene anymore, whereas the colony ΔleuB(SacB/Cm)#570.4 still harbors wild type copies of the leuB gene. The middle panel shows the result of a primer pair specific PCR exclusively for wild type leuB gene and the lower panel depicts the results of a primer pair specific PCR for the ethanologenic plasmid #570, which is present in all four tested colonies.

II.2 Sucrose Mediated Removal of sacB/Cm Cassette from ΔleuB Cyanobacterial Cells

FIG. 15C shows an agarose DNA gel evidencing that after selection on sucrose as the counterselectable marker after the transformation with the second recombinant nucleic acid ΔleuB in the method step 3) shown in FIG. 15A, the first recombinant nucleic acid ΔleuB(SacB/Cm) is replaced. Red arrows indicate colonies (clone 2, 4, 19 and 21) which exhibit the intended genotype (removed SacB/Cm cassette, no wild type copy of leuB and only ΔleuB construct). The upper panel shows the results of a primer pair specific PCR for wild type leuB and inactivated ΔleuB gene, the middle panel shows the results of a primer pair specific PCR for the ethanologenic plasmid #570 and the lower panel depicts the results of a primer pair specific PCR exclusively for wild type leuB gene.

II.3 Ethanol Production Rates of Cyanobacterial Cells with a First Gene Inactivation in the leuB Gene Harbouring the First Extrachromosomal Plasmid Including Ethanologenic Genes and the leuB Gene

FIG. 15D depicts a graph of a long-term ethanol production experiment of another completely segregated Synechocystis sp. PCC 6803 colony #584.2 harbouring the ethanologenic plasmid pVZ #584. The plasmid #584 includes a petJ promoter controlling the transcription of the leuB gene as the only difference to the plasmid #570, which includes the endogenous Anabaena promoter operably linked to the leuB gene of Anabaena. This colony also includes a first extrachromosomal plasmid #584 which is a pVZ plasmid containing the leuB gene of Anabaena PCC 7120 under the control of the petJ promoter. Furthermore, Pdc and Synechocystis Adh encoding genes are present on this plasmid which are also transcriptionally controlled by the petJ promoter.

The long-term ethanol production experiment was run for 70 days in 12 h/12 h day/night cycles and an average ethanol production rate of 0.0164% (v/v) per day over a time period of 70 days was determined by GC measurements of the head space of a 0.5 ml sample taken from the cultures., This production rate is similar to conventional cyanobacterial strains harbouring biocide resistance-conferring genes.

III. Construction of an Antibiotic-Resistance-Cassette Free Expression System Based on the narB Gene Inactivation for Generation of Ethanologenic Hybrids in Synechocystis Sp.PCC 6803
III.1 Gene Inactivation of the narB Gene Using a First Nucleic Acid with SacB/Cm

FIG. 16A shows the results of a PCR analyses of different Cm sensitive ΔnarB(SacB/Cm) colonies after sucrose selection as a counterselectable marker. The used primer pair is specific for wild type narB and inactivated ΔnarB gene. The colonies ΔnarB cl.1-cl.9 exhibit the intended genotype, a completely segregated first gene inactivation in the narB gene, leading to a loss of the ability to use nitrate as a sole nitrogen source (removed SacB/Cm cassette and no wild type narB gene). Therefore ammonium had to be supplemented to the growth medium of these cells in order to complement for the first gene inactivation. The lane denoted “6803 WT control” shows the DNA signal of the wild type narB gene in Synechocystis sp. PCC 6803.

III.2 Introduction of the First Extrachromosomal Plasmid with the narB Gene and Ethanologenic Genes

FIG. 16B shows an agarose gel showing the results of a primer specific PCR for eight successful complemented ΔnarB colonies by conjugation with ethanologenic pVZ-narB plasmids #819 and #820 (product of method step ii). Four colonies denoted ΔnarB #819 transformed with the plasmid #819 and four colonies denoted ΔnarB #820 transformed with the plasmid #820 were analyzed. The used primer pairs are specific for the ethanologenic gene cassette present in the plasmids #819 and #820, respectively.

III.3 Short Term Ethanol Production Rates of Cyanobacterial Strains with a First Gene Inactivation in the narB Gene Including a First Extrachromosomal Plasmid with a Copy of the narB Gene and Ethanologenic Genes

Both graphs in FIG. 16C show the ethanol production rates of four different genetically enhanced Synechocystis strains denoted #820.1, #820.2, #819.1 and #819.2 all harbouring a first gene inactivation in the narB gene, which were grown in a BG11 growth medium which was free of ammonium or nitrite as alternative sources for nitrogen. The genetically enhanced cyanobacterial cells either included the ethanologenic pVZ plasmids #819 or #820, which are pVZ plasmids including a narB gene under the transcriptional control of the nirA* promoter, which is a truncated and slightly modified version of the original nirA promoter including all regulatory elements, in particular the core promoter region between the nucleotides-10 and 35. The plasmid #819 also includes a Pdc encoding gene under the control of the petJ promoter and a Synechocystis Adh encoding gene under the control of the rbc* promoter, only including the core promoter from −35 to the start codon ATG. Plasmid #820 includes a pVZ plasmid comprising the narB gene under the transcriptional control of the nirA* promoter and in addition the Pdc and Synechocystis Adh encoding genes both are under the transcriptional control of the petJ promoter. The graphs denoted with #550.1 and #550.2 and #309.1 and #309.2 are conventional cyanobacteria including the plasmids #550 and #309 harbouring a Gentamycin resistant cassette. It can clearly be seen that by using these genetically enhanced cyanobacteria similar ethanol production rates or even higher production rates can be achieved compared to conventional cyanobacterial cells.

III.4 Long Term Ethanol Production Rates of Cyanobacterial Strains with a First Gene Inactivation in the narB Gene Including a First Extrachromosomal Plasmid with a Copy of the narB Gene and Ethanologenic Genes

In FIG. 16D the ethanol production and in FIG. 16E the ethanol production normalized on OD at 750 nm of a Synechocystis ΔnarB strain complemented with the narB-ethanologenic plasmid #820 in comparison to a wild type with conventional ethanologenic plasmid “WT #309” is depicted. Both were grown in pH controlled 0.5 L photobioreactors (PBR) in mBG11 growth medium without copper aerated with CO₂ enriched air with (10% CO₂). Plasmid maintenance of ΔnarB #820 (pVZhisB-PnirA*-narB6803-PpetJ-PDC/synADH) is self-sustained in the presence of nitrate as sole nitrogen source (as it is the case in usual mBG11 medium) whereas for the conventional reference strain #309 (pVZ325-PpetJ-PDC/synADH) gentamycin was added to maintain the ethanologenic plasmid over the duration of the growth experiment. Plasmid #820 is a self-replicating pVZ plasmid comprising the narB gene under the transcriptional control of the nirA* promoter (truncated version of the native nirA promoter from Synechocystis) for restoration of the nitrate usability. In plasmid #820 and #309 Pdc and synAdh encoding genes are co-transcribed under control of the copper-depletion inducible petJ promoter.

In FIG. 16F the measured PDC activities at four different time points within the cultivation experiment are shown. The presented ethanol production rates as well as the detected PDC activities for both genetically enhanced cyanobacteria are very similar over the analyzed timeframe. The long-term ethanol production experiment was run in 14 h/10 h day/night cycles and an average ethanol production rate of 0.0288% (v/v) per day over a time period of 25 days was measured, which is similar to conventional cyanobacterial strains harbouring biocide resistance-conferring genes.

III.5 Comparison of Ethanol Production Rates of Genetically Enhanced Cyanobacterial Strains Synechocystis Sp.PCC 6803 Harboring First Gene Inactivations in the leuB, pyrF and narB Genes

The cyanobacterial strains described above in the sections I. II. and III. were used for long term ethanol production experiments as outlined below.

In FIG. 16G the ethanol production and in FIG. 16H the ethanol production per OD of the ABR free strains ΔleuB #570 (pVZleuB-PpetJ-PDC/synADH), ΔpyrF #802 (pVZpyrF-PpetJ-PDC-Prbc*-synADH_{deg}) and ΔnarB #820 (pVZhisB-PnirA*-narB-PpetJ-PDC/synADH) are presented. This long term experiment was running over a time frame of about 100 days in 50 ml Erlenmeyer flasks in mBG11 medium. In all three ABR-free strains the plasmid maintenance is self-sustained due to the essential genes leuB and pyrF as well as the conditionally essential narB gene which were transferred from the usual location on the chromosome onto the extra-chromosomal plasmids #570, #802 and #820. The expression of the ethanologenic genes encoded on the above described plasmids #570, #802 and #820 is regulated via the petJ promoter. In the copper-free BG11 medium the ABR free strains show very similar and stable ethanol production rates, also after every medium exchange (indicated by arrows).

Furthermore after 84 days of cultivation, samples were taken and analyzed for PDC activity. In FIG. 16I the measured PDC activities are shown which indicate a high PDC activity still after 84 days. According to that the cyanobacteria are able to maintain the ABR free extra-chromosomal plasmids over the entire time of cultivation without addition of any compound that conventionally assures plasmid maintenance of ABR based selection systems.

These data clearly indicate that the genetically enhanced cyanobacterial strains harbouring the first extrachromosomal plasmids and the first gene inactivation are able to produce ethanol over a long period of time of at least 100 days.

IV. Construction of an Antibiotic-Resistance-Cassette Free Expression System Based on Zn²⁺ Sensitive Phenotype Via the ziaRA Gene Inactivation for Generation of Ethanologenic Hybrids in Synechocystis Sp.PCC 6803

FIG. 17A schematically depicts the gene inactivation strategy employed for the generation of a genetically enhanced cyanobacterium harbouring a first gene inactivation in the ziaRA genes by a complete deletion of the ziaRA genes, leading to sensitivity against Zn²⁺. It is shown that in a first step “1)” a first recombinant nucleic acid sequence comprising two endogenous Synechocystis sp. PCC 6803 genes, namely slr0797 and sll0790 for homologous recombination are introduced into this plasmid and are flanking two genes, a first selectable marker gene conferring Gentamycin resistance denoted with “Gm” and a second counterselectable “sacB” gene conferring sensitivity to sucrose. The introduction of this first exogenous nucleic acid sequence via homologous recombination into the genome of PCC 6803 results in a first gene inactivation of the ziaRA genes. In a second step “2)”, driven by sucrose selection a second exogenous nucleic acid comprising only the flanking sequences of the ziaRA genes (P1 and P2) used as platforms for homologous recombination is introduced into the genome of the cyanobacterium thereby removing the biocide conferring resistance gene along with the counterselectable marker sacB. In a third step denoted “3)”, the first extrachromosomal plasmid harbouring genes encoding the Pdc and Adh enzyme under the control of the petJ promoter and also containing the endogenous ziaRA gene segment (including the native promoters PziaR and PziaA) is introduced in the cyanobacterium. The selection for the cyanobacterium harbouring the first extrachromosomal plasmid is driven by addition of 5 μM Zn² to the growth medium.

IV.1 Complete Genetic Segregation of the Cyanobacterial Strains Harbouring a First Gene Inactivation in the ziaRA Genes

FIG. 17B shows a DNA agarose gel evidencing that complete segregation of the ziaRA gene inactivated cyanobacterial cells was achieved. The lane denoted “6803 WT control” shows the signal of the wild type ziaRA gene which is not existent in any of the colonies “cl.2”, “cl.4” and “cl.17” isolated from the gene inactivation procedure. Furthermore, this DNA agarose gel shows that the first extrachromosomal plasmid #864 comprising Pdc and Adh enzyme is present in all colonies. This first extrachromosomal plasmid also contains the ziaRA genes (including the native promoters PziaR and PziaA) for restoration of the zinc tolerance. The extrachromosomal plasmid contains the sequence pVZ-ziaRA-PpetJ-PDC-Prbc*SynADH_{deg}. SynADH_{deg} denotes a degenerated DNA sequence having a sequence identity of 61% to the wild type gene coding for Synechocystis sp. PCC 6803 Adh enzyme. In particular this gene codes for a SynAdh enzyme with identical amino acid sequence compared to the wild type protein, but wherein all of the wobble bases are replaced in order to reduce the risk of homologous recombination with the genomic SynAdh coding gene. This SynADH_{deg} gene is under the transcriptional control of the truncated rbc* promoter, and the gene coding for Pdc enzyme is under the transcriptional control of the petJ promoter. The upper panel shows the results of a primer pair specific PCR for ethanologenic plasmid #864 and the lower panel the results of a primer pair specific PCR for genomic ziaRA locus, which is not present in the colonies “cl.2”, “cl.4” and “cl.17” and for the ΔziaRA locus, which can be found in all three colonies. In the colonies denoted cl.2, cl.4 and cl.17 the amplified ΔziaRA locus has a smaller size than the ΔziaRA(Gm/SacB) locus or the wild type allele of the ziaRA locus due to the first gene inactivation, which involves a complete deletion of the ziaRA genes.

IV.2 Ethanol Production Rates of Genetically Enhanced Cyanobacteria with a First Gene Inactivation in the ziaRA Genes Including a First Extrachromosomal Plasmid with a Copy of the ziaRA Genes and Ethanologenic Genes

FIG. 17C shows the ethanol production rates of three ethanologenic Synechocystis sp. PCC 6803 strains denoted #864_{—}2, #864_{—}4, #864_{—}17 including a first extrachromosomal plasmid #864 which is a pVZ plasmid including a ziaRA genes and again Pdc encoding gene under the control of the petJ promoter and Synechocystis Adh encoding gene SynAdh_{deg} under the control of the rbcL promoter. The graph denoted #550 shows the ethanol production rate of a conventional cyanobacterial strain harbouring a plasmid #550 conferring Gentamycin resistance. It is again clearly visible that the genetically enhanced cyanobacterial strains lacking the biocide conferring resistance gene show similar ethanol production rates to conventional cyanobacterial strains. The pre-cultures for this experiment contained 5 μM Zn²⁺ in the BG11 growth medium.

V. Construction of an Antibiotic-Resistance-Cassette Free Expression System Based on the narB Gene Inactivation and Including a First Extrachromosomal Plasmid with the narB Gene and a First Production Gene for Ethylene Formation in Synechocystis sp.PCC 6803

FIG. 18A shows the general features of a genetically enhanced cyanobacterium harboring a genomic first gene inactivation in the narB gene and also including a first extrachromosomal plasmid including a narB gene and a gene encoding an ethylene forming enzyme (efe); The below graph shows the ethylene production rate of these genetically enhanced cyanobacteria. Ethylene production was determined by online GC measurements using illuminated cultures in GC vials. Online GC measurements were carried out as mentioned above. The graphs denoted narB_reference_{—}1 and narB_reference_{—}2 show that cyanobacteria harbouring a first gene inactivation in the narB gene, but lacking efe as a production gene do not produce ethylene. The sequences denoted “P1 narB” and “P2 narB” mark 5′ and 3′ nucleic acid sequences, which are present in the ΔnarB locus of Synechocystis sp. PCC 6803 strains harbouring a first gene inactivation in the narB gene done via deletion. These P1 and P2 platforms were introduced via homologous recombination through the second recombinant nucleic acid into the cyanobacteria and replaced the first recombinant nucleic acid sequence.

VI. Construction of an Antibiotic-Resistance-Cassette Free Expression System Based on the ziaRA Genes Inactivation and Including a First Extrachromosomal Plasmid with the ziaRA Genes and a First Production Gene for Ethylene Formation in Synechocystis Sp.PCC 6803

FIG. 18B shows the general features of a genetically enhanced cyanobacterium harbouring a first genomic gene inactivation in the ziaRA genes and also including a first extrachromosomal plasmid including the ziaRA genes and a gene encoding an ethylene forming enzyme; The below graph shows the ethylene production rate of these genetically enhanced cyanobacteria. Ethylene production was determined by online GC measurements using illuminated cultures in GC vials. The graphs denoted ziaRA_reference_{—}1 and ziaRA_reference_{—}2 show that cyanobacteria harbouring a first gene inactivation in the ziaRA gene, but lacking efe as a production gene do not produce ethylene.

VII: Construction of an Antibiotic-Resistance-Cassette Free Expression System Based on Co²⁺ Sensitive Phenotype Via corRT Gene Inactivation for Generation of Ethanologenic Hybrids in Synechocystis Sp.PCC 6803

A Synechocystis sp.PCC 6803 genetically enhanced cyanobacterial cell was created harboring a first gene inactivation in the first conditionally essential corRT genes by a complete deletion of the corRT genes, leading to sensitivity against Co²⁺ and including a first extrachromosomal plasmid harboring first production genes for production of ethanol and additionally also the corRT genes to complement for the first gene inactivation. These cyanobacterial cells with a Co²⁺ sensitive phenotype were created in the same way as the below in the experimental protocol section described cyanobacterial cells including a leuB gene inactivation and a ziaRA gene inactivation. In particular, plasmids #818 and #822 were used in order to first replace the corRT genes with a SacB/Gm cassette and secondly to replace this cassette with a ΔcorRT sequence via homologous recombination using the homologous platforms P1 corT and P2 corT included in both plasmids. These cells can be cultivated in BG11 medium containing higher amounts of Co²⁺ such as 5 μM.

FIG. 23A shows a DNA agarose gel evidencing complete segregation of the corRT gene inactivated cyanobacterial cells which exhibit a Co²⁺ sensitive phenotype. The lane denoted “6803 WT control” shows the signal of the wild type corRT gene locus which is not existent in any of the colonies “cl.8”, “cl.22” and “cl.25” isolated from the gene inactivation procedure after method step i4) of the method for producing the cyanobacterial cells. In these colonies the amplified ΔcorRT locus has a smaller size than the ΔcorRT(SacB/Gm) locus or the 6803 wild type allele of the corRT locus due to the first gene inactivation, which involves a complete deletion of the corRT gene. These colonies “cl.8”, “cl.22” and “cl.25” do not show signals for the ΔcorRT(SacB/Gm) locus or the 6803 wild type allele.

The DNA agarose gel in FIG. 23B shows that the first extrachromosomal plasmids #861 or #870 comprising PDC and synAdh gene are present in the tested colonies after method step ii) of the method for producing the cyanobacterial cells. These first extrachromosomal plasmids also contain the corRT genes (including the native promoters PcorR and PcorT) for restoration of the cobalt tolerance. The plasmid #861 contains the sequence pVZhisB-corRT-PpetJ-PDC-Prbc*-synADHdeg and #870 the sequence pVZhisB-corRT-PpetJ-PDCoop-Prbc*-synADH_{deg}. These plasmids include the ethanologenic genes coding for PDC and SynAdh_{deg} along with the hisB gene for selection in E. coli KC8 strain (histidine auxotroph) and the corRT genes. In contrast to plasmid #861 in plasmid #870 the termination of transcription of the PDC gene is achieved by addition of the oop-terminator of the lambda phage at the 3′-end of the PDC gene (PDCoop).

FIG. 23C shows a PCR control specific for E. coli cells. This is to exclude any E. coli leftovers harboring #861 or #870 from conjugation process which would result in a false positive signal for #861 or #870 plasmids shown in the middle panel.

The graph in FIG. 23D shows stable ethanol production rates in GC vial assay for Synechocystis ΔcorRT strains complemented with the corRT-ethanologenic plasmids #861 or #870 in comparison to ethanologenic Synechocystis strains carrying a conventional plasmid with an antibiotic resistance cassette (ABR) cassette “ABR reference strain for plasmid #861 and #870”.

Genetically enhanced Synechocystis strains were cultivated on BG11 plates with either 5 μM cobalt or gentamycin to maintain the first extrachromosomal plasmids. In order to induce the ethanol production the cells were grown under copper depleted conditions (BG11 —Cu plates) 3 days prior the GC vial measurement (transcriptional control of the ethanologeneic genes by the petJ promoter). For the GC vial assay cells were scratched from BG11 plates and subsequently cultivated in 2 ml GC vials containing copper-free mBG11 growth medium (marine BG11 medium) in the light. The ethanol production rate for both ΔcorRT clones #870.1 and #870.2 is higher than for strains containing plasmid #861. This is due to the added oop terminator sequence encoded on plasmid #870(PpetJ-PDCoop-Prbc*-synADH_{deg})

FIG. 23E shows the ethanol production and FIG. 23F the ethanol production normalized on OD₇₅₀ₙₘ of three genetically enhanced Synechocystis strains over a time frame of 27 days. The strains were cultivated in 0.5 L photobioreactors with mBG11 growth medium supplemented with either 5 μM Co²⁺, 10 μM Zn²⁺ or gentamycin to maintain the ethanologenic plasmids. In the shown experiment two ABR free ethanol producers, ΔziaRA with plasmid #1359 (pVZhisB-PhspA-ziaA(6803)-PpetJ-PDCdsrA-Prbc*-synADH_{deg}) and ΔcorRT with plasmid #870 (pVZhisB-corRT-PpetJ-PDCoop-Prbc*-synADH_{deg}) were compared to a conventional ABR containing ethanol producer with plasmid #990 (pVZ322a-PpetJ-PDCoop-PrbcL-synADH_{deg} Km^{R}/Gm^{R}). “dsrA” denotes a terminator sequence derived from the small non-coding RNA DsrA from E. coli [Lesnik et al.” “Prediction of Rho independent terminators in E.coli”, Nucl. Acids Res. (2001) 29 (17): 3583-3594]. The outcome is a stable ethanol production rate from 0.024%(v/v)d⁻¹ to 0.032%(v/v)d⁻¹ for all three strains over the entire time of cultivation. The slight differences in the total ethanol production rate is a result of the slightly different ethanologenic gene cassettes.

VIII: Construction of an Antibiotic-Resistance-Cassette Free Expression Double Knock Out System Based on a Zn²⁺ Sensitive Phenotype Via ziaRA Gene Inactivation and Lack of Ability to Use Nitrate as a Nitrogen Source Via narB Gene Inactivation for Generation of Ethanologenic Hybrid Strains in Synechocystis Sp.PCC 6803

Via the methods described below in the experimental protocols, genetically enhanced cyanobacterial strains can be produced which include a first gene inactivation in the narB gene or apart from this inactivation also a second gene inactivation in the ziaRA gene (double knock out).

In FIGS. 24A and 24B two DNA agarose gels are presented showing complete segregated double knock-out cyanobacterial cells “cl.1” and “cl.2” of ziaRA gene as a first gene inactivation and narB gene as a second gene inactivation after the method step i4), the selection for transformed cyanobacteria from step i3) via subjecting the cyanobacteria to the counterselectable sucrose marker. The lanes denoted “Syn. 6803 WT” in both figures exhibit the signal of the wild type ziaRA gene and narB gene which are not existent in the clones “cl.1” and “cl.2”. In these clones the amplified ΔziaRA and ΔnarB loci are of smaller size than the respective ΔziaRA(SacB/Gm) and ΔnarB(SacB/Gm) loci or the 6803 wild type allele due to the first gene inactivation, which involves a complete deletion of the ziaRA and narB genes. The double mutants ΔziaRA/ΔnarB “cl.1” and “cl.2” are unable to use nitrate as a sole nitrogen source (which is included in normal BG11 medium) and can't tolerate >3 μM Zn²⁺ in the growth medium.

The DNA agarose gels in FIG. 24C show the results of a primer specific PCR for two successfully complemented ΔnarB/ΔziaRA clones “cl.1” and “cl.2” by co-conjugation with the ethanologenic pVZ-narB plasmid #820 and the pVZ-ziaRA plasmid #864 as first and second extrachromosomal plasmids after method step ii). The ethanologenic plasmid #864 contains the sequence pVZhisB-ziaRA-PpetJ-PDC-Prbc*-synADH_{deg} and #820 the sequence pVZhisB-PnirA*-narB-PpetJ-PDC/synADH. The used primer pairs are specific for the different synADH genes present in both plasmids.

The lower panel of FIG. 24C shows a PCR control specific for E. coli cells. This is to exclude any E. coli leftovers harboring #864 or #820 from conjugation process which would result in a false positive signal for synADH and synADH_{deg} specific PCR.

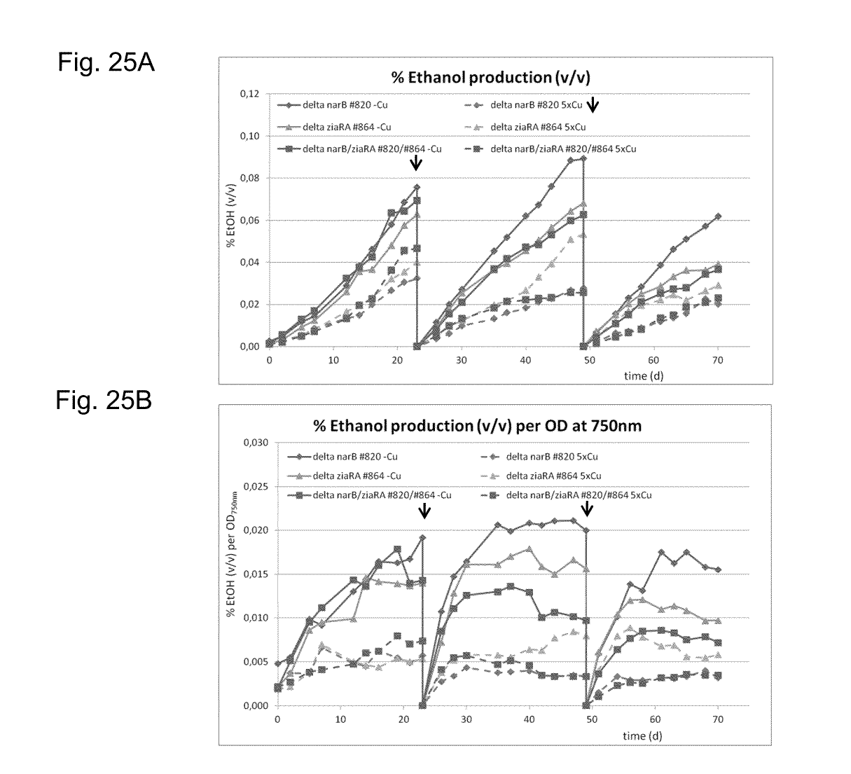

In FIG. 25A the ethanol production and in FIG. 25B the ethanol production normalized on OD₇₅₀ₙₘ of the antibiotic resistance free cyanobacterial strains ΔnarB #820, ΔziaRA #864 and the double variant ΔnarB/ΔziaRA containing both plasmids #820/#864 are presented. This long term experiment was running over a time frame of about 70 days in 50 ml Erlenmeyer flasks. For plasmid maintenance ΔnarB #820 was grown in normal mBG11 medium (NO₃ is sole nitrogen source) whereas ΔziaRA #864 and the double variant ΔnarB/ΔziaRA that contains both plasmids #820/#864 were cultivated in mBG11 supplemented with 5 μM Zn²⁺. The expression of the ethanologenic genes is regulated via the copper-deficiency inducible petJ promoter. In the absence of copper in mBG11 medium all three ABR free strains show stable ethanol production rates, also after every medium exchange (indicated by red arrows). These results show that the cyanobacteria maintain the ABR free first and second extra-chromosomal plasmids within the cells over the entire time of the cultivation experiment without any use of conventional antibiotic selection systems.

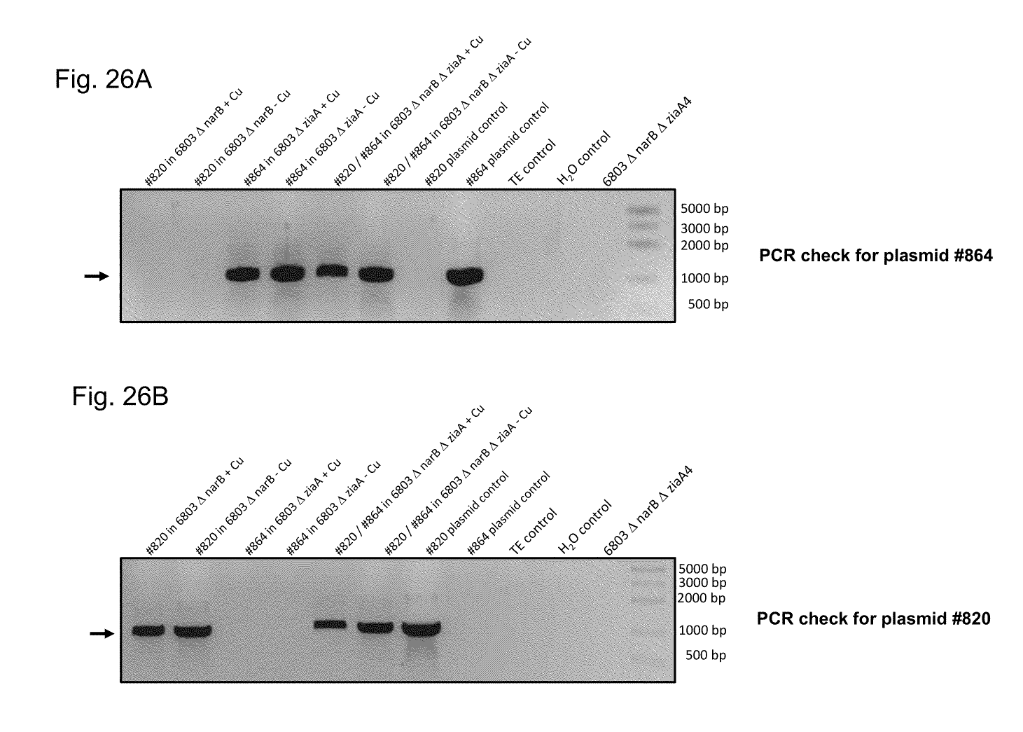

In order to confirm the stable maintenance of the ethanologenic plasmids the presence of plasmid #820 and #864 was verified at the end of the cultivation experiment by a plasmid specific PCR. In FIG. 26A and FIG. 26B two DNA agarose gels are presented showing PCR products specific for plasmid #820 in ΔnarB and in ΔnarB/ΔziaRA double knockout variant whereas the PCR product specific for plasmid #864 is present in ΔziaRA as well as the double knockout variant. Therefore the presence of both ethanologenic plasmids could be detected in the Synechocystis ΔnarB/ΔziaRA strain demonstrating a stable replication of two co-existing plasmids with the same replicon by application of an ABR-free double selection system based on narB and ziaRA.

IX: Construction of an Antibiotic-Resistance-Cassette Free Expression System Based on a Zn²⁺ Sensitive Phenotype Via smtAB Gene Inactivation Including a First Extrachromosomal Plasmid Harboring ziaA from Synechocystis PCC6803 and Ethanologenic Genes in Synechococcus PCC7002

FIG. 30A shows a DNA agarose gel with four completely segregated ΔsmtAB clones (“cl.1-4”) in Synechococcus PCC7002. The “PCC7002 WT control” shows the signal of the wild type smtAB genes which are not existent in any of the colonies “cl. 1-4” from the gene inactivation procedure. In these colonies the amplified ΔsmtAB locus has a smaller size than the ΔsmtAB(SacB/Gm) locus or the wild type allele of the smtAB locus due to the first gene inactivation, which involves a complete deletion of the smtAB genes and a zinc sensitive phenotype as consequence. The first gene inactivation is achieved via homologous recombination with the linearized plasmid #1160 which includes the two platforms smtA/B P1 and smtA/B P2 for integration into the chromosome via homologous recombination and the SacB/Gm cassette replacing the endogenous smtAB genes. This SacB/Gm cassette is subsequently replaced by the ΔsmtAB sequence using the linearized plasmid #1228, which only includes the two platforms smtA/B P1 and smtA/B P2 for integration into the chromosome via homologous recombination.

The DNA agarose gel in FIG. 30B shows the result of a primer specific PCR of ΔsmtAB clones (“cl.1, 4, 7, 9”) in Synechococcus PCC7002 for the first extra-chromosomal plasmid #1326 encoding Pdc and SynAdh enzyme. This first extra-chromosomal plasmid also contains the ziaA gene, a functional analogue of smtA from Synechocystis PCC6803, under control of the hspA promoter for restoration of the zinc tolerance in the Synechococcus ΔsmtAB cell line. The extra-chromosomal plasmid #1326 contains the sequence pVZhisB-PhspA-ziaA-P_{ind}-PDCdsrA-Prbc*-synADH_{deg}. The term “P_{ind}” denotes a promoter, which is inducible in Synechococcus such as the nirA promoter [Maeda et al., 1998].

FIG. 30C shows a PCR control specific for E. coli cells. This is to exclude any E. coli leftovers harboring #1326 from conjugation process of Synechococcus ΔsmtAB cell line which would result in a false positive signal for plasmid #1326 in the middle panel.

The graph in FIG. 31A shows stable ethanol production rates in GC vial assay for Synechococcus PCC7002 ΔsmtAB cell line complemented with the ziaA containing ethanologenic first extrachromosomal plasmid #1326 (pVZhisB-PhspA-ziaA-P_{ind}-PDCdsrA-Prbc*-synADH_{deg}) in comparison to an ethanologenic Synechococcus PCC7002 strain carrying a conventional plasmid with an ABR cassette “ABR containing reference”. Both Synechococcus hybrid strains were cultivated on BG11 plates with either 5 μM zinc or gentamycin to assure plasmid maintenance. 2 days prior to the GC vial assay cells were transferred to specific BG11 plates for pre-induction. For the GC vial assay cells were scratched from these plates and cultivated subsequently in 2 ml GC vials in the light containing mBG11 growth medium. As evident the ethanol production rate for both ΔsmtAB clones #1326.1 and #1326.2 is similar to the ABR containing reference strain.

In FIG. 31B the ethanol production and in FIG. 31C the ethanol production normalized on OD at 750 nm of a Synechococcus PCC7002 ΔsmtAB strain complemented with the ziaA containing extrachromosomal ethanologenic plasmid #1326 in comparison to a wild type with conventional ethanologenic plasmid “ABR reference” is shown. Both were grown in 0.25 L Schott flasks with mBG11 medium aerated with CO₂ enriched air with (5% CO₂). The medium for ΔsmtAB #1326 was supplemented with 5 μM zinc whereas the ethanologenic WT reference was supplemented with kanamycin to assure plasmid maintenance. Plasmid #1326 is a self-replicating pVZ plasmid comprising the ziaA gene under the transcriptional control of the hspA promoter both derived from Synechocystis PCC6803. The presented ethanol production rates for both genetically enhanced cyanobacteria are similar over the analyzed period of time, which demonstrates there is no disadvantage when the ethanologenic plasmid contains an ABR free selection system in comparison to a conventional antibiotic resistance cassette (Gm^{R}/Km^{R}).

These ethanol production rates clearly show that a first gene inactivation in conditionally essential genes such as smtAB can be complemented by the introduction of a first extrachromosomal plasmid harbouring genes analogous to the inactivated conditionally essential genes such as ziaA. The first extrachromosomal plasmid also contains first production genes for ethanol production.

X: Construction of an Antibiotic-Resistance-Cassette Free Expression System Based on a Zn²⁺ Sensitive Phenotype Via smtAB Gene Inactivation Including a First Extrachromosomal Plasmid Harboring Endogenous smtAB and Ethanologenic Genes in Synechococcus PCC7002

FIG. 33A shows a DNA agarose gel with nine completely segregated ΔsmtAB clones (“cl.1-9”) in Synechococcus PCC7002 complemented with the extra-chromosomal ethanologenic plasmid #1454 which comprises the smtAB genes from Synechococcus PCC7003 (including the native promoters PsmtB and PsmtA) for restoration of the zinc tolerance. The “PCC7002 WT control” shows the signal of the wild type smtAB genes which are not existent in any of the colonies “cl. 1-9” from the gene inactivation procedure. In these colonies the amplified ΔsmtAB locus has a smaller size than the ΔsmtAB(SacB/Gm) locus or the wild type allele of the smtAB locus which confirms a successful deletion of the smtAB genes in the PCC7003 chromosome.

The DNA agarose gel in FIG. 33B shows the result of a primer specific PCR of the same ΔsmtAB clones (“cl.1-9”) in Synechococcus PCC7002 for the first endogenous plasmid #1454 encoding Pdc and SynAdh enzyme. This first endogenous plasmid also contains the endogenous smtAB genes from Synechococcus PCC7002 for restoration of the inactivated zinc tolerance in the Synechococcus ΔsmtAB cell line. The extra-chromosomal self-replicating plasmid #1454 contains the sequence pVZhisB-smtAB-Pind-PDCdsrA-Prbc*-synADH_{deg}.

FIG. 33C shows a PCR control specific for E. coli cells. This is to exclude any E. coli leftovers harboring #1454 from conjugation process of Synechococcus ΔsmtAB cell line which would result in a false positive signal for plasmid #1454 in the middle panel.

The graph in FIG. 33D shows stable ethanol production rates in GC vial assay Synechococcus PCC7002 ΔsmtAB for three independent cell line complemented with the extra-chromosomal ethanologenic plasmid #1454 (pVZhisB-smtAB-Pind-PDCdsrA-Prbc*-synADH_{deg}) in comparison to an ethanologenic Synechococcus PCC7002 reference strain carrying a conventional plasmid with an ABR cassette “ABR containing reference”. Both Synechococcus hybrid strains were cultivated on BG11 plates with either 5 μM zinc or gentamycin to assure plasmid maintenance. For the GC vial assay uninduced cells were scratched from BG11 plates and cultivated subsequently in the light under induced conditions in 2 ml GC vials containing mBG11 growth medium. As evident the ethanol production rate for all three ΔsmtAB clones #1454.1, #1454.2 and #1454.3 is very similar to the ABR containing PCC7002 reference strain.

XI: Construction of an Antibiotic-Resistance-Cassette Free Expression System Based on a Zn²⁺ Sensitive Phenotype Via smtAB Gene Inactivation Including a First Extrachromosomal Plasmid Based on the Endogenous Synechococcus pAQ3 Plasmid, Harboring ziaA Gene and Ethanologenic Genes in Synechococcus PCC7002

FIG. 35A shows a DNA agarose gel with three completely segregated ΔsmtAB clones (“cl.1-6”) in Synechococcus PCC7002 transformed with the pAQ3-integrative ethanologenic plasmid #1484 which comprises the ziaA gene from Synechocystis under control of the hspA promoter for restoration of the zinc tolerance and which can integrate into the endogenous pAQ3 plasmid via homologous recombination. The “PCC7002 WT control” shows the signal of the wild type smtAB genes which are not existent in any of the colonies “cl. 1-6” from the gene inactivation procedure. In these colonies the amplified ΔsmtAB locus has a smaller size than the ΔsmtAB(SacB/Gm) locus or the wild type allele of the smtAB locus which confirms a successful deletion of the smtAB genes in the PCC7003 chromosome.

The DNA agarose gel in FIG. 35B shows the result of a primer specific PCR of the same ΔsmtAB clones (“cl.1-3”) in Synechococcus PCC7002 for the first endogenous plasmid #1484 encoding Pdc and SynAdh enzyme. This first endogenous plasmid also contains the ziaA gene from Synechocystis PCC6803 for restoration of the zinc tolerance in the Synechococcus ΔsmtAB cell line. The suicide plasmid #1484 for integration into the endogenous pAQ3 plasmid via homologous recombination contains the sequence pAQ3::PhspA-ziaA-P_{ind}-PDCdsrA-Prbc*-synADH_{deg}.

The graph in FIG. 35C shows stable ethanol production rates in GC vial assay for Synechococcus PCC7002 ΔsmtAB cell line complemented with the pAQ3-integrative ethanologenic plasmid #1484 which comprise the ziaA gene from Synechocystis for restoration of the zinc tolerance (pAQ3::PhspA-ziaA-P_{ind}-PDCdsrA-Prbc*-synADH_{deg}). The Synechococcus hybrid strains were cultivated on BG11 plates with 5 μM zinc to assure plasmid maintenance. For the GC vial assay uninduced cells were scratched from plates and cultivated subsequently in the light under repressed vs. induced conditions in 2 ml GC vials containing mBG11 growth medium. As evident the ethanol production rate for all three ΔsmtAB clones #1484.1, #1484.2 and #1326.3 is very similar at induced conditions.

XII: Construction of an Antibiotic-Resistance-Cassette Free Expression System Based on a Zn²⁺ Sensitive Phenotype Via smtAB Gene Inactivation Including a First Extrachromosomal Plasmid Based on the Endogenous Synechococcus pAQ4 Plasmid, Harboring ziaA Gene and Ethanologenic Genes in Synechococcus PCC7002

FIG. 37A shows a DNA agarose gel with a completely segregated ΔsmtAB clone in Synechococcus PCC7002 transformed with the pAQ4-integrative ethanologenic plasmid #1489 which comprises the ziaA gene from Synechocystis for restoration of the zinc tolerance. The “PCC7002 WT control” shows the signal of the wild type smtAB genes which is not existent in the analyzed clone from the gene inactivation procedure. In this clone the amplified ΔsmtAB locus has a smaller size than the ΔsmtAB(SacB/Gm) locus or the wild type allele of the smtAB locus which confirms a successful deletion of the smtAB genes in the PCC7003 chromosome.

The DNA agarose gel in FIG. 37B shows the result of a primer specific PCR of the same ΔsmtAB clone in Synechococcus PCC7002 for the first endogenous plasmid #1489 encoding Pdc and SynAdh enzyme. This first endogenous plasmid contains beside the ethanologenic genes the ziaA gene from Synechocystis PCC6803 under control of the hspA promoter for restoration of the zinc tolerance in the Synechococcus ΔsmtAB cell line. The suicide plasmid #1489 for integration into the endogenous pAQ4 plasmid contains the sequence pAQ4::PhspA-ziaA-Pind-PDCdsrA-Prbc*-synADH_{deg}.

The graph in FIG. 37C shows a stable ethanol production rate in GC vial assay for Synechococcus PCC7002 ΔsmtAB cell line complemented with the pAQ4-integrative ethanologenic plasmid #1489 which comprise the ziaA gene from Synechocystis for restoration of the zinc tolerance (pAQ4::PhspA-ziaA-Pind-PDCdsrA-Prbc*-synADH_{deg}). The Synechococcus hybrid strain was cultivated on a BG11 plate with 7.5 μM zinc to assure plasmid maintenance. For the GC vial assay uninduced cells were scratched from BG11 plates and cultivated subsequently in the light under repressed, moderate induced and fully induced conditions in 2 ml GC vials containing mBG11 growth medium. As evident the ethanol production rates for the ABR-free hybrid cell line ΔsmtAB #1489 is strongly dependent from the induction conditions and in a similar range as frequently measured for an conventional ABR containing ethanologenic Synechococcus PCC7002 strain at the induced condition.

FIG. 38 schematically shows several metabolic pathways in cyanobacterial cells. In particular, the glycolysis pathway leading from 3PGA to pyruvate is shown, the citric acid cycle and the Calvin cycle as well as the pentose phosphate pathway. The enzymes marked with a squared box are prime candidates for overexpression in cyanobacterial cells, which harbour a first extrachromosomal plasmid including first production genes encoding ethanologenic enzymes for the production of ethanol. In this case a second extrachromosomal plasmid can be present in the cyanobacterial cells, which includes a second production gene encoding any of the enzymes marked with rectangular boxes.

### Experimental Protocols

Recipe for BG11 growth medium for cyanobacteria:
NaNO₃: 1.5 g
K₂HPO₄: 0.04 g
MgSO₄.7H₂O: 0.075 g
CaCl₂.2H₂O: 0.036 g
Citric acid: 0.006 g
Ferric ammonium citrate: 0.006 g
EDTA (disodium salt): 0.001 g
NaCO₃: 0.02 g
Trace metal mix A5_{—}1.0 ml
Agar (if needed): 10.0 g
Distilled water: 1.0 L
The pH should be 7.1 after sterilization
Trace metal mix A5:
H₃BO₃: 2.86 g
MnCl₂.4H₂O: 1.81 g
ZnSO₄.7H₂O: 0.222 g
NaMo0₄.2H₂O: 0.39 g
CuSO₄.5H₂O: 0.079 g
Co(NO₃)₂.6H₂O: 49.4 mg
Distilled water: 1.0 L.

Marine BG11 medium (mBG11) can be produced by using seawater (35 practical salinity units=psu; see Unesco (1981a). The Practical Salinity Scale 1978 and the International Equation of State of Seawater 1980. Tech. Pap. Mar. Sci., 36: 25 pp.) instead of distilled water.

The following experimental protocols can be employed for the generation of the genetically enhanced cyanobacterial cells of the invention I including a first gene inactivation not just in the leuB or ziaRA genes, but also in other essential or conditionally essential genes.

A: Construction of an Antibiotic-Resistance-Cassette Free Expression System Based on the Zinc Tolerance Via ziaRA for Generation of Genetically Enhanced Ethanologenic Synechocystis Sp.PCC 6803 Cyanobacteria

This experimental protocol employs the above described method steps i1) to i4) for production of a genetically enhanced Synechocystis sp. PCC 6803 cyanobacterium harboring a first gene inactivation in the conditionally essential ziaRA genes, followed by method step ii) of introducing the first extrachromosomal plasmid into the cyanobacterium including ethanologenic Pdc and Adh encoding genes and also the genes encoding ziaRA. During method steps i1) to i4), the growth media of the cyanobacteria only contained Zn²⁺ in concentrations of less than 2 μm, in order to ensure that the genetically enhanced cyanobacteria harboring the first gene inactivation are not killed by high Zn²⁺ concentrations. This can be done by using normal BG11 growth medium, which contains less than 0.77 μm of Zn²⁺.

A.1) SacB/Gm Mediated Gene Inactivation of ziaRA

The sacB gene encodes the enzyme levansucrase from Bacillus subtilis that confers sucrose sensitivity on gram-negative bacteria. The sacB gene was site-directed (via platforms P1/P2 for homologous recombination) introduced into the cyanobacterial genome along with a Gentamycin resistance cassette to replace the ziaRA. P1 and P2 again denote 1200 bp neighboring 5′ and 3′ nucleic acid sequences to the wild type ziaRA genes, which enable homologous recombination due to sequence identity with the first and second recombinant nucleic acids.

Procedure: Transformation of pJet-ziaRA P1/P2-sacB/Gm construct in Synechocystis sp. PCC 6803 by natural competence
1. Cells of Synechocystis sp. PCC 6803 were cultivated to mid-log phase (OD₇₅₀≈1) on a rotary shaker at 28° C.
2. 12 ml of the culture were centrifuged at room temperature (10 min, 4.000 rpm in Hettich Rotina 240R Falcon tube centrifuge).
3. The supernatant was decanted and the pellet resuspended in the remaining medium (200 μl).
4. After transferring the 200 μl into a 1.5 ml Eppendorf tube, 0.5 μg of DNA (pJet-ziaRA P1/P2-sacB/Gm) were added. It followed an incubation time for at least 1 hour at room temperature, inverting the tube in between.
5. The suspension was plated on a BG11-1% agar plate without antibiotics and incubated at 28° C. under low light (25-35 μmol/m²*sec).
6. After 2 days 300 μl Gentamycin (1 mg/ml) were placed at one site of the agar plate to form a gradient of concentration within the plate as a result of diffusion.
7. Most of the biomass died and turned yellow. After 5 weeks single transformed blue-green colonies appeared. These colonies were picked and transferred to new plates with 2 μg/ml gentamycin. Then three passages on 5 μg/ml Gentamycin plates followed for full segregation. The whole procedure took about 2 month. The segregation status was checked by PCR.
   A.2) Sucrose Mediated Removal of sacB/Gm Cassette from ΔziaRA

In a second step via the same P1/P2 recombination platforms the sacB/Gm cassette was replaced. This step is driven by the loss of the sucrose sensitivity.

Procedure: Transformation of pJet-ziaRA P1/P2 construct (without sacB/Gm cassette) in the ΔziaRA(sacB/Gm) strain (result of previous step) by natural competence
1. Cells of ΔziaRA(sacB/Gm) were cultivated to mid-log phase (OD₇₅₀≈1) on a rotary shaker at 28° C.
2. 12 ml of the culture were centrifuged at room temperature (10 min, 4.000 rpm in Hettich Rotina 240R Falcon tube centrifuge).
3. The supernatant was decanted and the pellet resuspended in the remaining medium (200 μl).

4. After transferring the 200 μl into a 1.5 ml Eppendorf tube, 0.5 μg of DNA (pJet-ziaRA P1/P2) were added. It followed an incubation time for at least 1 hour at room temperature, inverting the tube in between.
5. The suspension was plated on a HATF (nitrocellulose membrane) filter (e.g. Millipore Durapore, 0.22 μm) which was located on top of a BG11-1% agar plates without antibiotics. The cells were incubated at 28° C. under low light (25-35 μmol/m²*sec).
6. After 2 days the filter was transferred on a BG11 plate supplemented with 2% sucrose and additional two 2 days later on a BG11 plate with 5% sucrose.
7. Most of the biomass on the filter died but single transformed blue-green colonies appeared within 15 days. These were picked and transferred to new BG11 plates with 5% sucrose.
8. To select the ΔziaRA clones from potential false positive clones with only sucrose resistance a test was performed comparing the same colonies on BG11+5% sucrose and on BG11+5 μg/ml Gentamycin plates. Intended clones should die in the presence of gentamycin. To exclude wildtype revertants a PCR control was done.
   A.3) Complementation of ABR-Free, Zn²⁺ Sensitive Synechocystis ΔziaRA Strain with Ethanologenic pVZ-ziaRA Plasmid Via Conjugation

Procedure: Conjugation of pVZ-hisB-ziaRA-PpetJ-PDC-PrbcLS*-synADH_{deg} construct in Synechocystis ΔziaRA strain by tri-parental mating
1.3 ml overnight culture with E. coli J53[RP4] (50 μg/ml ampicillin and 20 μg/ml kanamycin) and 3 ml overnight culture with E. coli pVZ-hisB-ziaRA-PpetJ-PDC-PrbcL*-synADH_{deg} were inoculated.
2.250 μl of the E. coli overnight cultures were transferred in 10 ml fresh LB medium each, the E. coli J53[RP4] with appropriate antibiotics, and cultured in 100 ml Erlenmeyer flasks for 2.5 h shaking at 37° C.
3. Then the cells were sedimented by 8 min centrifugation in 15 ml falcon tubes at 2500 rpm and room temperature in a Hettich Rotina 240R centrifuge.
4. Both pellets were resuspended and mixed together in 2 ml LB medium in a 2 ml Eppendorf tubes and centrifuged again (5 min, 2500 rpm in Hettich Mirco 200R centrifuge).
5. The combined pellet was resuspended in 100 μl LB medium and incubated without shaking 1 h at 30° C.
6. Then 1.9 ml Synechocystis ΔziaRA culture with OD₇₅₀≈0.8 (mid-log phase) was added. The culture was slightly shaken and centrifuged (5 min, 2500 rpm in Hettich Mirco 200R centrifuge).
7. The pellet was resuspended in 50 μl BG-11 and dropped on a HATF (nitrocellulose membrane) filter (e.g. Millipore Durapore, 0.22 μm) which was located on top of a prepared plate (40 ml Bg11-agar+2 ml LB medium without antibiotics).
8. The plate was incubated for 2 days under low light conditions (25-35 μmol/m²*sec) at 28° C.
9. After incubation the bacteria were splashed of the filter with 200 μl BG-11, 1:10 diluted and carefully plated on 1%-agar plates with 2 μM, 5 μM and 10 μM ZnSO₄. Selection took place via restoration of the Zn²⁺ tolerance.
10. After 7 days first transconjugants were visible at all Zn²⁺ concentrations (2-15 μM ZnSO₄). The control conjugation assay using only E. coli J53[RP4] and no E. coli pVZ-hisB-ziaRA-PpetJ-PDC-PrbcL*-synADH_{deg} didn't show any colonies on the BG11-agar plates with different ZnSO₄ concentrations. Transconjugants were verified by PCR.
   B: Construction of an Antibiotic-Resistance-Cassette Free Expression System Based on the Leucine Auxotrophy Via leuB Gene Inactivation for Generation of Ethanologenic Hybrids in Synechocystis Sp.PCC 6803

This experimental protocol for introducing a gene inactivation into the first essential leuB gene employs the above described method steps i′1) to i′2) at the beginning for introducing a first gene inactivation in not all genomic copies of the leuB gene. Subsequently, the method step ii) for introducing the first extrachromosomal plasmid is carried out in order to complement for the first gene inactivation. This first extrachromosomal plasmid includes ethanologenic Pdc and Adh encoding genes and also a gene encoding leuB. This method step ii) is followed by the above described method steps i′3) to i′5) in order to finally produce the genetically enhanced cyanobacterium harboring the first gene inactivation in all genomic copies of the leuB gene.

B. 1) SacB/Cm Mediated Gene Inactivation of leuB

The sacB gene encodes the enzyme levansucrase from Bacillus subtilis that confers sucrose sensitivity on gram-negative bacteria. The sacB gene was site-directed (via 740 bp P1 and P2-platforms for homologous recombination) introduced into the cyanobacterial genome along with a Chloramphenicol resistance cassette to delete the leuB gene.

Procedure: Transformation of pJet-leuB P1/P2-sacB/Cm construct in Synechocystis sp. PCC 6803 by natural competence
1. Cells of Synechocystis sp. PCC 6803 were cultivated to mid-log phase (OD₇₅₀≈1) on a rotary shaker at 28° C.
2. 12 ml of the culture were centrifuged at room temperature (10 min, 4.000 rpm in Hettich Rotina 240R Falcon tube centrifuge).
3. The supernatant was decanted and the pellet resuspended in the remaining medium (200 μl).
4. After transferring the 200 μl into a 1.5 ml Eppendorf tube, 0.5 μg of DNA (pJet-leuB P1/P2-sacB/Cm) were added. It followed an incubation time for at least 1 hour at room temperature, inverting the tube in between.
5. The suspension was plated on a BG11-1% agar plate without antibiotics and incubated at 28° C. under low light (25-35 μmol/m²*sec).
6. After 2 days 400 μl Chloramphenicol (4 mg/ml) were placed at one site of the agar plate to form a gradient of concentration within the plate as a result of diffusion.
7. Most of the biomass died and turned yellow. After 4 weeks single transformed blue-green colonies appeared. These colonies were picked and transferred to new plates with 2 μg/ml Chloramphenicol and 200 μg/ml leucine. Then three passages on plates supplemented with 200 μg/ml leucine and 5 μg/ml, 10 μg/ml and 30 μg/ml Chloramphenicol, respectively followed in order to reach complete segregation. The whole procedure took about 3 month. The segregation status was checked by PCR.

In spite of the presence of leucine within the plates the complete segregation was not accomplished. Despite repeated restreaks on plates supplemented with 100 μg/ml Chloramphenicol and 400 μg/ml leucine it was not possible to reach a complete segregation of the leuB gene inactivation. For the removal of the sacB/Cm cassette by sucrose selection (second step) a complete segregation of the leuB disruption is imperative. Therefore the actual third step, the complementation of the leuB gene inactivation by introduction of a second leuB copy encoded on the extrachromosomal pVZ plasmid was pull out from behind.

B.2) Complementation of Incomplete Segregated Synechocystis ΔleuB(sacB/Cm) Strain with Ethanologenic pVZleuB₇₁₂₀ Plasmid Via Conjugation

Procedure: Conjugation of pVZ-leuB₇₁₂₀-PpetJ-PDC/SynADH construct in ΔleuB(sacB/Cm) strain by tri-parental mating
1.3 ml overnight culture with E. coli J53[RP4] (50 μg/ml ampicillin and 20 μg/ml kanamycin) and 3 ml overnight culture with E. pVZ-leuB-PpetJ-PDC/SynADH were inoculated.
2.250 μl of the overnight E. coli cultures were transferred in 10 ml fresh LB medium each, the E. coli J53[RP4] with appropriate antibiotics, and cultured in 100 ml Erlenmeyer flasks for 2.5 h shaking at 37° C.
3. Then the cells were sedimented by 8 min centrifugation in 15 ml falcon tubes at 2500 rpm and room temperature in a Hettich Rotina 240R centrifuge.
4. Both pellets were resuspended and mixed together in 2 ml LB medium, in a 2 ml Eppendorf tubes and centrifuged again (5 min, 2500 rpm in Hettich Mirco 200R centrifuge).
5. The combined pellet was resuspended in 100 μl LB medium and incubated without shaking 1 h at 30° C.
6. Then 1.9 ml of Synechocystis ΔleuB(sacB/Cm) strain grown in BG11 supplemented with 200 μg/ml leucine and 30 μg/ml Chloramphenicol at OD₇₅₀≈0.8 (mid-log phase) was added. The culture was slightly shaken and centrifuged (5 min, 2500 rpm in Hettich Mirco 200R centrifuge).
7. The pellet was resuspended in 50 μl BG-11 and dropped on a HATF (nitrocellulose membrane) filter (e.g. Millipore Durapore, 0.22 μm) which is located on top of a prepared plate (40 ml Bg11-agar+2 ml LB medium without antibiotics).
8. The plate was incubated for 2 days under low light conditions (25-35 μmol/m²*sec) at 28° C.
9. After incubation the bacteria were splashed of the filter with 200 μl BG-11, 1:10 diluted and carefully plated on a 1%-agar plate with 5 μg/ml Chloramphenicol. Selection took place via Chloramphenicol and the absence of leucine. Chloramphenicol was used in concentrations equal or higher to the maximal tolerated concentration of the incomplete segregated cyanobacterial strains.
10. After 7 days first transconjugants were visible. The control conjugation assay using only E. coli J53[RP4] and no E. coli pVZ-leuB₇₁₂₀-PpetJ-PDC/SynADH plasmid for complementation showed only very few colonies on the BG11-agar plate with Chloramphenicol (about 1/10 compared to the actual conjugation plate). Transconjugants were verified by PCR. A segregation test by PCR revealed still an incomplete segregation of the leuB gene inactivation.
11. Subsequently positive clones comprising the ethanologenic pVZ-leuB₇₁₂₀ plasmid were transferred stepwise to increasing Chloramphenicol concentrations (from 10 μg/ml, via 20 μg/ml to 30 μg/ml) in order to reach a complete segregation of the genomic leuB gene inactivation.
12. After two restreaks on BG11 plates with a final concentration of 30 μg/ml Chloramphenicol a complete segregation of several ΔleuB(SacB/Cm) clones was achieved (verified by PCR). Thus the final step, the removal of the sacB/Cm cassette could be started.
   B.3) Sucrose Mediated Removal of sacB/Cm Cassette from ΔleuB

In the third step via the same recombination platforms P1/P2 the sacB/Cm cassette was replaced. This step is driven by the loss of the sucrose sensitivity.

Procedure: Transformation of pJet-leuB P1/P2 (without sacB/Cm cassette) construct in the Synechocystis ΔleuB(sacB/Cm) pVZ-leuB₇₁₂₀-PpetJ-PDC/SynADH hybrid by natural competence
1. Cells of ΔleuB(sacB/Cm) pVZ-leuB₇₁₂₀-PpetJ-PDC/SynADH were cultivated to mid-log phase (OD₇₅₀≈1) in BG11 with 30 μg/ml Chloramphenicol on a rotary shaker at 28° C.
2. 12 ml of the culture were centrifuged at room temperature (10 min, 4.000 rpm in Hettich Rotina 240R Falcon tube centrifuge).
3. The supernatant was decanted and the pellet resuspended in the remaining medium (200 μl).
4. After transferring the 200 μl into a 1.5 ml Eppendorf tube, 0.5 μg of DNA (pJet-leuB P1/P2) were added. It followed an incubation time for at least 1 hour at room temperature, inverting the tube in between.
5. The suspension was plated on a HATF (nitrocellulose membrane) filter (e.g. Millipore Durapore, 0.22 μm) which is located on top of a BG11-1% agar plates without antibiotics. The cells were incubated at 28° C. under low light (25-35 μmol/m²*sec).
6. After 2 days the filter was transferred on BG11 plate supplemented with 2% sucrose and additional two 2 days later on a BG11 plate with 5% sucrose.
7. Most of the biomass on the filter died but single transformed blue-green colonies appeared within 15 days. These were picked and transferred to new BG11 plates with 5% sucrose.
8. To select the ΔleuB pVZ-leuB₇₁₂₀-PpetJ-PDC/SynADH clones from potential false positive clones with only sucrose resistance a test was performed comparing the same colonies on BG11+5% sucrose and on BG11+10 μg/ml Chloramphenicol plates. Intended clones should die in the presence of 10 μg/ml Chloramphenicol. To exclude wild type revertants a PCR control was done.
   C. Construction of an Antibiotic-Resistance-Cassette Free Manipulation System Based on the Zinc Tolerance Via smtAB for Generation of Ethanologenic Mutants in Synechococcus PCC7002
   C.1) SacB Mediated Replacement of smtAB

The sacB gene encodes the enzyme levansucrase from Bacillus subtilis that confers sucrose sensitivity on gram-negative bacteria. The sacB gene is site-directed (via platforms for homologous recombination) introduced into the cyanobacterial genome along with a gentamycin resistance cassette to replace the smtAB.

Procedure: Transformation of pJet-smtAB(SacB/Gm) construct in Synechococcus PCC 7002 by natural competence
1. Cells of Synechococcus were cultivated to mid-log phase (OD₇₅₀≈1) on a rotary shaker at 28° C.
2. 3 ml of the culture were centrifuged at room temperature (10 min, 4.000 rpm in Hettich Rotina 240R Falcon tube centrifuge).
3. The supernatant was decanted and the pellet resuspended in the remaining medium (200 μl).
4. After transferring the 200 μl into a 1.5 ml Eppendorf tube, min. 2 μg of linearized plasmid DNA pJet-smtAB(SacB/Gm) were added. It followed an incubation time for at least 1 hour at room temperature. Then up to 1 ml BG11 medium was added and the suspension was incubated overnight at 28° C. under 30 μmol/m2*sec light.
5. The suspension was then plated on a A+ plate (1% cyano agar) without antibiotics and incubated at 28° C. under low light (25-35 μmol/m²*sec).
6. After 2 days 300 μl gentamycin (5 mg/ml) were placed at one site of the agar plate to form a gradient of concentration within the plate as a result of diffusion.
7. Most of the biomass dies and turns yellow. After 5 weeks single transformed blue-green colonies appeared. These colonies were picked and transferred to new gentamycin plates for full segregation. The whole procedure takes about 2 month. Segregation was checked by PCR.

In a second step via the same recombination platforms the Gm/SacB cassette was replaced. This step is driven by the loss of the sucrose sensitivity.

Procedure: Transformation of pJet-delta smtAB construct in the Synechococcus PCC7002 ΔsmtAB(SacB/Gm) mutant by natural competence following the protocol as above up to step 5.
5. The suspension was plated on a HATF (nitrocellulose membrane) filter (e.g. Millipore Durapore, 0.22 μm) which is located on top of a BG11-1% cyanoagar plates supplemented with vitamin B12 (4 μg/L) without antibiotics. The cells were incubated at 28° C. under low light (25-35 μmol/m²*sec).
6. After 2 days little biomass from the filter was scraped off, diluted with BG11 and transferred on BG11 plates+vitamin B12 supplemented with 12% sucrose and 15% sucrose.
7. Most of the biomass on the filter died but single transformed blue-green colonies appeared within 15 days. These were picked and transferred to new BG11 plates+vitamin B12 and with 12% or 15% sucrose.
8. To select the ΔsmtAB clones with deleted Gm/SacB cassette from false positive clones with only sucrose resistance a test was performed comparing the same colonies on BG11 plates+vitamin B12 supplemented with 12% sucrose and on BG11 plates+vitamin B12 supplemented with 5 μg/ml gentamycin. In addition to exclude Synechococcus wild-type revertants with restored smtAB locus a PCR control was done.
   C.2) Complementation of Synechococcus ΔsmtAB Mutant with ziaA Gene and Ethanol Genes Encoded on Plasmid #1326 Via Conjugation

Procedure:
1. 3 ml overnight culture with E. coli J53[RP4] (50 μg/ml ampicillin and 20 μg/ml kanamycin) and 3 ml overnight culture with E. coli pVZhisB-PhspA-ziaA-P_{ind}-PDCdsrA-Prbc*-synADHdeg (plasmid #1326) were inoculated.
2. 250 μl of the overnight E. coli cultures were transferred in 10 ml fresh LB medium each, the E. coli J53[RP4] with appropriate antibiotics, and cultured in 100 ml Erlenmeyer flasks for 2.5 h shaking at 37° C.
3. Then the cells were sedimented by 8 min centrifugation in 15 ml falcon tubes at 2500 rpm and room temperature in a Hettich Rotina 240R centrifuge.
4. Both pellets were resuspended in 1 ml LB medium, with each other in a 2 ml Eppendorf tubes and centrifuged again (5 min, 2500 rpm in Hettich Mirco 200R centrifuge).
5. The combined pellet was resuspended in 100 μl LB medium and incubated without shaking 1 h at 30° C.
6. Then 1.9 ml Synechococcus ΔsmtAB with OD₇₅₀≈0.8 (mid-log phase) was added. The culture was slightly shaken and centrifuged (5 min, 2500 rpm in Hettich Mirco 200R centrifuge).
7. The pellet was resuspended in 50 μl BG-11 and dropped on a HATF (nitrocellulose membrane) filter (e.g. Millipore Durapore, 0.22 μm) which is located on top of a prepared plate (40 ml Bg11-cyano agar without antibiotics).
8. The plate was incubated for 2 days under low light conditions (25-35 μmol/m²*sec) at 28° C.
9. After incubation the bacteria were splashed of the filter with 200 μl BG11, 1:10 diluted and carefully plated on BG11 plates+vitamin B12 with 2 μM and 5 μM ZnSO₄. Selection took place via restored Zn²⁺ tolerance.
10. After 7 days first transconjugants were visible at all Zn²⁺ concentrations. The control conjugation assay using only E. coli J53[RP4] and no E. coli pVZhisB-PhspA-ziaA-P_{ind}-PDCdsrA-Prbc*-synADHdeg didn't show any colonies on the BG11-cyano agar plates with different ZnSO₄ concentration. Transconjugants were verified by PCR.

The scope of the protection of the invention is not limited to the example given herein above. The invention is embodied in each novel characteristic and each combination of characteristics, which particularly includes every combination of any features which are stated in the claims, even if this feature or this combination of features is not explicitly stated in the claims or in the examples.

What is claimed is:

1. A genetically enhanced cyanobacterium producing ethanol, comprising:
a) a genetically enhanced genome with a first gene inactivation in a first essential or conditionally essential gene of the cyanobacterium selected from the group consisting of smtAB, leuB, ziaRA, corRT, narB and pyrF; and
b) a first extrachromosomal plasmid harboring the first essential or conditionally essential gene and at least one first production gene for the production of ethanol, wherein the at least one first production gene for the production of ethanol encodes an enzyme selected from the group consisting of Adh, Pdc, AdhE and combinations thereof;
wherein the cyanobacterium lacks a functional gene conferring biocide resistance, further wherein the genome harbors more than one copy of the first essential or conditionally essential gene and wherein all copies of the first essential gene carry at least one gene inactivation, and further wherein said genetically enhanced cyanobacterium produces ethanol.

2. The genetically enhanced cyanobacterium of claim 1, wherein the first gene inactivation comprises an at least partial deletion of the first essential or conditionally essential gene.

3. The genetically enhanced cyanobacterium of claim 1, wherein the first gene inactivation comprises a complete deletion of the first essential or conditionally essential gene.

4. The genetically enhanced cyanobacterium of claim 1, wherein the first extrachromosomal plasmid comprises an endogenous plasmid of the cyanobacterium into which the first essential or conditionally essential gene and the at least one first production gene are integrated.

5. The genetically enhanced cyanobacterium of claim 1, wherein the at least one production gene comprises at least two first and second production genes coding for separate first and second production enzymes, which produce ethanol.

6. The genetically enhanced cyanobacterium of claim 5, wherein the first and second production genes are transcriptionally controlled by different promoters.

7. The genetically enhanced cyanobacterium of claim 1, wherein the essential or conditionally essential gene is transcriptionally controlled by a different promoter than the at least one first production gene.

8. The genetically enhanced cyanobacterium of claim 1, wherein the first essential gene encodes one of the following:
a) a first essential biocatalyst which is involved in the production of a first essential factor, which cannot promote complete genetic segregation of the cyanobacterium with regard to the first gene inactivation if present in the growth medium of the cyanobacteria; or
b) the first essential factor itself, which cannot promote complete genetic segregation of the cyanobacterium with regard to the first gene inactivation if present in the growth medium of the cyanobacteria.

9. The genetically enhanced cyanobacterium of claim 1, wherein the at least one first production gene is under the transcriptional control of an inducible or constitutive promoter.

10. The genetically enhanced cyanobacterium of claim 1, further comprising:
a) a second gene inactivation in a second essential or conditionally essential gene in the genome of the cyanobacterium, wherein the second essential or conditionally essential gene is different from the first essential or conditionally essential gene; and
b) at least one second production gene different from the first production gene;
wherein the second essential or conditionally essential gene and the second production gene are included on either the first extrachromosomal plasmid or on a second extrachromosomal plasmid.

11. The genetically enhanced cyanobacterium of claim 10, wherein the second production gene encodes an endogenous enzyme of the cyanobacterium, the expression of the endogenous enzyme resulting in an increased rate of production of ethanol compared to the respective cyanobacterium harboring the first production gene, but lacking the second production gene.

12. The genetically enhanced cyanobacterium of claim 11, wherein the endogenous enzyme is selected from the group consisting of enzymes of the glycolysis pathway, Calvin cycle, intermediate steps of metabolism, amino acid metabolism, the fermentation pathway and the citric acid cycle.

13. The genetically enhanced cyanobacterium of claim 1, wherein the first gene inactivation comprises a partial deletion of the first essential or conditionally essential gene and wherein the first extrachromosomal plasmid harbors a gene which is homologous to the first essential or conditionally essential gene inactivated by the first gene inactivation.

14. The genetically enhanced cyanobacterium of claim 1, wherein the first gene inactivation comprises a complete deletion of the first essential or conditionally essential gene and wherein the first extrachromosomal plasmid harbors the same first essential or conditionally essential gene inactivated by the first gene inactivation.

15. The genetically enhanced cyanobacterium of claim 1, wherein the first essential or conditionally essential gene included on the first extrachromosomal plasmid encodes an essential or conditionally essential biocatalyst, which is homologous or analogous to the biocatalyst encoded by the first essential or conditional essential gene in the genome of the cyanobacterium harboring the first gene inactivation.

16. A genetically enhanced cyanobacterium producing ethanol, comprising:
a) a genetically enhanced genome with a first gene inactivation in a first conditionally essential gene of the cyanobacterium; wherein the first conditionally essential gene is selected from the group consisting of narB, ziaA, smtA, corT, and combinations thereof; and
b) a first extrachromosomal plasmid harboring the first essential or conditionally essential gene and at least one first production gene for the production of ethanol;
wherein the cyanobacterium lacks a functional gene conferring biocide resistance.

17. A genetically enhanced cyanobacterium producing ethanol, comprising:
a) a genetically enhanced genome with a first gene inactivation in a first essential or conditionally essential gene of the cyanobacterium; and
b) a first extrachromosomal plasmid harboring the first essential or conditionally essential gene and at least one first production gene coding for Pdc enzyme and a second production gene coding for Adh enzyme, wherein a transcription terminator sequence is present between the first and second production gene and wherein the first production gene is controlled by an inducible promoter and the second production gene is under the transcriptional control of a constitutive promoter;
wherein the cyanobacterium lacks a functional gene conferring biocide resistance.

18. The genetically enhanced cyanobacterium of claim 17, showing an ethanol production rate from 0.024%(v/v)d⁻¹ to 0.032% (v/v)d⁻¹.

19. A method for producing ethanol, comprising the method steps of:
a) obtaining a genetically enhanced cyanobacterium producing ethanol, comprising
i) a genetically enhanced genome with a first gene inactivation in a first essential or conditionally essential gene of the cyanobacterium selected from the group consisting of smtAB, leuB, ziaRA, corRT, narB and pyrF; and
ii) a first extrachromosomal plasmid harboring the first essential or conditionally essential gene and at least one first production gene for the production of ethanol, wherein the at least one first production gene for the production of ethanol encodes an enzyme selected from the group consisting of Adh, Pdc, AdhE and combinations thereof; wherein the cyanobacterium lacks a functional gene conferring biocide resistance, further wherein the genome harbors more than one copy of the first essential or conditionally essential gene and wherein all copies of the first essential gene carry at least one gene inactivation;
b) culturing said genetically enhanced cyanobacterium in the absence of a biocide, the cyanobacterium producing ethanol; and
c) recovering the ethanol.

20. The method of claim 19, wherein the cyanobacterium harbors a first gene inactivation in a first conditionally essential gene and in method step a) the cyanobacteria are cultured under a condition rendering the first conditionally essential gene an essential gene.

21. A method for producing genetically enhanced cyanobacteria according to claim 1, comprising the method steps of:
i) transforming the cyanobacterium by introducing a first gene inactivation into a first essential or conditionally essential gene of the cyanobacterium; and
ii) introducing the first extrachromosomal plasmid harboring the first essential or conditionally essential gene and the at least one first production gene into the genetically enhanced cyanobacterium.

22. The method of claim 21, wherein method step i) comprises replacing at least a part of the first essential or conditionally essential gene of the cyanobacterium with a recombinant nucleic acid sequence, thereby creating the first gene inactivation.

23. The method of claim 21, wherein method step i) comprises the following substeps:
i1) transforming the cyanobacteria with a first recombinant nucleic acid sequence, wherein the nucleic acid sequence comprises a first selectable gene conferring resistance to a selectable marker and a second counterselectable gene conferring sensitivity to a counterselectable marker;
i2) selecting for transformed cyanobacteria by subjecting the cyanobacteria to the selectable marker;
i3) transforming the cyanobacteria obtained from step i2) with a second recombinant nucleic acid sequence lacking the first selectable and second counterselectable gene by replacing at least a part of the first recombinant nucleic acid sequence, thereby creating transformed cyanobacteria lacking a functional first selectable and functional second counterselectable gene;
i4) selecting for transformed cyanobacteria from step i3) via subjecting the cyanobacteria to the counterselectable marker; and
i5) introducing the extrachromosomal plasmid into the cyanobacteria obtained from step i4).

24. The method of claim 21, wherein the genome of the cyanobacteria harbors more than one copy of the first essential gene, the essential gene encoding a biocatalyst for the production of a first essential factor, which cannot promote complete genetic segregation of the cyanobacterium with regard to the first gene inactivation if present in the growth medium and which includes the following substeps:
i′1) first gene inactivations are created in not all copies of the first essential gene by replacing at least parts of the first essential gene by a first recombinant nucleic acid;
i′2) selecting for the cyanobacteria obtained in substep i′1) by subjecting the cyanobacteria to the first selectable marker,
followed by step ii) of introducing the first extrachromosomal plasmid harboring the first essential or conditionally essential gene and the at least one first production gene into the genetically enhanced cyanobacteria of substep I′2);
i′3) subjecting the cyanobacteria from step ii) to concentrations of the first selectable marker higher than the concentration used in substep i′2);
i′4) transforming the cyanobacteria obtained from step i′3) with the second recombinant nucleic acid; and
i′5) selecting for cyanobacteria obtained in substep i′4) by subjecting the cyanobacteria to the counterselectable marker.


SEQUENCE LISTING (23 sequences)

SEQ ID NO: 1 - DNA, 12701 bp, Artificial
  plasmid # 309 pVZ325-PpetJ-PDC/SynADH
tcgacgggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcaactttgacagattgtcttttgactttgcctggaccgcctcccataataccttcgcgtcttgaagactttatccttgaaaggagaactaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctctctggataaaactaataaactctattacccatgattaaagcctacgctgccctggaagccaacggaaaactccaaccctttgaatacgaccccggtgccctgggtgctaatgaggtggagattgaggtgcagtattgtggggtgtgccacagtgatttgtccatgattaataacgaatggggcatttccaattaccccctagtgccgggtcatgaggtggtgggtactgtggccgccatgggcgaaggggtgaaccatgttgaggtgggggatttagtggggctgggttggcattcgggctactgcatgacctgccatagttgtttatctggctaccacaacctttgtgccacggcggaatcgaccattgtgggccactacggtggctttggcgatcgggttcgggccaagggagtcagcgtggtgaaattacctaaaggcattgacctagccagtgccgggccccttttctgtggaggaattaccgttttcagtcctatggtggaactgagtttaaagcccactgcaaaagtggcagtgatcggcattgggggcttgggccatttagcggtgcaatttctccgggcctggggctgtgaagtgactgcctttacctccagtgccaggaagcaaacggaagtgttggaattgggcgctcaccacatactagattccaccaatccagaggcgatcgccagtgcggaaggcaaatttgactatattatctccactgtgaacctgaagcttgactggaacttatacatcagcaccctggcgccccagggacatttccactttgttggggtggtgttggagcctttggatctaaatctttttccccttttgatgggacaacgctccgtttctgcctccccagtgggtagtcccgccaccattgccaccatgttggactttgctgtgcgccatgacattaaacccgtggtggaacaatttagctttgatcagatcaacgaggcgatcgcccatctagaaagcggcaaagcccattatcgggtagtgctcagccatagtaaaaattagctctgcaaaggttgcttctgggtccgtggaatggtcaaacggagtcgatctcagttttgatacgctctatctggaaagcttgacattcgatctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagaccgagctcgaattggccgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaattcgagcatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagctctagag

SEQ ID NO: 2 - DNA, 12696 bp, Artificial
  plasmid # 550 pVZ325-PpetJ-PDC-Prbc*SynADH(deg)
tcgacgggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcaactttgacagattgtcttttgactttgcctggaccgcctcccataataccttcgcgtcttgaagactttatccttgaaaggagaactaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctcggtacccaaactagtcgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagaccgagctcgaattggccgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaattcgagcatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagctctagag

SEQ ID NO: 3 - DNA, 10683 bp, Artificial
  plasmid # 570 pVZleuB-PpetJ-PDC/SynADH
tcgacgggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcaactttgacagattgtcttttgactttgcctggaccgcctcccataataccttcgcgtcttgaagactttatccttgaaaggagaactaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctctctggataaaactaataaactctattacccatgattaaagcctacgctgccctggaagccaacggaaaactccaaccctttgaatacgaccccggtgccctgggtgctaatgaggtggagattgaggtgcagtattgtggggtgtgccacagtgatttgtccatgattaataacgaatggggcatttccaattaccccctagtgccgggtcatgaggtggtgggtactgtggccgccatgggcgaaggggtgaaccatgttgaggtgggggatttagtggggctgggttggcattcgggctactgcatgacctgccatagttgtttatctggctaccacaacctttgtgccacggcggaatcgaccattgtgggccactacggtggctttggcgatcgggttcgggccaagggagtcagcgtggtgaaattacctaaaggcattgacctagccagtgccgggccccttttctgtggaggaattaccgttttcagtcctatggtggaactgagtttaaagcccactgcaaaagtggcagtgatcggcattgggggcttgggccatttagcggtgcaatttctccgggcctggggctgtgaagtgactgcctttacctccagtgccaggaagcaaacggaagtgttggaattgggcgctcaccacatactagattccaccaatccagaggcgatcgccagtgcggaaggcaaatttgactatattatctccactgtgaacctgaagcttgactggaacttatacatcagcaccctggcgccccagggacatttccactttgttggggtggtgttggagcctttggatctaaatctttttccccttttgatgggacaacgctccgtttctgcctccccagtgggtagtcccgccaccattgccaccatgttggactttgctgtgcgccatgacattaaacccgtggtggaacaatttagctttgatcagatcaacgaggcgatcgcccatctagaaagcggcaaagcccattatcgggtagtgctcagccatagtaaaaattagctctgcaaaggttgcttctgggtccgtggaatggtcaaacggagtcgatctcagttttgatacgctctatctggaaagcttgacattcgatctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagtttttcagcaagatggatcctataatctgcaagcatttaccaaaggagaattcagctatgactcagaactatcgaattaccctactccctggcgatggtattggccctgaaattatggcagtggcggtagatgtgctgaaagtcgtagggcagcaatttgatattcagtttgatttccaagaagctttgattggtggtgcagcaattgacgcgacaggcgaacctctgccatcggctactttagatacttgtcgccacagtgatgctgtattactcgctgctattggtggttataagtgggattccttaccccctcaccaacgcccggaagggggtttgttgggattgcgtgcgggattagaattatttgccaatttacgcccagcccaaattttaccgcagttaatcgacgcttcgactttgaagcgagaagttgtggaaggtgtagatattatggtggtgcgtgaactcactggtgggatttactttggtaaacccagaggaatctttactactgagactggcgagaaacgtggcgttaatacgatggtttatacagaatcggaaatcgacagaattggccggatagcttttgagactgcgcggaaacgtcggggtaaactctgttcagtagataaagccaatgtattagatgtatcccaattgtggcgcgatcgcatcaccaatctttcccaagaatacccagacgtggaactttcccatctatatgtagataatgccgccatgcaattggtacgcgctcccaagcagttcgataccatcgttacaggcaacttgtttggggatattctctcggatgcggctgccatgcttacaggtagtattggaatgttaccctcggctagtcttggggcttctgggccgggagtctttgaaccagtccacggttctgccccagatatcgcaggacaggataaagccaaccccctcgcccaagttttgagtgcggcgatgatgttgcgttacgctttagaccaaccacaagcagcagacaaaatagaacaggctgtattacaagttttagagcaaggtgatcgcacaggggatataatttcagtgggtatgaatcttttgggttgccggggtatgggtgactccctcataaaggcattagcaaaatcataatttcccggaacgctcggttgccgccgggcgttttttattccatcgatatctgcagatctttctagag

SEQ ID NO: 4 - DNA, 8484 bp, Artificial
  plasmid # 675 pJet-leuB-P1/P2-Cm-SacB
ctagacgttttcttcactttatgtaccctttccctaaattattgggggaagggtatttgctgggaggtgggaggcgatcgccagtggctgaagtttctaatctgcgtccccaatccactacactggggaagcttgtggaatccattcccaaagcccgtttaaatttacttatttgatcaaagttttttcagttcttccactaccatgtcccagacctataacgtaaccctgctccccggtgatggcattggccccgaaattatggcggtggcggtggcggtgttaggcaaagtggccgaccagtttggctttgcgtttaattttcaagaggctttaattggcggagcggccattgatgccacagggcaacccctaccggaggcaaccctagcccaagcaaaggatagtgatgcggtactcttagcggcgatcgggggttatgcctgggacaatttaccccgttctcaaaggccggaaactggtttgcttgccattcgggaagggttggggctatttgccaatttacgtcctgcgacgatcttcccccaattgattgacgcttccagcctgaaacgggaagtggtggaaggggtggatattatggtggtgcgggaactaaccggcggcatttattttggtaaaccgaagggtattttcgagacggaaacggcccggattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcgtagtcgacggtatcgataagcttgatatcgaattcctgcagccaagctagacctaggccttaagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggaggtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatatgaactatataaaagcaggcaaatggctaaccgtattcctaaccttttggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagcctgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcaggccggcattaatatcttgctgaaaaactcgagcgctacggtttggatcaaccccaggcggccgatcgccttgaggatgcagtgaaaaaagttttagaacagggttaccgcaccggtgatattctttctcctggcacacaattggtgggctgtcggcaaatgggagagcaattgttgagcatcctagatgaaatgtaacgaccattgcccttggctaaaaactagacaaactctgccctattaatttccccacggccaggcggttcgggtcaaattagatcttggtcggataactaagaatcttctggggtggcccttacgtcaaaccctattgttatttatgatgatggctaacctggctgacatttttcggtcaatggctacaacacatatgcgtcctgagtggggagtttgttatggtttttattcctgatgagagcgattttgatgtggagtacaggagcgtacccatgcccctagaccccaaaatgttgcggatggcccgacgcatttatcgcagttaccgcatgctccacccccgatcccaaaagcagcctctgggcattgctatccataaagaaacccaccggggtcaactgatgttcaatcgtcagccagtgctgttgccaggggaatgtttcgtacccttaaaccaactggaagcggaagcccaggtagtttgaactagcttcatccctctgttatgaccgaatcttcgcccctagcaccatcgacggccccggcaacccgcaaactttggttagcggcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttccatggcagctgagaatattgtaggagatcttctagtgccatcaattccaagaaagctctttaaaaactctctttgctccccagagttgggcaaaatcggagttaat

SEQ ID NO: 5 - DNA, 10502 bp, Artificial
  plasmid # 802 pVZpyrF-PpetJ-PDC-Prbc*-SynADH(deg)
tcgacgggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcaactttgacagattgtcttttgactttgcctggaccgcctcccataataccttcgcgtcttgaagactttatccttgaaaggagaactaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctcggtacccaaactagtcgatcgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggaataaaaaacgcccggcggcaaccgagcgttcataccactgttgttaactcttgagaaatgcgtttccaggctaactcaggttcagcagcagcagtaattggacgaccaatcactaaataatcagcccctgcttggatggcttgggagggggtgagcgatcgcttttgatctcccgcttctgcccatgttgggcgtacccccggacaaaccaacacgaaatcatccccgcaaatttggcgtaaatgtgccacttcttggggtgaacacactgccccatctaaccccatctcctgcgccattagagccatatctaaagcatattctggcaattctgaaggaattttcaaatctaacgccaactgtctggcagaaatactggttaacaccgtcacagcgattaacttgggcggtttcacaccagcttgtgcagccccttcctctaccgcctcttttgcagccttcagggcatctctaccagaggtagcatggattgtcatcaaatccaccccgtaaccagccgccgcacgacaagccccagccaccgtattggggatgtcgtgaaacttcaaatcgagaaagatgcgcttttgtctagatttcagtacttctagaattctggggccggaactagtaaacaattccaacccaaccttccaaaaagtcaccgactctagcttatcaatgagagcgatcgcactctccgtatctggcacatccaaagccacaattattttgtcattactcattaatttcctcctacacccttacaccattataacaggatcctgtgtggaaggaattgattacccttgaatggttctatcttaaaatttccccttccaggttagattcggttttcaggaaagaaggtggggggattgccgaaattacatttctagccgcaatttttagtaaaaaaaagatgagtttttacctcaccttaagtaaatatttgagtggcaaaacaaaatggtaaaaatagctaagcttccaccgccctatggatttttggaaggaagtcttaggttgtg

SEQ ID NO: 6 - DNA, 9082 bp, Artificial
  plasmid # 814 pJet-ziaRA-P1/P2-SacB
ctagatttgagtgcggttatgccaaggtaattgtgatcgccggactcttgctcggtacgttgccgccttttttgctgggctggagttgggaagagacgatctaccgtgccttaatttttctcgtggtggcttctccctgtgcgctaatggcatcaattatgccggccttactttctggtattgccaatggagcgcgtcaaggcattttattcaaaaacggggcgcagttggagcggattggacgagtacgggtgattgcttttgataaaactggaacattgaccactggaaaacctgaagttgtgaacattctcgccactcagccatccaccgacaaacttctgcaaatcgcggcggccctggaaagtttatcggaacatcccatcggagaggcgatcgccgacttcacccgtcaacagaaccaagcctgggcaacagccaggaacgttcaagcgcaggcgggacagggcattatcggagacattgagggccaacaggcgatagttggtaaagccgtgtttgttcaggcccaggtcaatcatgttgctacaaacctgatagagcaaagtcagcagtgggaagctgaaggtaaaaccgtcgtctgggttgcctacgctggagagattttaggtctcattgccgtggcagatactgtacgacccaccgccgcccaggcgatcgcccgattaaaacgattaggaattgagcgaattgtcatgctaactggggataactcacgtaccgcccacagcattgctcaacaggtgggagtcaatcaggtttatgcagaacttttgcccgaagataaagtggatgtgattcgccaactccagaagcagtatcaatccgtcgccatggtgggagacggcatcaatgatgcacccgccttagctcaggcatctgttggtattgcgatgggggccgctggtagtgatgtggccttagaaaccgcagatattgtcttaatggcggatcgtttggagcggttagaacacgccattcgtttgggtcgtcgcgctcaaggagtggtcaaacaaaacattgtgtttgcccttggttttgtgatgattctgttgattgcaaactttgcgggtaatatcacgctgccctttggggttttggggcatgaaggctcgacggtaatcgtaacgttgagtggattgcggttactaagagggtaacccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatgctcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccaattcgagctcggtctcgacggtatcgataagcttgatatcgaattcctgcagccaagctagacctaggccttaagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggaggtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatatgaactatataaaagcaggcaaatggctaaccgtattcctaaccttttggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagcctgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcaggccggcattaatatcttgctgaaaaactcgagccctgttaggtgagatttttggtacactctcgggactgttggtatggtggcggacatattgataagcgaatggtgaataaacttccctgacccaattcactctctacagtcaaaacaccttgatgcttaacggcgatcgcctgggcaatggcaaggcctaatcctgcccctccccgttgtcgagaccgggccgtgtccacccgataaaaacggtcaaaaacccggctttgattctccgaagcaatgccaatgcccgtatcttggactttgatgatggcttgctgtttatcggtctcaagcatgacggttacctcgccccccgttggggtgtagtgaatggcattactgattaaattacccaccaaacgataaagctgttcttcttctcccctgacccagatatttgcttgattgtctaacttcgcagataacaataccccagcggcgatcgccaaactggcaaattcttcggttaaatcttcaactaaatcattgagacaacaaagggtaaactgggtcggattaactgttgttaaatccatgcgggagaggagtaataaatcatggattaaatggctgagacgataattttgtcgttttaaggtttgcaaggtgctatgggtttcttcagcattaggttctgcatttaatgtcgtttccaacgttgcctgaatcgccgtaatcggtgtacgtaattcgtgggcaatatcagcggtgaactgctggatttgttggtaggaacgataaaccggctccatggctaagcctgctaaccaccaactagcaccaccgaccacaatcatcacaatgggtaaaccccaaactaaaaagccttggatcgtgtgcaaatgatggtcatattccaccaaggagcgccccacctttaaatagccccaaggttcaccagtggtggtttttaacagcaaagaaacttggtgatagcggttccctttttgatccgttaatggtttttgacgggtggtttcccgctcaaaggctaagcctgggggattttcccctgccgttgctaataattgaccgtttaagtccaaaaaccggacataatagcccggctgttgggttgcgttgagaatatgcctccgttgaggcgacctagggcaagctactgtacctaagcaaagattaggcaaaatttgtttaactgagggctctaactgccctggttgttgtaatagtggttccaatccatcatggagagtccccgctagggaagtaagctcttgatccagcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttccatggcagctgagaatattgtaggagatctt

SEQ ID NO: 7 - DNA, 13363 bp, Artificial
  plasmid # 819 pVZhisB-narB-PpetJ-PDC-Prbc*SynADH(deg)
tcgacgggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcaactttgacagattgtcttttgactttgcctggaccgcctcccataataccttcgcgtcttgaagactttatccttgaaaggagaactaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctcggtacccaaactagtcgatcgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgctagaacggatatgctaaatgcgtaaactgcatatcccttggctgagtgtaatttacgttacaaattttaacgaaacgggaaccctatattgatctctacgtttatggaggactgaccatatgaattcactgccaatattttaatggattcaccagctattcttcccaccaccagaaccctttgtccttactgtggagttggttgtggtttggaggctgttgctagtccccaaaagtcggtagttgatgccggtcatgcccataaaattagaggcgatcgccagcatccttccagccaaggcatggtctgcgtcaaaggagctaccgtaatggaatccatggataaacagcgattactttaccccatgtttcggtcaagtttagatcagccgtggcaacaaattagttgggaagcagccctagaaattgttgttgataaaattcagcaagttaaacaaactttgggggtaagtggtttatgtatgtacgcctctgggcaaatgcagacagaggattactacgtggcccaaaaattatttaaaggttgtttgggtaccaataattttgataccaattctcgtctttgcatgtcttcggctgtgtctgcctatagcctaagctttggttccgatggtccgccttgctgttatgaagacttagaaataaccgattgcgcttttttaattggtactaatacagcggactgccatcccattatttttaatcgtttacgcaaacaccacaaacaaaatccccacgttaaattaattgtggtggacccccgttgtactgccaccgcagaagttgcggacttacatttagcgatcaatcctggcagtgacattactttactccatggcattgcctatttgctaaaaaagtggaatttaattgatcaaaaatttattgataatcacacccaagattttgagcagtattgtcaagttattgatcattatcccccggaaaaagttacccaaatttgtggcattagtttagaacaattagaaaccgccgctcactattggggcaatgccaaaacagtgctttccctttggtccatgggaatgaatcaatcttttcaaggcacagctaaaggtcgttgtttaattaatctccatttactcactggtcagattgggaaaccaggcagtggacctttttccctcactggtcagcccaacgccatggggggtcgggaagcagggggattgtcccatctgttgcctggctatcgttcaattaaaaatcctcaacaccgccaagaggtagaacaactttggcagatttcccctggccgtattaatccagaaccaggtttatcagcctgggaaatgttcatgggattggaaaatcaacaggttggtttcctatggattgtggcgactaaccctgttgttagtatgcctgatttagaaagggttaaaaaggctctgcaacaatcaacttttacgattcatcaggatgcctattcccccactgaaacggcagaatacgcccatttgttattgccagcagctcaatggagcgaaaaaactggcacaatgaccaattcagaacggagagttaccctttctcctgcgtttcgctctcctcctggggaagcaagacccgactgggaaatttttgctgaggtaggacgtagattaggttttgaaaatcaatttaattttgttgattcagccgcagtacatcgggaatatgtgcaactaactgctgaaagattatgtgaccaatctggtgtcagttatgaaaagttgcaaaagctggggccgctgcaatggccctgtcgtcaatcagatcaagaaagtcaacttttatcaactaaaagactttacaccgactataaattttgcacagaaaatggtcgggcaaacttttgcttagaccacagtcggggattagctgagccggtcgatccaaactatccttttgtcctcaccaatggtcgtctttatggtcattggcatacccaaacccgcactggccacattgagaaaatcaaaaaaatgcatcccaaacctatattggaaatgcatcccaaggacgcagaaaaattgggtattaaaagtcaggatttagttgcgattaaatctcgacggggttctgctcagctagaagttctagttactagagccattgcccccggtacggtttttatgcccatgcactggggttttttgtgggacgataatgctgaagttaatagtttgacccatgccactgcttgccctatttctaaacaaccggaattaaaagcctgcgccgttaatattacaccagtttaatcaaacaataagcaattaattgtcaggataaaaatctaagcttaaaatttggtcaggaatatatgagtattgttgaggaaatcgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgacacaacctaagacttccttccaaaaatccatagggcggtggaagcttagctatttttaccattttgttttgccactcaaatatttacttaaggtgaggtaaaaactcatcttttttttactaaaaattgcggctagaaatgtaatttcggcaatccccccaccttctttcctgaaaaccgaatctaacctggaaggggaaattttaagatagaaccattcaagggtaatcaattccttccacacaggatccgcgctttaaagcctccagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttgatgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaaaacgctcggttgccgccgggcgttttttattctagag

SEQ ID NO: 8 - DNA, 13404 bp, Artificial
  plasmid # 820 pVZhisB-narB-PpetJ-PDC/SynADH standard
tcgacgggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcaactttgacagattgtcttttgactttgcctggaccgcctcccataataccttcgcgtcttgaagactttatccttgaaaggagaactaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctcggtacccaaactagttctggataaaactaataaactctattacccatgattaaagcctacgctgccctggaagccaacggaaaactccaaccctttgaatacgaccccggtgccctgggtgctaatgaggtggagattgaggtgcagtattgtggggtgtgccacagtgatttgtccatgattaataacgaatggggcatttccaattaccccctagtgccgggtcatgaggtggtgggtactgtggccgccatgggcgaaggggtgaaccatgttgaggtgggggatttagtggggctgggttggcattcgggctactgcatgacctgccatagttgtttatctggctaccacaacctttgtgccacggcggaatcgaccattgtgggccactacggtggctttggcgatcgggttcgggccaagggagtcagcgtggtgaaattacctaaaggcattgacctagccagtgccgggccccttttctgtggaggaattaccgttttcagtcctatggtggaactgagtttaaagcccactgcaaaagtggcagtgatcggcattgggggcttgggccatttagcggtgcaatttctccgggcctggggctgtgaagtgactgcctttacctccagtgccaggaagcaaacggaagtgttggaattgggcgctcaccacatactagattccaccaatccagaggcgatcgccagtgcggaaggcaaatttgactatattatctccactgtgaacctgaagcttgactggaacttatacatcagcaccctggcgccccagggacatttccactttgttggggtggtgttggagcctttggatctaaatctttttccccttttgatgggacaacgctccgtttctgcctccccagtgggtagtcccgccaccattgccaccatgttggactttgctgtgcgccatgacattaaacccgtggtggaacaatttagctttgatcagatcaacgaggcgatcgcccatctagaaagcggcaaagcccattatcgggtagtgctcagccatagtaaaaattagctctgcaaaggttgcttctgggtccgtggaatggtcaaacggagtcgatctcagttttgatacgctctatctggaaagcttgacattcgatcaattggatccgatacgtaacgcgtctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgctagaacggatatgctaaatgcgtaaactgcatatcccttggctgagtgtaatttacgttacaaattttaacgaaacgggaaccctatattgatctctacgtttatggaggactgaccatatgaattcactgccaatattttaatggattcaccagctattcttcccaccaccagaaccctttgtccttactgtggagttggttgtggtttggaggctgttgctagtccccaaaagtcggtagttgatgccggtcatgcccataaaattagaggcgatcgccagcatccttccagccaaggcatggtctgcgtcaaaggagctaccgtaatggaatccatggataaacagcgattactttaccccatgtttcggtcaagtttagatcagccgtggcaacaaattagttgggaagcagccctagaaattgttgttgataaaattcagcaagttaaacaaactttgggggtaagtggtttatgtatgtacgcctctgggcaaatgcagacagaggattactacgtggcccaaaaattatttaaaggttgtttgggtaccaataattttgataccaattctcgtctttgcatgtcttcggctgtgtctgcctatagcctaagctttggttccgatggtccgccttgctgttatgaagacttagaaataaccgattgcgcttttttaattggtactaatacagcggactgccatcccattatttttaatcgtttacgcaaacaccacaaacaaaatccccacgttaaattaattgtggtggacccccgttgtactgccaccgcagaagttgcggacttacatttagcgatcaatcctggcagtgacattactttactccatggcattgcctatttgctaaaaaagtggaatttaattgatcaaaaatttattgataatcacacccaagattttgagcagtattgtcaagttattgatcattatcccccggaaaaagttacccaaatttgtggcattagtttagaacaattagaaaccgccgctcactattggggcaatgccaaaacagtgctttccctttggtccatgggaatgaatcaatcttttcaaggcacagctaaaggtcgttgtttaattaatctccatttactcactggtcagattgggaaaccaggcagtggacctttttccctcactggtcagcccaacgccatggggggtcgggaagcagggggattgtcccatctgttgcctggctatcgttcaattaaaaatcctcaacaccgccaagaggtagaacaactttggcagatttcccctggccgtattaatccagaaccaggtttatcagcctgggaaatgttcatgggattggaaaatcaacaggttggtttcctatggattgtggcgactaaccctgttgttagtatgcctgatttagaaagggttaaaaaggctctgcaacaatcaacttttacgattcatcaggatgcctattcccccactgaaacggcagaatacgcccatttgttattgccagcagctcaatggagcgaaaaaactggcacaatgaccaattcagaacggagagttaccctttctcctgcgtttcgctctcctcctggggaagcaagacccgactgggaaatttttgctgaggtaggacgtagattaggttttgaaaatcaatttaattttgttgattcagccgcagtacatcgggaatatgtgcaactaactgctgaaagattatgtgaccaatctggtgtcagttatgaaaagttgcaaaagctggggccgctgcaatggccctgtcgtcaatcagatcaagaaagtcaacttttatcaactaaaagactttacaccgactataaattttgcacagaaaatggtcgggcaaacttttgcttagaccacagtcggggattagctgagccggtcgatccaaactatccttttgtcctcaccaatggtcgtctttatggtcattggcatacccaaacccgcactggccacattgagaaaatcaaaaaaatgcatcccaaacctatattggaaatgcatcccaaggacgcagaaaaattgggtattaaaagtcaggatttagttgcgattaaatctcgacggggttctgctcagctagaagttctagttactagagccattgcccccggtacggtttttatgcccatgcactggggttttttgtgggacgataatgctgaagttaatagtttgacccatgccactgcttgccctatttctaaacaaccggaattaaaagcctgcgccgttaatattacaccagtttaatcaaacaataagcaattaattgtcaggataaaaatctaagcttaaaatttggtcaggaatatatgagtattgttgaggaaatcgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgacacaacctaagacttccttccaaaaatccatagggcggtggaagcttagctatttttaccattttgttttgccactcaaatatttacttaaggtgaggtaaaaactcatcttttttttactaaaaattgcggctagaaatgtaatttcggcaatccccccaccttctttcctgaaaaccgaatctaacctggaaggggaaattttaagatagaaccattcaagggtaatcaattccttccacacaggatccgcgctttaaagcctccagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttgatgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaaaacgctcggttgccgccgggcgttttttattctagag

SEQ ID NO: 9 - DNA, 5367 bp, Artificial
  plasmid # 821 pJET-ziaRA-P1/P2 standard
ctagatttgagtgcggttatgccaaggtaattgtgatcgccggactcttgctcggtacgttgccgccttttttgctgggctggagttgggaagagacgatctaccgtgccttaatttttctcgtggtggcttctccctgtgcgctaatggcatcaattatgccggccttactttctggtattgccaatggagcgcgtcaaggcattttattcaaaaacggggcgcagttggagcggattggacgagtacgggtgattgcttttgataaaactggaacattgaccactggaaaacctgaagttgtgaacattctcgccactcagccatccaccgacaaacttctgcaaatcgcggcggccctggaaagtttatcggaacatcccatcggagaggcgatcgccgacttcacccgtcaacagaaccaagcctgggcaacagccaggaacgttcaagcgcaggcgggacagggcattatcggagacattgagggccaacaggcgatagttggtaaagccgtgtttgttcaggcccaggtcaatcatgttgctacaaacctgatagagcaaagtcagcagtgggaagctgaaggtaaaaccgtcgtctgggttgcctacgctggagagattttaggtctcattgccgtggcagatactgtacgacccaccgccgcccaggcgatcgcccgattaaaacgattaggaattgagcgaattgtcatgctaactggggataactcacgtaccgcccacagcattgctcaacaggtgggagtcaatcaggtttatgcagaacttttgcccgaagataaagtggatgtgattcgccaactccagaagcagtatcaatccgtcgccatggtgggagacggcatcaatgatgcacccgccttagctcaggcatctgttggtattgcgatgggggccgctggtagtgatgtggccttagaaaccgcagatattgtcttaatggcggatcgtttggagcggttagaacacgccattcgtttgggtcgtcgcgctcaaggagtggtcaaacaaaacattgtgtttgcccttggttttgtgatgattctgttgattgcaaactttgcgggtaatatcacgctgccctttggggttttggggcatgaaggctcgacggtaatcgtaacgttgagtggattgcggttactaagagggtaaccctcgagccctgttaggtgagatttttggtacactctcgggactgttggtatggtggcggacatattgataagcgaatggtgaataaacttccctgacccaattcactctctacagtcaaaacaccttgatgcttaacggcgatcgcctgggcaatggcaaggcctaatcctgcccctccccgttgtcgagaccgggccgtgtccacccgataaaaacggtcaaaaacccggctttgattctccgaagcaatgccaatgcccgtatcttggactttgatgatggcttgctgtttatcggtctcaagcatgacggttacctcgccccccgttggggtgtagtgaatggcattactgattaaattacccaccaaacgataaagctgttcttcttctcccctgacccagatatttgcttgattgtctaacttcgcagataacaataccccagcggcgatcgccaaactggcaaattcttcggttaaatcttcaactaaatcattgagacaacaaagggtaaactgggtcggattaactgttgttaaatccatgcgggagaggagtaataaatcatggattaaatggctgagacgataattttgtcgttttaaggtttgcaaggtgctatgggtttcttcagcattaggttctgcatttaatgtcgtttccaacgttgcctgaatcgccgtaatcggtgtacgtaattcgtgggcaatatcagcggtgaactgctggatttgttggtaggaacgataaaccggctccatggctaagcctgctaaccaccaactagcaccaccgaccacaatcatcacaatgggtaaaccccaaactaaaaagccttggatcgtgtgcaaatgatggtcatattccaccaaggagcgccccacctttaaatagccccaaggttcaccagtggtggtttttaacagcaaagaaacttggtgatagcggttccctttttgatccgttaatggtttttgacgggtggtttcccgctcaaaggctaagcctgggggattttcccctgccgttgctaataattgaccgtttaagtccaaaaaccggacataatagcccggctgttgggttgcgttgagaatatgcctccgttgaggcgacctagggcaagctactgtacctaagcaaagattaggcaaaatttgtttaactgagggctctaactgccctggttgttgtaatagtggttccaatccatcatggagagtccccgctagggaagtaagctcttgatccagcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttccatggcagctgagaatattgtaggagatctt

SEQ ID NO: 10 - DNA, 4489 bp, Artificial
  plasmid # 856 pJET-leuB-P1/P2 standard
ctagacgttttcttcactttatgtaccctttccctaaattattgggggaagggtatttgctgggaggtgggaggcgatcgccagtggctgaagtttctaatctgcgtccccaatccactacactggggaagcttgtggaatccattcccaaagcccgtttaaatttacttatttgatcaaagttttttcagttcttccactaccatgtcccagacctataacgtaaccctgctccccggtgatggcattggccccgaaattatggcggtggcggtggcggtgttaggcaaagtggccgaccagtttggctttgcgtttaattttcaagaggctttaattggcggagcggccattgatgccacagggcaacccctaccggaggcaaccctagcccaagcaaaggatagtgatgcggtactcttagcggcgatcgggggttatgcctgggacaatttaccccgttctcaaaggccggaaactggtttgcttgccattcgggaagggttggggctatttgccaatttacgtcctgcgacgatcttcccccaattgattgacgcttccagcctgaaacgggaagtggtggaaggggtggatattatggtggtgcgggaactaaccggcggcatttattttggtaaaccgaagggtattttcgagacggaaacggcccggattaaattccaacatggatgctgatttatatgggtataaatgggctcgggcattaatatcttgctgaaaaactcgagcgctacggtttggatcaaccccaggcggccgatcgccttgaggatgcagtgaaaaaagttttagaacagggttaccgcaccggtgatattctttctcctggcacacaattggtgggctgtcggcaaatgggagagcaattgttgagcatcctagatgaaatgtaacgaccattgcccttggctaaaaactagacaaactctgccctattaatttccccacggccaggcggttcgggtcaaattagatcttggtcggataactaagaatcttctggggtggcccttacgtcaaaccctattgttatttatgatgatggctaacctggctgacatttttcggtcaatggctacaacacatatgcgtcctgagtggggagtttgttatggtttttattcctgatgagagcgattttgatgtggagtacaggagcgtacccatgcccctagaccccaaaatgttgcggatggcccgacgcatttatcgcagttaccgcatgctccacccccgatcccaaaagcagcctctgggcattgctatccataaagaaacccaccggggtcaactgatgttcaatcgtcagccagtgctgttgccaggggaatgtttcgtacccttaaaccaactggaagcggaagcccaggtagtttgaactagcttcatccctctgttatgaccgaatcttcgcccctagcaccatcgacggccccggcaacccgcaaactttggttagcggcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttccatggcagctgagaatattgtaggagatcttctagtgccatcaattccaagaaagctctttaaaaactctctttgctccccagagttgggcaaaatcggagttaat

SEQ ID NO: 11 - DNA, 14006 bp, Artificial
  plasmid # 864 pVZhisB-ziaRA-PpetJ-PDC-Prbc*-SynADH(deg)
tcgacgggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcaactttgacagattgtcttttgactttgcctggaccgcctcccataataccttcgcgtcttgaagactttatccttgaaaggagaactaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctcggtacccaaactagtcgatcgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggatccgcgctttaaagcctccagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttgatgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaaaacgctcggttgccgccgggcgttttttattctagagtggattgcggttactaagagggtaattaattaatgatggaattaagaacctgtttataaagtctgattctgccccctaaatcccccaatactgggggactttgacttagtttccccccaaacttggtgggccaggggggcttttcaaacacgctctaagccctgttagcatacatcatcactaatacaccaaccaaggcgatcgccgctccaagccagtccaggcgatcgactaccacattgtctacttgccatccccagataatggagaggatgatgaaaattccgccataggcggcataggctcgcccaaagttggcaggttgaagcgttgctacaaatccatatacggtgagtaaaattgctctaactaatgccaaccaaacacttttaccttcccgtatccataaccaaactaagtatccgcccccaatttcacacaaacccgccatgacaaaataaagcagtgatcgcagaatcatcgtccatctccttaatccgattcctgcaaatggtctgcaacttcccgatacaaattcatcacatgattatccgccaagctgtagtaaacattacggccgacccggcgatactttaccaggcgctgcgatcgtaaaattcgtaattgatgggaaactgccgattcactcactttcatcgccgctgctaaatcacagacacagagttcttggcgggccaatgccgacattaaacgcaaccgactcggatcagctagtgcactgaaaaactccgccatttgctgggcctggtccaatgacatcacctctggttgaacctgtcgtacctgctcaagatgaacaagaggttgatcacaaaggggcatctcttcgttctggcaggattgtgactttgacaacgaggacttactcatagaggttggcgttaggagctagggaaaaatttaaactggatttagaaaatgattttcatcctaacatctttaatatctgagcatatcttcaggtgtttcaagatttgtgctacggttcaaggaggtttttctttaaatcacgttggccgccatgacccaatcttcaccgctcaaaactcagcaaatgcaagtcggcggcatggactgcactagttgcaagttaaaaattgaaggtagcttggaaagattgaagggagtagcagaggcctctgtcacggtggccacgggacgattaactgttacctatgatcccaagcaggtcagtgaaataaccattcaagaacggatcgccgccctgggttacacccttgcagagccaaaatcatctgtaacgcttaatggtcataaacatccccactcccatagggaagaaggccatagccatagtcatggtgctggtgaatttaatctgaaacaagaactactgcccgtcctgacggcgatcgccctctttaccattgctattttgttcgagcaaccgttacacaacacgccaggtcagattgctgaatttgcagtgattattcctgcctatctcttgagtggttggacagtgctcaaaacagcgggacgcaatattctcagagggcagatttttgatgaaaactttttaatgacgatcgccaccttgggggccttggcaattcatcaactccccgaagccgtggccgtgatgttgttctttcgagtgggagagctatttcaagaatactccgttgggcgatcgcgccgttccatcaaagctttactggaagcacgtcccgataccgcgaacttgaagcgcaatggtacagtgcaacaagtttcccccgaaacggtacaggttgatgacttaattttggtaaagccgggtgaaaaagtgcccctggatggcgagattttagggggaacttcacaggtagatacttcagcactgactggcgaatcggtgcccggcacagttaagcctggagataccattcttgcaggcatgatcaaccaatcgggtgtgttgacaattcgagtcaccaaactatttagtgaatcttcgatcgccaaagtgctcgatctggttgaaaatgcgtctagcaaaaaggcatccaccgagaaattcatcacccaatttgcccgctactacactcccgttatcgtctttctttcgttagccgttgccctgttaccacctctgttcatccctggggcggatcgagccgattgggtttatcgtgccttggttctgttggtgatttcctgtccctgtggattggtgatcagcattccgctcggctattttggtggcattgggggagccgccaaacatgggattttgatcaagggatctacctttttagactccctcacggccgttaaaaccgttgtgtttgataaaactggaaccttaaccaaaggcacatttaaggttactcaggtcgttaccaaaaatggattttctgaatcagaattgttaaccctagccgcaaaagccgaatcccattccactcatcccattgccctatcgatccgcgaagcttatgcccagtcgatcgctgactctgaagtggccgactacgaagaaattgctgggcatggcattcgagccgttgtgcaaaatcaagtcgtcattgcgggcaatgatcgccttttgcatcgagaaaaaatcgatcatgatacctgtgacgtagccggtacagtggttcatctcgccgtagatgggcgctatgggggctatattctgattgccgacgaaattaaggaagatgcggttcaagcgattcgagacctaaaacgcatgggagttgaaaagaccgtgatgctgaccggggatagtgaaatagtagctcaatccgttgcccagcaaattggtttagatgcctttgttgctgaattattgccagaagaaaaagtcgatgaaattgagcaactgctagatccttcaggtaaagcaaaactggcatttgtaggagatggtattaatgatgcccccgtgattgcccgagccgatgtcggtatagccatggggggattgggctccgatgctgccattgaaacagcggacgtggtgttgatgaccgatgcgccctcgaaggtggccgaagcgattcatgtggcccgcaaaactcgtcaaattgttgtacagaacattgtcctagccctagggattaaagcactttttatcgccctgggcaccattggcttggcaaccctctgggaggcggtgtttgcggatgtgggagtagcactgttagcgattttgaatgctactcggattgctaagtaaagtgcattgacagtatccataccctgttaggtgagatttttggtacactctcgg

SEQ ID NO: 12 - DNA, 12008 bp, Artificial
  plasmid # 1066 pVZhisB-ziaRA-PpsaA**EFEoop standard
tcgacgtcttgactagggggggggaggtggtataatctactagtgaattaaggaggcaatcatatgaccaacttgcagactttcgagttgcctaccgaggtaaccggctgcgccgccgatatctccttgggacgggcgctgatccaagcctggcaaaaagatggcatttttcagatcaagaccgatagtgagcaggatcgcaaaacgcaggaagcaatggctgccagcaagcagttttgcaaggaaccgctgacttttaagagtagctgcgttagcgatctgacctacagcggctatgttgcgtccggcgaggaagtcaccgctggtaaacctgatttccctgaaatcttcactgtctgcaaggacttgtccgtaggcgatcagcgtgtaaaagccggctggccttgccatggtccggtgccatggccaaataacacctatcagaaaagcatgaagaccttcatggaagagctgggtttagcgggcgaacggttgctcaaactgaccgcgctcggctttgaattgcccatcaacacgttcaccgacttaactcgcgatggttggcaccacatgcgtgtattacgcttcccgccccaaacctccacgctgtcccgtggaattggtgcgcacactgactatgggttgttggtaattgccgctcaggacgatgttggtggcttatatattcgccctccagtcgagggagagaagcgtaatcgtaactggttgcctggtgagagctccgcaggcatgtttgagcacgatgaaccttggaccttcgtgacgcccaccccaggcgtgtggaccgttttcccaggtgatatcttgcagttcatgaccggcggccagctgctttccactccgcacaaggttaagctcaatacccgcgaacgtttcgcctgcgcttattttcatgagcctaattttgaagcatccgcctatccgttgttcgagcccagcgccaatgagcgtattcattatggtgagcactttaccaacatgtttatgcgttgctatccagatcggatcaccacccagcggatcaacaaggagaatcgcctggcgcacttggaggacttgaagaagtattccgacacccgcgcgaccggctcctgatagaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggatccgcgctttaaagcctccagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttgatgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaaaacgctcggttgccgccgggcgttttttattctagagtggattgcggttactaagagggtaattaattaatgatggaattaagaacctgtttataaagtctgattctgccccctaaatcccccaatactgggggactttgacttagtttccccccaaacttggtgggccaggggggcttttcaaacacgctctaagccctgttagcatacatcatcactaatacaccaaccaaggcgatcgccgctccaagccagtccaggcgatcgactaccacattgtctacttgccatccccagataatggagaggatgatgaaaattccgccataggcggcataggctcgcccaaagttggcaggttgaagcgttgctacaaatccatatacggtgagtaaaattgctctaactaatgccaaccaaacacttttaccttcccgtatccataaccaaactaagtatccgcccccaatttcacacaaacccgccatgacaaaataaagcagtgatcgcagaatcatcgtccatctccttaatccgattcctgcaaatggtctgcaacttcccgatacaaattcatcacatgattatccgccaagctgtagtaaacattacggccgacccggcgatactttaccaggcgctgcgatcgtaaaattcgtaattgatgggaaactgccgattcactcactttcatcgccgctgctaaatcacagacacagagttcttggcgggccaatgccgacattaaacgcaaccgactcggatcagctagtgcactgaaaaactccgccatttgctgggcctggtccaatgacatcacctctggttgaacctgtcgtacctgctcaagatgaacaagaggttgatcacaaaggggcatctcttcgttctggcaggattgtgactttgacaacgaggacttactcatagaggttggcgttaggagctagggaaaaatttaaactggatttagaaaatgattttcatcctaacatctttaatatctgagcatatcttcaggtgtttcaagatttgtgctacggttcaaggaggtttttctttaaatcacgttggccgccatgacccaatcttcaccgctcaaaactcagcaaatgcaagtcggcggcatggactgcactagttgcaagttaaaaattgaaggtagcttggaaagattgaagggagtagcagaggcctctgtcacggtggccacgggacgattaactgttacctatgatcccaagcaggtcagtgaaataaccattcaagaacggatcgccgccctgggttacacccttgcagagccaaaatcatctgtaacgcttaatggtcataaacatccccactcccatagggaagaaggccatagccatagtcatggtgctggtgaatttaatctgaaacaagaactactgcccgtcctgacggcgatcgccctctttaccattgctattttgttcgagcaaccgttacacaacacgccaggtcagattgctgaatttgcagtgattattcctgcctatctcttgagtggttggacagtgctcaaaacagcgggacgcaatattctcagagggcagatttttgatgaaaactttttaatgacgatcgccaccttgggggccttggcaattcatcaactccccgaagccgtggccgtgatgttgttctttcgagtgggagagctatttcaagaatactccgttgggcgatcgcgccgttccatcaaagctttactggaagcacgtcccgataccgcgaacttgaagcgcaatggtacagtgcaacaagtttcccccgaaacggtacaggttgatgacttaattttggtaaagccgggtgaaaaagtgcccctggatggcgagattttagggggaacttcacaggtagatacttcagcactgactggcgaatcggtgcccggcacagttaagcctggagataccattcttgcaggcatgatcaaccaatcgggtgtgttgacaattcgagtcaccaaactatttagtgaatcttcgatcgccaaagtgctcgatctggttgaaaatgcgtctagcaaaaaggcatccaccgagaaattcatcacccaatttgcccgctactacactcccgttatcgtctttctttcgttagccgttgccctgttaccacctctgttcatccctggggcggatcgagccgattgggtttatcgtgccttggttctgttggtgatttcctgtccctgtggattggtgatcagcattccgctcggctattttggtggcattgggggagccgccaaacatgggattttgatcaagggatctacctttttagactccctcacggccgttaaaaccgttgtgtttgataaaactggaaccttaaccaaaggcacatttaaggttactcaggtcgttaccaaaaatggattttctgaatcagaattgttaaccctagccgcaaaagccgaatcccattccactcatcccattgccctatcgatccgcgaagcttatgcccagtcgatcgctgactctgaagtggccgactacgaagaaattgctgggcatggcattcgagccgttgtgcaaaatcaagtcgtcattgcgggcaatgatcgccttttgcatcgagaaaaaatcgatcatgatacctgtgacgtagccggtacagtggttcatctcgccgtagatgggcgctatgggggctatattctgattgccgacgaaattaaggaagatgcggttcaagcgattcgagacctaaaacgcatgggagttgaaaagaccgtgatgctgaccggggatagtgaaatagtagctcaatccgttgcccagcaaattggtttagatgcctttgttgctgaattattgccagaagaaaaagtcgatgaaattgagcaactgctagatccttcaggtaaagcaaaactggcatttgtaggagatggtattaatgatgcccccgtgattgcccgagccgatgtcggtatagccatggggggattgggctccgatgctgccattgaaacagcggacgtggtgttgatgaccgatgcgccctcgaaggtggccgaagcgattcatgtggcccgcaaaactcgtcaaattgttgtacagaacattgtcctagccctagggattaaagcactttttatcgccctgggcaccattggcttggcaaccctctgggaggcggtgtttgcggatgtgggagtagcactgttagcgattttgaatgctactcggattgctaagtaaagtgcattgacagtatccataccctgttaggtgagatttttggtacactctcgg

SEQ ID NO: 13 - DNA, 11365 bp, Artificial
  plasmid # 1043 pVZhisB-narB-PpsaA**EFEoop standard
tcgacgtcttgactagggggggggaggtggtataatctactagtgaattaaggaggcaatcatatgaccaacttgcagactttcgagttgcctaccgaggtaaccggctgcgccgccgatatctccttgggacgggcgctgatccaagcctggcaaaaagatggcatttttcagatcaagaccgatagtgagcaggatcgcaaaacgcaggaagcaatggctgccagcaagcagttttgcaaggaaccgctgacttttaagagtagctgcgttagcgatctgacctacagcggctatgttgcgtccggcgaggaagtcaccgctggtaaacctgatttccctgaaatcttcactgtctgcaaggacttgtccgtaggcgatcagcgtgtaaaagccggctggccttgccatggtccggtgccatggccaaataacacctatcagaaaagcatgaagaccttcatggaagagctgggtttagcgggcgaacggttgctcaaactgaccgcgctcggctttgaattgcccatcaacacgttcaccgacttaactcgcgatggttggcaccacatgcgtgtattacgcttcccgccccaaacctccacgctgtcccgtggaattggtgcgcacactgactatgggttgttggtaattgccgctcaggacgatgttggtggcttatatattcgccctccagtcgagggagagaagcgtaatcgtaactggttgcctggtgagagctccgcaggcatgtttgagcacgatgaaccttggaccttcgtgacgcccaccccaggcgtgtggaccgttttcccaggtgatatcttgcagttcatgaccggcggccagctgctttccactccgcacaaggttaagctcaatacccgcgaacgtttcgcctgcgcttattttcatgagcctaattttgaagcatccgcctatccgttgttcgagcccagcgccaatgagcgtattcattatggtgagcactttaccaacatgtttatgcgttgctatccagatcggatcaccacccagcggatcaacaaggagaatcgcctggcgcacttggaggacttgaagaagtattccgacacccgcgcgaccggctcctgatagaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgctagaacggatatgctaaatgcgtaaactgcatatcccttggctgagtgtaatttacgttacaaattttaacgaaacgggaaccctatattgatctctacgtttatggaggactgaccatatgaattcactgccaatattttaatggattcaccagctattcttcccaccaccagaaccctttgtccttactgtggagttggttgtggtttggaggctgttgctagtccccaaaagtcggtagttgatgccggtcatgcccataaaattagaggcgatcgccagcatccttccagccaaggcatggtctgcgtcaaaggagctaccgtaatggaatccatggataaacagcgattactttaccccatgtttcggtcaagtttagatcagccgtggcaacaaattagttgggaagcagccctagaaattgttgttgataaaattcagcaagttaaacaaactttgggggtaagtggtttatgtatgtacgcctctgggcaaatgcagacagaggattactacgtggcccaaaaattatttaaaggttgtttgggtaccaataattttgataccaattctcgtctttgcatgtcttcggctgtgtctgcctatagcctaagctttggttccgatggtccgccttgctgttatgaagacttagaaataaccgattgcgcttttttaattggtactaatacagcggactgccatcccattatttttaatcgtttacgcaaacaccacaaacaaaatccccacgttaaattaattgtggtggacccccgttgtactgccaccgcagaagttgcggacttacatttagcgatcaatcctggcagtgacattactttactccatggcattgcctatttgctaaaaaagtggaatttaattgatcaaaaatttattgataatcacacccaagattttgagcagtattgtcaagttattgatcattatcccccggaaaaagttacccaaatttgtggcattagtttagaacaattagaaaccgccgctcactattggggcaatgccaaaacagtgctttccctttggtccatgggaatgaatcaatcttttcaaggcacagctaaaggtcgttgtttaattaatctccatttactcactggtcagattgggaaaccaggcagtggacctttttccctcactggtcagcccaacgccatggggggtcgggaagcagggggattgtcccatctgttgcctggctatcgttcaattaaaaatcctcaacaccgccaagaggtagaacaactttggcagatttcccctggccgtattaatccagaaccaggtttatcagcctgggaaatgttcatgggattggaaaatcaacaggttggtttcctatggattgtggcgactaaccctgttgttagtatgcctgatttagaaagggttaaaaaggctctgcaacaatcaacttttacgattcatcaggatgcctattcccccactgaaacggcagaatacgcccatttgttattgccagcagctcaatggagcgaaaaaactggcacaatgaccaattcagaacggagagttaccctttctcctgcgtttcgctctcctcctggggaagcaagacccgactgggaaatttttgctgaggtaggacgtagattaggttttgaaaatcaatttaattttgttgattcagccgcagtacatcgggaatatgtgcaactaactgctgaaagattatgtgaccaatctggtgtcagttatgaaaagttgcaaaagctggggccgctgcaatggccctgtcgtcaatcagatcaagaaagtcaacttttatcaactaaaagactttacaccgactataaattttgcacagaaaatggtcgggcaaacttttgcttagaccacagtcggggattagctgagccggtcgatccaaactatccttttgtcctcaccaatggtcgtctttatggtcattggcatacccaaacccgcactggccacattgagaaaatcaaaaaaatgcatcccaaacctatattggaaatgcatcccaaggacgcagaaaaattgggtattaaaagtcaggatttagttgcgattaaatctcgacggggttctgctcagctagaagttctagttactagagccattgcccccggtacggtttttatgcccatgcactggggttttttgtgggacgataatgctgaagttaatagtttgacccatgccactgcttgccctatttctaaacaaccggaattaaaagcctgcgccgttaatattacaccagtttaatcaaacaataagcaattaattgtcaggataaaaatctaagcttaaaatttggtcaggaatatatgagtattgttgaggaaatcgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgacacaacctaagacttccttccaaaaatccatagggcggtggaagcttagctatttttaccattttgttttgccactcaaatatttacttaaggtgaggtaaaaactcatcttttttttactaaaaattgcggctagaaatgtaatttcggcaatccccccaccttctttcctgaaaaccgaatctaacctggaaggggaaattttaagatagaaccattcaagggtaatcaattccttccacacaggatccgcgctttaaagcctccagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttgatgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaaaacgctcggttgccgccgggcgttttttattctagag

SEQ ID NO: 14 - DNA, 8915 bp, Artificial
  plasmid #818 pJet-corRT(SacB/Gm) standard
catggcagctgagaatattgtaggagatcttctagaaagattctagatcaattttctaccccaaacccccacaatgggcaaactaccgcctatctttcactgcttgcggaaccgtctatttgctcagctatacctagcccaagccattagtttgctgggggatgccctgacctgggtaggattagcacttttagcttttgaattggcgggtcaaggggcggggttgatcctggcaggggccttaaccttaagggtaacggtgtttgtcttgctgtctcccattgcgggggcgatcgccgaccgttatgatcgcaagcaaatgatggtgattacccatctggcacggctggggattgtctgtttatttcctggggtgacccaggcctggcaaatctacgggctggttctggggctgaatgtgtttaacgccttttttactccgacctataccgcaaccattcccctggtgacgaaggaggatgaatatccccaggcgatcgccttatccagtgccacttatcaacttttgggggttttggggccagggttggctgggagtcttgccgcttgggtgggaacaaaaacaatcttttggggggatgctttaacttttttgatggccgctggtttaatttttaccttgccaggaaaactgttagctaattcaaccgcccagcctgtccgtaacttggcccaaattcgacgggacatcggcacaggaacccaatgtcttttcggcgatcgcctgattcgttatgctctagcgatgcagttggtggtgtccttggccggggcagggattttagtgaatacggtgggctatgtccaaggcattttaaacctgggtaagctggagtatggttggctaatggcggcgtttgggttgggagcgacggtggcttccctcggtttgggcaacactcagcaacagagaaaacgaatctatttaacgacaattggggctgtagtaatgagtcttgcgattctacccgtctcgatggtcaatcttcagggattattgctcctgtgggctggggctggtatcggccaaaccttggttaatgtccccacccaaaccctgattgccgaccgcgtagccaaagagcttcaaggccgagtgtatggtgccaattttgcctggagtcatctctggtgggcgttttcctatcctctggcgggatggttaggcagtcactttgctcaaaatagttttttctatctggggattttagccctcagcttatttgccctgttttatctgttacgaccctccactccgatggaaccaggattttggcacgaacattcccatcaccatgactccgagcatagtcattttcatcctgcgacaactgtgctgaattttagccacaatcatcttcatttccatttttataaggcgagtccttaaactacccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatgctcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccaattcgagctcggtctcgacggtatcgataagcttgatatcgaattcctgcagccaagctagacctaggccttaagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggaggtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatatgaactatataaaagcaggcaaatggctaaccgtattcctaaccttttggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagcctgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcaggccggcattaatatcttgctgaaaaactcgagcttttcaaacacgctctaagccctgttagcatacatcatcactaatacaccaaccaaggcgatcgccgctccaagccagtccaggcgatcgactaccacattgtctacttgccatccccagataatggagaggatgatgaaaattccgccataggcggcataggctcgcccaaagttggcaggttgaagcgttgctacaaatccatatacggtgagtaaaattgctctaactaatgccaaccaaacacttttaccttcccgtatccataaccaaactaagtatccgcccccaatttcacacaaacccgccatgacaaaataaagcagtgatcgcagaatcatcgtccatctccttaatccgattcctgcaaatggtctgcaacttcccgatacaaattcatcacatgattatccgccaagctgtagtaaacattacggccgacccggcgatactttaccaggcgctgcgatcgtaaaattcgtaattgatgggaaactgccgattcactcactttcatcgccgctgctaaatcacagacacagagttcttggcgggccaatgccgacattaaacgcaaccgactcggatcagctagtgcactgaaaaactccgccatttgctgggcctggtccaatgacatcacctctggttgaacctgtcgtacctgctcaagatgaacaagaggttgatcacaaaggggcatctcttcgttctggcaggattgtgactttgacaacgaggacttactcatagaggttggcgttaggagctagggaaaaatttaaactggatttagaaaatgattttcatcctaacatctttaatatctgagcatatcttcaggtgtttcaagatttgtgctacggttcaaggaggttgcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttc

SEQ ID NO: 15 - DNA, 5224 bp, Artificial
  plasmid # 822 pJet-delta\corRT standard
catggcagctgagaatattgtaggagatcttctagaaagattctagatcaattttctaccccaaacccccacaatgggcaaactaccgcctatctttcactgcttgcggaaccgtctatttgctcagctatacctagcccaagccattagtttgctgggggatgccctgacctgggtaggattagcacttttagcttttgaattggcgggtcaaggggcggggttgatcctggcaggggccttaaccttaagggtaacggtgtttgtcttgctgtctcccattgcgggggcgatcgccgaccgttatgatcgcaagcaaatgatggtgattacccatctggcacggctggggattgtctgtttatttcctggggtgacccaggcctggcaaatctacgggctggttctggggctgaatgtgtttaacgccttttttactccgacctataccgcaaccattcccctggtgacgaaggaggatgaatatccccaggcgatcgccttatccagtgccacttatcaacttttgggggttttggggccagggttggctgggagtcttgccgcttgggtgggaacaaaaacaatcttttggggggatgctttaacttttttgatggccgctggtttaatttttaccttgccaggaaaactgttagctaattcaaccgcccagcctgtccgtaacttggcccaaattcgacgggacatcggcacaggaacccaatgtcttttcggcgatcgcctgattcgttatgctctagcgatgcagttggtggtgtccttggccggggcagggattttagtgaatacggtgggctatgtccaaggcattttaaacctgggtaagctggagtatggttggctaatggcggcgtttgggttgggagcgacggtggcttccctcggtttgggcaacactcagcaacagagaaaacgaatctatttaacgacaattggggctgtagtaatgagtcttgcgattctacccgtctcgatggtcaatcttcagggattattgctcctgtgggctggggctggtatcggccaaaccttggttaatgtccccacccaaaccctgattgccgaccgcgtagccaaagagcttcaaggccgagtgtatggtgccaattttgcctggagtcatctctggtgggcgttttcctatcctctggcgggatggttaggcagtcactttgctcaaaatagttttttctatctggggattttagccctcagcttatttgccctgttttatctgttacgaccctccactccgatggaaccaggattttggcacgaacattcccatcaccatgactccgagcatagtcattttcatcctgcgacaactgtgctgaattttagccacaatcatcttcatttccatttttataaggcgagtccttaaactacccggcattaatatcttgctgaaaaactcgagcttttcaaacacgctctaagccctgttagcatacatcatcactaatacaccaaccaaggcgatcgccgctccaagccagtccaggcgatcgactaccacattgtctacttgccatccccagataatggagaggatgatgaaaattccgccataggcggcataggctcgcccaaagttggcaggttgaagcgttgctacaaatccatatacggtgagtaaaattgctctaactaatgccaaccaaacacttttaccttcccgtatccataaccaaactaagtatccgcccccaatttcacacaaacccgccatgacaaaataaagcagtgatcgcagaatcatcgtccatctccttaatccgattcctgcaaatggtctgcaacttcccgatacaaattcatcacatgattatccgccaagctgtagtaaacattacggccgacccggcgatactttaccaggcgctgcgatcgtaaaattcgtaattgatgggaaactgccgattcactcactttcatcgccgctgctaaatcacagacacagagttcttggcgggccaatgccgacattaaacgcaaccgactcggatcagctagtgcactgaaaaactccgccatttgctgggcctggtccaatgacatcacctctggttgaacctgtcgtacctgctcaagatgaacaagaggttgatcacaaaggggcatctcttcgttctggcaggattgtgactttgacaacgaggacttactcatagaggttggcgttaggagctagggaaaaatttaaactggatttagaaaatgattttcatcctaacatctttaatatctgagcatatcttcaggtgtttcaagatttgtgctacggttcaaggaggttgcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttc

SEQ ID NO: 16 - DNA, 14079 bp, Artificial
  plasmid # 861 pVZ-hisB-corRT-PpetJ-PDC-Prbc*-synADH(deg)
tcgacgggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcaactttgacagattgtcttttgactttgcctggaccgcctcccataataccttcgcgtcttgaagactttatccttgaaaggagaactaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctcggtacccaaactagtcgatcgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggatccgcgctttaaagcctccagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttgatgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaaaacgctcggttgccgccgggcgttttttattctagaaagataggcgagtccttaaactacccgcttaaaaaccatgcgtccaaaactttcaccatcctttccctatcaacctttactgcactaaagacaagtgagatagcagtggcaatctggctttgcaatcaatgtttccactaaagcgtttagcgttactgcggctagaagtcctccaccgaggctcccctgaatggtgatatggggaatgggactggtcatcagtcgtcgttttgcccccggagcatgactaaaaccgatcggcattccgatcacaagagccggctgaatatgttgttgctctatcagcttacaggcagtgagtaaaacagaaggggcatagccgatcgccagcacacatccttggggaatctgttgtaaccgctgttgccaatggtcatggtgccaaaaagcttgctcggcttccctaagccctgtgatgtgagggtcgtcaatcagcgttttaaccgtacatcctaaatgagctaaccgagtttgatcaagagccgcagccacaaccggaacatcggtgacgactggacaccctgctttcagtgcatctcgtgccgaggcgatcgctccctgactcaatcgaacggcgtttaccaagctaacatcaccacaggccagcactaattgatgtagtaagtgaatggtaatttcagagtaagccgataaatccggtagcaggtgtttgagggattcctgaaaggcttctggatgagttgttgtctccgcatctaggttcgtccacaactgatcgagttttcctaacccctcctggacatccacatcaagctgtttcagttgggccagagcttccgcttgggtaatctggcaactctggtcgcgtcccagtaatccttctaaagcagatgcggtttggcggagtcgagtaatctgctgaatcacagcctgatattgctgttgcaactgcaccattagggtgggatcaaggctctcttcagaatggctatccagcagttgccgaatatgagacaactgaaagccctgctgtttgagggcaatgactcgttggagccgttgtacgtcctgctgagtataaaggcggtagttgccctctgagcgttgaacggggggaagcaatcccagggtgtggtaatggcgcaccatgcgaggcgtaacgccacctcccactgcatctgtgagttctttaatcgttaagtgattagtcttcatccctttagtttactcaaaaccttgacattgacactaatgttaaggtttaggctgagaaggtaaaaatccaagttaaaaagcatggttgtaactcccccttcttctgcctttcgattttctaaccttttcaaagatcatcccgatgcagtggcggcgatcgcctgtggcgggctggtctttctgggctggcagatgcttaacttgggctggttgggaattgccttttttgtcctaacagcggcctatgtgattggtggatttgacaatgcccgtgagggattaacaaccctctttgaagaaaaagaatttgatgtcgatctgctgatgattgtggccgccttgggggccgccgggttgggactttggcgacgggaatataccctcattgtggatggggcagttctgattcttatttttgccatcagtggggcattggaaggatacgcgatgcaacggacggagcggagtatccagggattgatgagcttaaccgctgatgtagcaagagtgttgcggaacggacaggagcaaaccattcccatttcagaactgaagatgggggatcaagtcttggtgaaaccgggagagttggtgccaacggatggcttggtcattgaagggtttagcaccctgaaccaggcttccatcacgggggaatctatgccagtggaaaaggcgatcggcgatgaggtgtttgccggcacgatcaatggcaatggggtactgcggctcaaaatccatcaacccccggaaagtagcttgattcagcgtgtgattcgtttggttcagcaagcccaaaccgaagctcccccgtcgcaacagtttattgaacgatttgagtgcggttatgccaaggtaattgtgatcgccggactcttgctcggtacgttgccgccttttttgctgggctggagttgggaagagacgatctaccgtgccttaatttttctcgtggtggcttctccctgtgcgctaatggcatcaattatgccggccttactttctggtattgccaatggagcgcgtcaaggcattttattcaaaaacggggcgcagttggagcggattggacgagtacgggtgattgcttttgataaaactggaacattgaccactggaaaacctgaagttgtgaacattctcgccactcagccatccaccgacaaacttctgcaaatcgcggcggccctggaaagtttatcggaacatcccatcggagaggcgatcgccgacttcacccgtcaacagaaccaagcctgggcaacagccaggaacgttcaagcgcaggcgggacagggcattatcggagacattgagggccaacaggcgatagttggtaaagccgtgtttgttcaggcccaggtcaatcatgttgctacaaacctgatagagcaaagtcagcagtgggaagctgaaggtaaaaccgtcgtctgggttgcctacgctggagagattttaggtctcattgccgtggcagatactgtacgacccaccgccgcccaggcgatcgcccgattaaaacgattaggaattgagcgaattgtcatgctaactggggataactcacgtaccgcccacagcattgctcaacaggtgggagtcaatcaggtttatgcagaacttttgcccgaagataaagtggatgtgattcgccaactccagaagcagtatcaatccgtcgccatggtgggagacggcatcaatgatgcacccgccttagctcaggcatctgttggtattgcgatgggggccgctggtagtgatgtggccttagaaaccgcagatattgtcttaatggcggatcgtttggagcggttagaacacgccattcgtttgggtcgtcgcgctcaaggagtggtcaaacaaaacattgtgtttgcccttggttttgtgatgattctgttgattgcaaactttgcgggtaatatcacgctgccctttggggttttggggcatgaaggctcgacggtaatcgtaacgttgagtggattgcggttactaagagggtaattaattaatgatggaattaagaacctgtttataaagtctgattctgccccctaaatcccccaatactgggggactttgacttagtttccccccaaacttggtgggccaggggggcttttcaaacacgcg

SEQ ID NO: 17 - DNA, 14301 bp, Artificial
  plasmid # 870 pVZ-hisB-corRT-PpetJ-PDCoop-PrbcL-synADH(deg)
tcgacgggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcaactttgacagattgtcttttgactttgcctggaccgcctcccataataccttcgcgtcttgaagactttatccttgaaaggagaactaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctcggtacccaaactagtaacgctcggttgccgccgggcgttttttattccgacatcaggaattgtaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactgacccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggatccgcgctttaaagcctccagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttgatgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaaaacgctcggttgccgccgggcgttttttattctagaaagataggcgagtccttaaactacccgcttaaaaaccatgcgtccaaaactttcaccatcctttccctatcaacctttactgcactaaagacaagtgagatagcagtggcaatctggctttgcaatcaatgtttccactaaagcgtttagcgttactgcggctagaagtcctccaccgaggctcccctgaatggtgatatggggaatgggactggtcatcagtcgtcgttttgcccccggagcatgactaaaaccgatcggcattccgatcacaagagccggctgaatatgttgttgctctatcagcttacaggcagtgagtaaaacagaaggggcatagccgatcgccagcacacatccttggggaatctgttgtaaccgctgttgccaatggtcatggtgccaaaaagcttgctcggcttccctaagccctgtgatgtgagggtcgtcaatcagcgttttaaccgtacatcctaaatgagctaaccgagtttgatcaagagccgcagccacaaccggaacatcggtgacgactggacaccctgctttcagtgcatctcgtgccgaggcgatcgctccctgactcaatcgaacggcgtttaccaagctaacatcaccacaggccagcactaattgatgtagtaagtgaatggtaatttcagagtaagccgataaatccggtagcaggtgtttgagggattcctgaaaggcttctggatgagttgttgtctccgcatctaggttcgtccacaactgatcgagttttcctaacccctcctggacatccacatcaagctgtttcagttgggccagagcttccgcttgggtaatctggcaactctggtcgcgtcccagtaatccttctaaagcagatgcggtttggcggagtcgagtaatctgctgaatcacagcctgatattgctgttgcaactgcaccattagggtgggatcaaggctctcttcagaatggctatccagcagttgccgaatatgagacaactgaaagccctgctgtttgagggcaatgactcgttggagccgttgtacgtcctgctgagtataaaggcggtagttgccctctgagcgttgaacggggggaagcaatcccagggtgtggtaatggcgcaccatgcgaggcgtaacgccacctcccactgcatctgtgagttctttaatcgttaagtgattagtcttcatccctttagtttactcaaaaccttgacattgacactaatgttaaggtttaggctgagaaggtaaaaatccaagttaaaaagcatggttgtaactcccccttcttctgcctttcgattttctaaccttttcaaagatcatcccgatgcagtggcggcgatcgcctgtggcgggctggtctttctgggctggcagatgcttaacttgggctggttgggaattgccttttttgtcctaacagcggcctatgtgattggtggatttgacaatgcccgtgagggattaacaaccctctttgaagaaaaagaatttgatgtcgatctgctgatgattgtggccgccttgggggccgccgggttgggactttggcgacgggaatataccctcattgtggatggggcagttctgattcttatttttgccatcagtggggcattggaaggatacgcgatgcaacggacggagcggagtatccagggattgatgagcttaaccgctgatgtagcaagagtgttgcggaacggacaggagcaaaccattcccatttcagaactgaagatgggggatcaagtcttggtgaaaccgggagagttggtgccaacggatggcttggtcattgaagggtttagcaccctgaaccaggcttccatcacgggggaatctatgccagtggaaaaggcgatcggcgatgaggtgtttgccggcacgatcaatggcaatggggtactgcggctcaaaatccatcaacccccggaaagtagcttgattcagcgtgtgattcgtttggttcagcaagcccaaaccgaagctcccccgtcgcaacagtttattgaacgatttgagtgcggttatgccaaggtaattgtgatcgccggactcttgctcggtacgttgccgccttttttgctgggctggagttgggaagagacgatctaccgtgccttaatttttctcgtggtggcttctccctgtgcgctaatggcatcaattatgccggccttactttctggtattgccaatggagcgcgtcaaggcattttattcaaaaacggggcgcagttggagcggattggacgagtacgggtgattgcttttgataaaactggaacattgaccactggaaaacctgaagttgtgaacattctcgccactcagccatccaccgacaaacttctgcaaatcgcggcggccctggaaagtttatcggaacatcccatcggagaggcgatcgccgacttcacccgtcaacagaaccaagcctgggcaacagccaggaacgttcaagcgcaggcgggacagggcattatcggagacattgagggccaacaggcgatagttggtaaagccgtgtttgttcaggcccaggtcaatcatgttgctacaaacctgatagagcaaagtcagcagtgggaagctgaaggtaaaaccgtcgtctgggttgcctacgctggagagattttaggtctcattgccgtggcagatactgtacgacccaccgccgcccaggcgatcgcccgattaaaacgattaggaattgagcgaattgtcatgctaactggggataactcacgtaccgcccacagcattgctcaacaggtgggagtcaatcaggtttatgcagaacttttgcccgaagataaagtggatgtgattcgccaactccagaagcagtatcaatccgtcgccatggtgggagacggcatcaatgatgcacccgccttagctcaggcatctgttggtattgcgatgggggccgctggtagtgatgtggccttagaaaccgcagatattgtcttaatggcggatcgtttggagcggttagaacacgccattcgtttgggtcgtcgcgctcaaggagtggtcaaacaaaacattgtgtttgcccttggttttgtgatgattctgttgattgcaaactttgcgggtaatatcacgctgccctttggggttttggggcatgaaggctcgacggtaatcgtaacgttgagtggattgcggttactaagagggtaattaattaatgatggaattaagaacctgtttataaagtctgattctgccccctaaatcccccaatactgggggactttgacttagtttccccccaaacttggtgggccaggggggcttttcaaacacgcg

SEQ ID NO: 18 - DNA, 8716 bp, Artificial
  plasmid # 1160 pJet-smtA/B(SacB/Gm) standard
catggcagctgagaatattgtaggagatcttctagattgaccgccgattcagtggatgaattggtattccagggcaatgaatattatgaccaagggcaatatgaactggccctcgcagcctacgatgccgccttaattttgaatgcgaacgatgcagagctttggatctggcggggcgttaccctaggggccttggggcagcacgaagaggaattggccagctatgatcaggcgatcgcccttgaccccgatcaaaacctggtgtggtacaaccggggcgttgtgttaggagctttagagcgctacgaagaagaactggccagctatgaccgcgccctggaactggatccgggggatgtggatgcttggtataaccggggtattgccctgggctttctggaacgctatgaagaagaattagccagctacgatcaagtactagcgttagatcccgcagatgtggatgcttggttcaataaaggctatgttctggccgatttaggtcgccatgaagaagcgatcgccacctatgccgccctcctagaactcgatcccgacgacgccgaagcctggaataatcacggtatttccctcgaagcactggggcgctataccgaggcgatcgccagctatgaccaagccctcgccattgacccagactacgaagacgctttgattaatcgagaaattgcccaagctgccctagaacaactttaaacttcaactaaatcaatctttatcaaaaaaaatacccagcttcggtgatgggtattgcttcagcttataaatttggctcaatttctttctattgatggccccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatgctcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccaattcgagctcggtctcgacggtatcgataagcttgatatcgaattcctgcagccaagctagacctaggccttaagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggaggtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatatgaactatataaaagcaggcaaatggctaaccgtattcctaaccttttggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagcctgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcaggccggcattaatatcttgctgaaaaactcgagctatcgcaaggaagggcggaatgtgatttatcgcctcaaggatcaccacattcttaatctctatcgtgacgcttcggagcacctagacgaaccagaggatggacacacccactaatcaacgcggctgccggttgcttggcggatataggcttgatctgcttcataaagtttgcccatggctggattgtggggcggtaaacccaaaatggcaagatctgcttcgaccatgatcccgacgagggtcggaaaatcaatactcggctcccagtttagttgttttttagccttcgtgggatcgccaatgagtaagtccacctcggcagggcggagataacggcggtcaaaggcgacataatcttcccagttcagattgacgtaggcgaaggcggtttctaaaaattggcggactgaataggtttcccctgtggcaatgacatagtcgtcgggtttgtcctgctgcaacatcagccacattgcccgcacatagtcttttgcataaccccagtcccgcttggaatcgaggttgccgagatacagtttgtcctgctgtccggcgataattctggcgatcgccctggtgatttttcgggtaacgaaggtttccccccggcgcggcccttcatggttaaacaaaatgccattgcaagcgaacaaatcataggattctcggtagttcaccgtttgccagtagccatagaccttggcacaggcgtagggactgcggggataaaagggggtcgtttccttttggggcacctcttgcacaaggccaaacatttccgacgaaccggcctggtaaaagcgcacttcattgcccgtccgttcctggtagtcccgaatcgcttctaggatacgcagcgtccccatcgccacggtatccacggtatattctggcgaatcaaagctcacccgcacatgggattgggcacccagattgtaaacttcatggggctggattttttcgattaattttcctagggatgtgccatcggtgagatccccgtaatggagaaagaaccgagcgtcggggatatggggatcgacatagagatggtcaatgcggtcggtattaaaagtagaagcgcgacggataatgccatgaacttcgtaacctttttcgaggagcagttccgccaaataagagccatcttgtccggtgatccctgttaccagtgcttttcgattacccatggagctcgtgtttactcaacatatcaactaagcaattttagaacagtattcaccgagatggactttaagacgccatttacatggtgccgcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttc

SEQ ID NO: 19 - DNA, 5025 bp, Artificial
  plasmid # 1228 pJet-delta\smtA/B standard
catggcagctgagaatattgtaggagatcttctagattgaccgccgattcagtggatgaattggtattccagggcaatgaatattatgaccaagggcaatatgaactggccctcgcagcctacgatgccgccttaattttgaatgcgaacgatgcagagctttggatctggcggggcgttaccctaggggccttggggcagcacgaagaggaattggccagctatgatcaggcgatcgcccttgaccccgatcaaaacctggtgtggtacaaccggggcgttgtgttaggagctttagagcgctacgaagaagaactggccagctatgaccgcgccctggaactggatccgggggatgtggatgcttggtataaccggggtattgccctgggctttctggaacgctatgaagaagaattagccagctacgatcaagtactagcgttagatcccgcagatgtggatgcttggttcaataaaggctatgttctggccgatttaggtcgccatgaagaagcgatcgccacctatgccgccctcctagaactcgatcccgacgacgccgaagcctggaataatcacggtatttccctcgaagcactggggcgctataccgaggcgatcgccagctatgaccaagccctcgccattgacccagactacgaagacgctttgattaatcgagaaattgcccaagctgccctagaacaactttaaacttcaactaaatcaatctttatcaaaaaaaatacccagcttcggtgatgggtattgcttcagcttataaatttggctcaatttctttctattgatggccccggcattaatatcttgctgaaaaactcgagctatcgcaaggaagggcggaatgtgatttatcgcctcaaggatcaccacattcttaatctctatcgtgacgcttcggagcacctagacgaaccagaggatggacacacccactaatcaacgcggctgccggttgcttggcggatataggcttgatctgcttcataaagtttgcccatggctggattgtggggcggtaaacccaaaatggcaagatctgcttcgaccatgatcccgacgagggtcggaaaatcaatactcggctcccagtttagttgttttttagccttcgtgggatcgccaatgagtaagtccacctcggcagggcggagataacggcggtcaaaggcgacataatcttcccagttcagattgacgtaggcgaaggcggtttctaaaaattggcggactgaataggtttcccctgtggcaatgacatagtcgtcgggtttgtcctgctgcaacatcagccacattgcccgcacatagtcttttgcataaccccagtcccgcttggaatcgaggttgccgagatacagtttgtcctgctgtccggcgataattctggcgatcgccctggtgatttttcgggtaacgaaggtttccccccggcgcggcccttcatggttaaacaaaatgccattgcaagcgaacaaatcataggattctcggtagttcaccgtttgccagtagccatagaccttggcacaggcgtagggactgcggggataaaagggggtcgtttccttttggggcacctcttgcacaaggccaaacatttccgacgaaccggcctggtaaaagcgcacttcattgcccgtccgttcctggtagtcccgaatcgcttctaggatacgcagcgtccccatcgccacggtatccacggtatattctggcgaatcaaagctcacccgcacatgggattgggcacccagattgtaaacttcatggggctggattttttcgattaattttcctagggatgtgccatcggtgagatccccgtaatggagaaagaaccgagcgtcggggatatggggatcgacatagagatggtcaatgcggtcggtattaaaagtagaagcgcgacggataatgccatgaacttcgtaacctttttcgaggagcagttccgccaaataagagccatcttgtccggtgatccctgttaccagtgcttttcgattacccatggagctcgtgtttactcaacatatcaactaagcaattttagaacagtattcaccgagatggactttaagacgccatttacatggtgccgcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttc

SEQ ID NO: 20 - DNA, 14007 bp, Artificial
  plasmid # 1326 pVZ-hisB-PhspA-ziaA-Pind(7002)
tcgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctcagcaagtttcatcccgaccccctcagggtcgggatttttttattgtactagtaacgcccggttgccaccgggcgttttttattccgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtgcgctttaaagcctccagcgcagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttgatgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaaaacgctcggttgccgccgggcgttttttattctagacacggcccggtctcgacaacggggaggggcaggattaggccttgccattgcccaggcgatcgccgttaagcatcaaggtgttttgactgtagagagtgaattgggtcagggaagtttattcaccattcgcttatcaatatgtccgccaccataccaacagtcccgagagtgtaccaaaaatctcacctaacagggtatggatactgtcaatgcactttacttagcaatccgagtagcattcaaaatcgctaacagtgctactcccacatccgcaaacaccgcctcccagagggttgccaagccaatggtgcccagggcgataaaaagtgctttaatccctagggctaggacaatgttctgtacaacaatttgacgagttttgcgggccacatgaatcgcttcggccaccttcgagggcgcatcggtcatcaacaccacgtccgctgtttcaatggcagcatcggagcccaatccccccatggctataccgacatcggctcgggcaatcacgggggcatcattaataccatctcctacaaatgccagttttgctttacctgaaggatctagcagttgctcaatttcatcgactttttcttctggcaataattcagcaacaaaggcatctaaaccaatttgctgggcaacggattgagctactatttcactatccccggtcagcatcacggtcttttcaactcccatgcgttttaggtctcgaatcgcttgaaccgcatcttccttaatttcgtcggcaatcagaatatagcccccatagcgcccatctacggcgagatgaaccactgtaccggctacgtcacaggtatcatgatcgattttttctcgatgcaaaaggcgatcattgcccgcaatgacgacttgattttgcacaacggctcgaatgccatgcccagcaatttcttcgtagtcggccacttcagagtcagcgatcgactgggcataagcttcgcggatcgatagggcaatgggatgagtggaatgggattcggcttttgcggctagggttaacaattctgattcagaaaatccatttttggtaacgacctgagtaaccttaaatgtgcctttggttaaggttccagttttatcaaacacaacggttttaacggccgtgagggagtctaaaaaggtagatcccttgatcaaaatcccatgtttggcggctcccccaatgccaccaaaatagccgagcggaatgctgatcaccaatccacagggacaggaaatcaccaacagaaccaaggcacgataaacccaatcggctcgatccgccccagggatgaacagaggtggtaacagggcaacggctaacgaaagaaagacgataacgggagtgtagtagcgggcaaattgggtgatgaatttctcggtggatgcctttttgctagacgcattttcaaccagatcgagcactttggcgatcgaagattcactaaatagtttggtgactcgaattgtcaacacacccgattggttgatcatgcctgcaagaatggtatctccaggcttaactgtgccgggcaccgattcgccagtcagtgctgaagtatctacctgtgaagttccccctaaaatctcgccatccaggggcactttttcacccggctttaccaaaattaagtcatcaacctgtaccgtttcgggggaaacttgttgcactgtaccattgcgcttcaagttcgcggtatcgggacgtgcttccagtaaagctttgatggaacggcgcgatcgcccaacggagtattcttgaaatagctctcccactcgaaagaacaacatcacggccacggcttcggggagttgatgaattgccaaggcccccaaggtggcgatcgtcattaaaaagttttcatcaaaaatctgccctctgagaatattgcgtcccgctgttttgagcactgtccaaccactcaagagataggcaggaataatcactgcaaattcagcaatctgacctggcgtgttgtgtaacggttgctcgaacaaaatagcaatggtaaagagggcgatcgccgtcaggacgggcagtagttcttgtttcagattaaattcaccagcaccatgactatggctatggccttcttccctatgggagtggggatgtttatgaccattaagcgttacagatgattttggctctgcaagggtgtaacccagggcggcgatccgttcttgaatggttatttcactgacctgcttgggatcataggtaacagttaatcgtcccgtggccaccgtgacagaggcctctgctactcccttcaatctttccaagctaccttcaatttttaacttgcaactagtgcagtccatgccgccgacttgcatttgctgagttttgagcggtgaagattgggtcatatgttaactcctgatgtgtggaaggaattgattacccttgaatggttctatcttaaaatttccccttccaggttagattcggttttcaggaaagaaggtggggggattgccgaaattacatttctagccgcaatttttagtaaaaaaaagatgagtttttacctcaccttaagtaaatatttgagtggcaaaacaaaatggtaaaaatagctaagcttccaccgccctatggatttttggaaggaagtcttaggttgtg

SEQ ID NO: 21 - DNA, 12022 bp, Artificial
  plasmid # 1454 pVZ-hisB-smtAB-Pind(7002)-PDCdsrA-Prbc*-synADH(deg) standard
tcgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctcagcaagtttcatcccgaccccctcagggtcgggatttttttattgtactagtaacgcccggttgccaccgggcgttttttattccgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtgcgctttaaagcctccagcgcagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttgatgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaaaacgctcggttgccgccgggcgttttttattctagatctattggtgacaagtacagcccccatggccacagccttcactccctgcgggatggccatcggcacaggcttggctgcaataactcttaccttccttttggatagcgctattgagatccacgatgcaaagacaagattcacaggcgcacttcatttgggtcacagttaccatggtttaaacctccaaggtattgtcttaaatagttgaacgattgttcacatttttagattaacacatgaacaattattcaggtattgtttagatcagcgaacaatgacagaccacagtggcaaaatcttgaaccaagccaaggcccaacggatggccgaattttttggtgtcttgggggatgccaaccgctggcgtattctttcggccctggccactggagagatgcgggtgggggaactcgccgccgccgttgaaatgagtgaatcggctgtctctcaccaactaagaaccctgcgcacagcacgtttagtgagctatcgcaaggaagggcggaatgtgatttatcgcctcaaggatcaccacattcttaatctctatcgtgacgcttcggagcacctagacgaaccagaggatggacacacccactaatcaacgcggctgccggttgcttggcggatataggcttgatctgcttcataaagtttgcccg

SEQ ID NO: 22 - DNA, 11897 bp, Artificial
  plasmid   #1484 pAQ3::PhspA-ziaA-Pind(7002)-PDCdsrA-Prbc*-synADH(deg)
tcgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctcagcaagtttcatcccgaccccctcagggtcgggatttttttattgtactagtaacgcccggttgccaccgggcgttttttattccgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgccgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgatccccccctcgttcaaggcaggcaacgcccccggcgatcgccgtccttttttatgccacatcttcggtatataaatccgcctgaaaatctgcgaatacttgaccgatatcctgacccaagatcactaaaccttcattaacggtttggtatttgatttcgatgagggtaggcagtttcccccgatcaagttcctccactccttctcgaatgtattggtctaggacaaaatctaaaaattcttgctgctttccggtgtacttcgagaaaatcagatctcgatgcttaattactcgctcttctctgctaatgggtttggtgttgtaggcaacccaagtcaggacatcatagacatcacttttttccgcttcggcaatgcgtgcgatcgccttcagttgggtgtcaccgtagcctttttccgcgagtccggtcaggaacgatttacgggtatcgggtttcccccagatggtgcgtagttcggcttcatccttgaagaggtcgggcaggtcgccaaatagcttttcgataaattcttgggcggaaatgggtttaccatcagcatcccaaaaagttgtggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatcactagtttggagataatcgcctttgggcagttatctagaatgtacacaaatttattaccatctaagacacgacaaaaaatacatactcatttccaattacctaacaagttttttatatcttggttacggtctttgtcgcatattagaaatatttgcgctcatcattcaaggctctggaatatacagctaggagaatcacctaaattgcctgataggttaaaaggaaaatggctctctagagaagtattggaggatattaatcaaagaaacagccgtaaaattttcactggcttatgttgtattcagtatctcttagacagaattaaaccagagcataattttgcacagcatttaaaaagaacttttgagatgtatccagaaatagaatctaaaaacttaggctttcccaaagattgggaaaatcagcctctctggaaataatctaagagtcagaattttaatttgtcataactctttctcgttcaaggcagggcggcctgcacatactgggaagcatattcttcgatgcgcttaaagttttgccgtggtagtttagcttgatgctcttccacgttgaaacctgctaagtagttacatacggctgacagcggcaaaaaatgtttgagtataaggccatagttgatgcttgttggaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgctagacacggcccggtctcgacaacggggaggggcaggattaggccttgccattgcccaggcgatcgccgttaagcatcaaggtgttttgactgtagagagtgaattgggtcagggaagtttattcaccattcgcttatcaatatgtccgccaccataccaacagtcccgagagtgtaccaaaaatctcacctaacagggtatggatactgtcaatgcactttacttagcaatccgagtagcattcaaaatcgctaacagtgctactcccacatccgcaaacaccgcctcccagagggttgccaagccaatggtgcccagggcgataaaaagtgctttaatccctagggctaggacaatgttctgtacaacaatttgacgagttttgcgggccacatgaatcgcttcggccaccttcgagggcgcatcggtcatcaacaccacgtccgctgtttcaatggcagcatcggagcccaatccccccatggctataccgacatcggctcgggcaatcacgggggcatcattaataccatctcctacaaatgccagttttgctttacctgaaggatctagcagttgctcaatttcatcgactttttcttctggcaataattcagcaacaaaggcatctaaaccaatttgctgggcaacggattgagctactatttcactatccccggtcagcatcacggtcttttcaactcccatgcgttttaggtctcgaatcgcttgaaccgcatcttccttaatttcgtcggcaatcagaatatagcccccatagcgcccatctacggcgagatgaaccactgtaccggctacgtcacaggtatcatgatcgattttttctcgatgcaaaaggcgatcattgcccgcaatgacgacttgattttgcacaacggctcgaatgccatgcccagcaatttcttcgtagtcggccacttcagagtcagcgatcgactgggcataagcttcgcggatcgatagggcaatgggatgagtggaatgggattcggcttttgcggctagggttaacaattctgattcagaaaatccatttttggtaacgacctgagtaaccttaaatgtgcctttggttaaggttccagttttatcaaacacaacggttttaacggccgtgagggagtctaaaaaggtagatcccttgatcaaaatcccatgtttggcggctcccccaatgccaccaaaatagccgagcggaatgctgatcaccaatccacagggacaggaaatcaccaacagaaccaaggcacgataaacccaatcggctcgatccgccccagggatgaacagaggtggtaacagggcaacggctaacgaaagaaagacgataacgggagtgtagtagcgggcaaattgggtgatgaatttctcggtggatgcctttttgctagacgcattttcaaccagatcgagcactttggcgatcgaagattcactaaatagtttggtgactcgaattgtcaacacacccgattggttgatcatgcctgcaagaatggtatctccaggcttaactgtgccgggcaccgattcgccagtcagtgctgaagtatctacctgtgaagttccccctaaaatctcgccatccaggggcactttttcacccggctttaccaaaattaagtcatcaacctgtaccgtttcgggggaaacttgttgcactgtaccattgcgcttcaagttcgcggtatcgggacgtgcttccagtaaagctttgatggaacggcgcgatcgcccaacggagtattcttgaaatagctctcccactcgaaagaacaacatcacggccacggcttcggggagttgatgaattgccaaggcccccaaggtggcgatcgtcattaaaaagttttcatcaaaaatctgccctctgagaatattgcgtcccgctgttttgagcactgtccaaccactcaagagataggcaggaataatcactgcaaattcagcaatctgacctggcgtgttgtgtaacggttgctcgaacaaaatagcaatggtaaagagggcgatcgccgtcaggacgggcagtagttcttgtttcagattaaattcaccagcaccatgactatggctatggccttcttccctatgggagtggggatgtttatgaccattaagcgttacagatgattttggctctgcaagggtgtaacccagggcggcgatccgttcttgaatggttatttcactgacctgcttgggatcataggtaacagttaatcgtcccgtggccaccgtgacagaggcctctgctactcccttcaatctttccaagctaccttcaatttttaacttgcaactagtgcagtccatgccgccgacttgcatttgctgagttttgagcggtgaagattgggtcatatgttaactcctgatgtgtggaaggaattgattacccttgaatggttctatcttaaaatttccccttccaggttagattcggttttcaggaaagaaggtggggggattgccgaaattacatttctagccgcaatttttagtaaaaaaaagatgagtttttacctcaccttaagtaaatatttgagtggcaaaacaaaatggtaaaaatagctaagcttccaccgccctatggatttttggaaggaagtcttaggttgtg

SEQ ID NO: 23 - DNA, 11528 bp, Artificial
  plasmid #1489 pAQ4::PhspA-ziaA-Pind(7002)-PDCdsrA-Prbc*-synADH(deg) standard
tcgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctcagcaagtttcatcccgaccccctcagggtcgggatttttttattgtactagtaacgcccggttgccaccgggcgttttttattccgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgctcgacaatcagcgacgaagtgctttccaaccactaaatcccgattacgggactgaaacactagaccctattctccaaaccttacggagcgtcccactttccaaccactaaatcccgattacgggactgaaacaggacgatagcgatggccccaatgcagaccccaatgactttccaaccactaaatcccgattacgggactgaaactatgaggtggcctgcgatttgctctactgcctcgatgctttccaaccactaaatcccgattacgggactgaaaccagatcgcagtcagcaaaacagccaaacttctcagggctttccaaccactaaatcccgattacgggactgaaacgttaaattctccaatccgagaacgctggcaacgccatcatctttccaaccactaaatcccgattacgggactgaaacaagtcccgataacagtcaactctataagcaaatctagctttccaaccactaaatcccgattacgggactgaaacgcaagtgatatgcaccacctccttcatcttactgctttccaaccactaaatcccgattacgggactgaaacatgagcccaacatgaaaccatttcactcccctgcctactttccaaccactaaatcccgattacgggactgaaacaaatcacgaattacccccttgtaagccttgtcgttctttccaaccactaaatcccgattacgggactgaaaccactcaaattgcaccctcatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatcactagtactgaaacgcctgcaattctccacaaaacaggctactttcccgctttccaaccactaaatcccgactacgggactgaaacatctctgtagtaatctctgtatatatagacgagtctttccaaccactaaatcccgactacgggactgaaacacgcaacatcgaggatcaccgccctgtcccccttgggactttccaaccactaaatcccgactacgggactgaaactggccatcgataaccgcattggcgacatcagccgccgcctttccaaccactaaatcccgactcgggactgaaaccaggtcaaaggctttctggtgggtatctcgcttgactttccaaccactaaatcccgactacgggactgaaactttttcttttgcgttgtgatttcttgaacataatcactttccaaccactaaatcccgactacgggactgaaacccgtcctggcgatggtttcggcgcgacgttccgctttccaaccactaaatcccgactacgggactgaaacatcacggggattgatgatgcggccgctctagacacggcccggtctcgacaacggggaggggcaggattaggccttgccattgcccaggcgatcgccgttaagcatcaaggtgttttgactgtagagagtgaattgggtcagggaagtttattcaccattcgcttatcaatatgtccgccaccataccaacagtcccgagagtgtaccaaaaatctcacctaacagggtatggatactgtcaatgcactttacttagcaatccgagtagcattcaaaatcgctaacagtgctactcccacatccgcaaacaccgcctcccagagggttgccaagccaatggtgcccagggcgataaaaagtgctttaatccctagggctaggacaatgttctgtacaacaatttgacgagttttgcgggccacatgaatcgcttcggccaccttcgagggcgcatcggtcatcaacaccacgtccgctgtttcaatggcagcatcggagcccaatccccccatggctataccgacatcggctcgggcaatcacgggggcatcattaataccatctcctacaaatgccagttttgctttacctgaaggatctagcagttgctcaatttcatcgactttttcttctggcaataattcagcaacaaaggcatctaaaccaatttgctgggcaacggattgagctactatttcactatccccggtcagcatcacggtcttttcaactcccatgcgttttaggtctcgaatcgcttgaaccgcatcttccttaatttcgtcggcaatcagaatatagcccccatagcgcccatctacggcgagatgaaccactgtaccggctacgtcacaggtatcatgatcgattttttctcgatgcaaaaggcgatcattgcccgcaatgacgacttgattttgcacaacggctcgaatgccatgcccagcaatttcttcgtagtcggccacttcagagtcagcgatcgactgggcataagcttcgcggatcgatagggcaatgggatgagtggaatgggattcggcttttgcggctagggttaacaattctgattcagaaaatccatttttggtaacgacctgagtaaccttaaatgtgcctttggttaaggttccagttttatcaaacacaacggttttaacggccgtgagggagtctaaaaaggtagatcccttgatcaaaatcccatgtttggcggctcccccaatgccaccaaaatagccgagcggaatgctgatcaccaatccacagggacaggaaatcaccaacagaaccaaggcacgataaacccaatcggctcgatccgccccagggatgaacagaggtggtaacagggcaacggctaacgaaagaaagacgataacgggagtgtagtagcgggcaaattgggtgatgaatttctcggtggatgcctttttgctagacgcattttcaaccagatcgagcactttggcgatcgaagattcactaaatagtttggtgactcgaattgtcaacacacccgattggttgatcatgcctgcaagaatggtatctccaggcttaactgtgccgggcaccgattcgccagtcagtgctgaagtatctacctgtgaagttccccctaaaatctcgccatccaggggcactttttcacccggctttaccaaaattaagtcatcaacctgtaccgtttcgggggaaacttgttgcactgtaccattgcgcttcaagttcgcggtatcgggacgtgcttccagtaaagctttgatggaacggcgcgatcgcccaacggagtattcttgaaatagctctcccactcgaaagaacaacatcacggccacggcttcggggagttgatgaattgccaaggcccccaaggtggcgatcgtcattaaaaagttttcatcaaaaatctgccctctgagaatattgcgtcccgctgttttgagcactgtccaaccactcaagagataggcaggaataatcactgcaaattcagcaatctgacctggcgtgttgtgtaacggttgctcgaacaaaatagcaatggtaaagagggcgatcgccgtcaggacgggcagtagttcttgtttcagattaaattcaccagcaccatgactatggctatggccttcttccctatgggagtggggatgtttatgaccattaagcgttacagatgattttggctctgcaagggtgtaacccagggcggcgatccgttcttgaatggttatttcactgacctgcttgggatcataggtaacagttaatcgtcccgtggccaccgtgacagaggcctctgctactcccttcaatctttccaagctaccttcaatttttaacttgcaactagtgcagtccatgccgccgacttgcatttgctgagttttgagcggtgaagattgggtcatatgttaactcctgatgtgtggaaggaattgattacccttgaatggttctatcttaaaatttccccttccaggttagattcggttttcaggaaagaaggtggggggattgccgaaattacatttctagccgcaatttttagtaaaaaaaagatgagtttttacctcaccttaagtaaatatttgagtggcaaaacaaaatggtaaaaatagctaagcttccaccgccctatggatttttggaaggaagtcttaggttgtg